(19) United States
(12) Patent Application Publication
(10) Pub. No.: US 2008/0025977 A1
(43) Pub. Date: Jan. 31, 2008

(54) Cytotoxicity mediation of cells evidencing surface expression of CD59

(75) Inventors: David S. F. Young, Toronto (CA); Helen P. Findlay, Toronto (CA); Susan E. Hahn, Toronto (CA); Lisa M. Cechetto, Seoul (KR); Luis A. G. de Cruz, Toronto (CA)

Correspondence Address: MCHALE & SLAVIN, P.A., 2855 PGA BLVD, PALM BEACH GARDENS, FL, 33410, US

(73) Assignee: Arius Research, Inc.

(21) Appl. No.: 11/807,681

(22) Filed: May 30, 2007

Related U.S. Application Data
(63) Continuation-in-part of application No. 11/361,153, filed on Feb. 24, 2006.
Application No. 11/361,153 is a continuation-in-part of application No. 10/944,664, filed on Sep. 15, 2004, now Pat. No. 7,195,764.
Application No. 10/944,664 is a continuation-in-part of application No. 10/413,755, filed on Apr. 14, 2003, now Pat. No. 6,794,494.

(51) Int. Cl. A61K 39/395; A61P 43/00; C07K 16/18; G01N 33/53

(52) U.S. Cl. 424/133.1; 424/138.1; 424/178.1; 435/7.21; 530/387.3; 530/387.7

(57) ABSTRACT

This invention relates to the staging, diagnosis and treatment of cancerous diseases (both primary tumors and tumor metastases), particularly to the mediation of cytotoxicity of tumor cells; and most particularly to the use of cancerous disease modifying antibodies (CDMAB), optionally in combination with one or more CDMAB/chemotherapeutic agents, as a means for initiating the cytotoxic response. The invention further relates to binding assays, which utilize the CDMAB of the instant invention. The anti-cancer antibodies can be conjugated to toxins, enzymes, radioactive compounds, cytokines, interferons, target or reporter moieties and hematogenous cells.

### REFERENCE TO RELATED APPLICATIONS

This application is a continuation-in-part to U.S. patent application Ser. No. 11/361,153 filed Feb. 24, 2006 which is a continuation-in-part to U.S. patent application Ser. No. 10/944,664 filed Sep. 15, 2004 which is a continuation-in-part to U.S. patent application Ser. No. 10/413,755, filed Apr. 14, 2003, now U.S. Pat. No. 6,794,494, and is a continuation-in-part to U.S. patent application Ser. No. 11/067,366, filed Feb. 25, 2005, which relies upon U.S. Provisional Application No. 60/548,667, filed Feb. 26, 2004, the contents of each of which are herein incorporated by reference.

### FIELD OF THE INVENTION

This invention relates to the diagnosis and treatment of cancerous diseases, particularly to the mediation of cytotoxicity of tumor cells; and most particularly to the use of cancerous disease modifying antibodies (CDMAB), optionally in combination with one or more CDMAB/chemotherapeutic agents, as a means for initiating the cytotoxic response. The invention further relates to binding assays, which utilize the CDMAB of the instant invention.

### BACKGROUND OF THE INVENTION

CD59 is an 18-20 kDa glycosyl phosphatidylinositol (GPI)-anchored membrane glycoprotein. It was initially isolated from the surface of human erythrocytes, and functions as an inhibitor of complement activation. Several antibodies that were developed to enhance complement-mediated lysis were subsequently found to target CD59. Their independent development led to the multitude of names by for CD59, including MEM-43 antigen, membrane inhibitor of reactive lysis (MIRL), H19, membrane attack complex-inhibitory factor (MACIF), homologous restriction factor with m.w. 20,000 (HRF20) and protectin (Walsh, Tone et al. 1992).

The CD59 antigen has been well characterized by amino acid analysis and NMR. It consists of 128 amino acids, of which the first 25 comprise a signal sequence. There are 10 cysteine residues, which result in a tightly folded molecule. The asparagine residue at position 18 is known to be N-glycosylated, while the asparagine residue at position 77 is linked to the GPI anchor. The C-terminus residues are characteristic of GPI-anchored proteins (Davies and Lachmann 1993).

CD59 was initially discovered on the surface of human erythrocytes, but is a widely expressed molecule. A large collection of data on cellular distribution from flow cytometry, immunohistochemistry and Northern blot analysis has revealed expression on many types of cells and tissues, including hematopoietic cells such as, platelets, leukocytes and fibroblasts, as well as erythrocytes (Meri, Waldmann et al. 1991). CD59 is abundant on vascular and ductal endothelium throughout the body, particularly in kidneys, bronchus, pancreas, skin epidermis and biliary and salivary glands (Meri, Waldmann et al. 1991). Expression has been noted in the lung, liver, placenta, thyroid and spermatozoa (Davies and Lachmann 1993). Soluble forms of CD59 have been detected in saliva, urine, tears, sweat, cerebrospinal fluid, breast milk, amniotic fluid and seminal plasma (Davies and Lachmann 1993). The origin of soluble CD59 has yet to be determined; whether it is secreted, cleaved by phospholipases or shed from cells by other means remains unknown (Davies and Lachmann 1993). CD59 appears to be absent from many B cell lines, CNS tissue, liver parenchyma and pancreatic Islets of Langerhans (Meri, Waldmann et al. 1991).

Although CD59 is widely expressed in normal cells and tissues, it is also widely expressed on malignant tumors. There is evidence that the expression of CD59 is increased in certain types of cancer compared to normal tissue and that the level of expression correlates with the stage of differentiation of the tumor. Moderate to high levels of CD59 expression have been reported in thyroid, prostate, breast, ovarian, lung, colorectal, pancreatic, gastric, renal and skin cancers as well as in malignant glioma, leukemia and lymphoma (Fishelson, Donin et al., 2003).

With the exception of tumor grade, no correlation is observed between CD59 expression and tumor/patient characteristics such as tumor type, size, vascular invasion, patient age, gender or menopausal status (breast cancer only) (Madjd, Pinder et al., 2003; Watson, Durrant et al., 2006). In studies using different tumor tissues that include breast, colorectal and prostate, CD59 expression correlates strongly with moderate to well-differentiated tumor grades (Madjd, Pinder et al., 2003; Watson, Durrant et al., 2006, Jarvis, Li et al., 1997; Koretz, Bruderlein et al., 1993). However, the association of CD59 expression on well-differentiated tumors with patient prognosis remains unresolved. Two separate studies using breast and colorectal cancer samples show that CD59 expression in highly differentiated cells correlates with good patient prognosis (Madjd, Pinder et al., 2003; Koretz, Bruderlein et al., 1993). Alternatively, in another study using colorectal cancer tissue, Watson et al. reported that the correlation between high CD59 levels and differentiated tumor grade can be sub-divided into early and late stage disease. These authors show that high CD59 levels found in well-differentiated early and late stage tumors is associated with a decrease in disease specific patient survival (Watson, Durrant et al., 2006).

Conversely, de-differentiated tumor cells correlates best with an absence of CD59 staining, which may have implications for metastasis. Several studies suggest that increased CD59 expression is inversely correlated with tumor metastasis. In breast carcinomas and colorectal cancers, high CD59 expression occurs in tumor samples without metastasis (Madjd, Pinder et al., 2003; Koretz, Bruderlein et al., 1993). Similarly, a low percentage of cells with high CD59 levels are found in colorectal metastatic tumors in the liver (Hosch, Scheunemann et al., 2001). Also, CD59 expression in squamous cell carcinomas of the head and neck are only elevated in samples with T1/T2N0M0 tumor grades and not in tumor grades beyond N1 and M1 (Ravindranath, Shuler et al., 2006).

The most characterized function of CD59 is its ability to inhibit the formation of the membrane attack complex (MAC) following complement activation. MAC formation is the final event in the complement cascade in which a pore is formed in the cellular membrane that ultimately leads to lysis of the cell. CD59 binds to C5b-8 and interferes with the subsequent polymerization of C9 molecules, the step that is required for MAC formation. Competition and mutational analysis of the epitopes of CD59, done with blocking and non-blocking monoclonal antibodies, has mapped the location of the active site of CD59 and has identified the amino acids Tyr-40, Arg-53 and Glu-56 to be necessary for CD59 activity (Bodian, Davies et al., 1997).

Complement activation results in either destruction of the targeted cell or cell activation, which recruits leukocytes, contracts surrounding smooth muscle and increases vascular permeability. Complement also plays a role in antibody-dependent cellular cytotoxicity (ADCC) and complement-dependent cellular cytotoxicity (CDCC). This can lead to an inflammatory response that could damage targeted tissues if poorly regulated. CD59 and other complement inhibitory proteins such as complement receptor type-1 (CR1; CD35), membrane cofactor protein (MCP; CD46) and decay accelerating factor (DAF; CD55) function to counter excessive activation of the complement cascade to prevent autologous tissue damage. It has been postulated that differential expression of complement inhibitory proteins such as CD59 may contribute to enhanced resistance to complement activation that malignant tumors often acquire (Jarvis, Li et al. 1997).

To evaluate whether resistance to complement by tumor cells can be overcome by targeting CD59, the ability of the CD59 blocking antibody YTH53.1 to enhance lysis of tumor cells has been evaluated in vitro. In a study using three-dimensional microtumor spheroids (MTS) with breast cancer (T47D cell line) and ovarian teratocarcinoma (PA-1 cell line) cells, the ability of this antibody to block CD59 activity and thus complement resistance has been measured. MTS are multicellular aggregates that grow in culture and represent a model closer to that observed in vivo than monolayer or suspension cultures. Previous work by this group has shown that PA-1 cells grown as MTS are more resistant to complement lysis than PA-1 cells grown in suspension. Cytotoxicity was measured by a chromium release assay and cell damage was visualized by uptake of propidium iodide (PI) following pre-treatment of MTS with biotinylated YTH53.1. Biotinylation of YTH53.1 retains its affinity for CD59 but eliminates its capacity to activate the classical complement pathway. Rabbit anti-human polyclonal antibody raised against breast cancer cells (S2 cell line) was used to activate the classical complement pathway. Overnight incubation with biotinylated YTH53.1 led to total infiltration of the MTS, and the chromium release assay showed killing of 33 percent of cells after a 1 to 2-hour lag phase in the presence of biotinylated YTH53.1, S2 and human complement. Under the same treatment, electron microscopy revealed the average T47D tumor volume decreased 28 percent. Fluorescence microscopy following PI incubation revealed several layers of cell death on T47D and PA-1 MTS. These results indicate that an anti-CD59 antibody that can block CD59 inhibitory activity can increase the complement-mediated lysis of tumor cells in vitro (Hakulinen and Meri 1998).

In another study, resistance to complement-mediated lysis by the human metastatic prostate adenocarcinoma cell lines DU145 and PC3 could be overcome in vitro by treating with YTH53.1. Chromium release assay was used to measure cell death in the presence and absence of YTH53.1 and biotinylated YTH53.1. In the absence of CD59 antibodies, both cell lines were completely resistant to complement-mediated lysis; however, treatment with YTH53.1 partially overcame this resistance by killing 56 percent of PC3 cells and 34 percent of DU145 cells. Treatment with biotinylated-YTH53.1 was less effective in overcoming complement resistance; 47 percent of PC3 and 20 percent of DU145 cells were killed. The higher expression of CD59 by PC3 compared with DU145 cells and possibly its greater dependence on CD59 expression and function in resisting complement mediated lysis is reflected by the increased sensitivity of PC3 compared to DU145. The differential effect of the native and biotinylated antibody demonstrates the enhanced effect of both activating the classical complement pathway and neutralization of CD59 (Jarvis, Li et al. 1997). However, the bulk of the activity of the antibody may be attributed to the blocking of complement inhibition (neutralization of CD59), as adding complement activation by the classical pathway only increases activity by a marginal amount (e.g. 47 percent for biotinylated-YTH53.1 versus 56 percent for YTH53.1 on PC3 cells) (Jarvis, Li et al. 1997). This study together with the one described previously demonstrates that targeting CD59 using an antibody may be an effective therapy for blocking resistance to complement activation in malignant tumors.

In an alternative approach, Harris et al. aimed to specifically target CD59 on tumor cells in vitro using engineered bi-specific antibodies. CD59 was neutralized using one of two different bispecific F(ab′gamma)2 antibody constructs which contained both cell-targeting (anti-CD19 or anti-CD38) and CD59-neutralizing moieties. In these experiments, Fab′gamma Fc gamma2 chimeric antibody (specific for human CD37) was used to activate the classical pathway of human complement on neoplastic B lymphoid cells (Raji). Neutralization of CD59 with either bi-specific constructs lysed 15-25 percent of Raji cells. In a mixture of target (Raji) and bystander (K562) cells, the anti-CD38×anti-CD59 bi-specific construct could be specifically delivered to Raji, avoiding significant uptake on CD59-expressing bystander cells. The anti-CD19×anti-CD59 bi-specific antibody bound equally well to either cell type indicating that the cell-specific targeting was dependent upon the high-affinity anti-tumor cell Fab′gamma (Harris, Kan et al., 1997). Although the premise of targeting tumor specific CD59 to avoid affecting normal bystander cells using bi-specific antibodies is appealing, these antibodies are limited by the affinity of the antibody to the tumor specific target. Furthermore, bi-specific antibodies may be complicated by the effect of targeting another tumor specific antigen that may result in pro-tumorgenic outcomes. Also, in the study described, the bi-specific antibodies are limited by the requirement for pre-activation of complement to enhance cell lysis. The use of a mono-specific antibody to CD59 with complement activating capability may be a less complicated and potentially more effect therapeutic tool. To date, there has been no in vivo analysis of the anti-CD59 antibody YTH53.1.

Tumor survival is also associated with CD59 expression during the acquisition of resistance to other forms of therapy. An inverse relationship between the clinical efficacy of Rituximab (Rituxan®, Genentech, San Francisco, Calif.) and CD59 levels has been described on lymphoma cells. The chimeric monoclonal antibody Rituximab is directed against the CD20 antigen and has been approved for use in treatment of non-Hodgkin's lymphoma (NHL). However, many patients that are CD20⁺ are unresponsive to treatment and most patients who do respond will eventually develop resistance to treatment. This is likely due to induction of complement inhibitors such as CD59. Using Rituximab-resistant B-lymphoma cell lines (RAMOS) with repeated exposure to a low concentration of Rituximab and complement, Takai et al. demonstrated that CD59 expression is increased during the establishment of resistant to Rituximab and complement (Takai et al., 2006). In response to the inhibition by antihormones, breast cancer cells recruit alternative signaling to limit maximal anti-tumor effects of oestrogen receptor (ER) blockade. A substantial increase in CD59 expression during response of MCF-7 cells to the antioestrogens tamoxifen or faslodex has been reported and shown to be transient during the acute phase of antioestrogen inhibition, with gene expression level subsequently declining once therapeutic resistance was acquired (Shaw, Gee et al., 2005). Targeting CD59 with antibodies is therefore also a potentially effective therapeutic approach to overcoming resistance to other cancer therapeutics in those cancers in which there is increased CD59 expression.

Use of anti-CD59 antibodies to increase CDCC as a means to overcome resistance to other therapies has been investigated. Rituxan-resistant NHL and MM cell lines express CD59 in the presence of complement in vitro, whereas Rituxan-sensitive NHL and MM cell lines do not express CD59. Pre-incubation of one of the resistant cell lines with an anti-CD59 antibody (YTH53.1) sensitized the cells to treatment with Rituximab and human complement. Also, high expression levels of CD59 have also been exhibited on tumors isolated from patients that are CD20⁺ but have had disease progression with Rituximab treatment (Treon, Emmanouilides et al. 2005).

In another study, a human mAb, directed against CD59 (MB-59) and isolated as single-chain variable fragments (scFv) from a human antibody library and engineered to contain the Hinge-CH2-CH3 domains of human IgG1, was used to evaluate the effect of targeting CD59 on two B lymphoma cell lines Karpas 422 and Hu-SCID1 that had undergone complement-mediated damage stimulated by Rituximab. In this assay, in which residual cells were measured by the MTT assay after antibody treatment, the number of cells sensitized by Rituximab and killed by complement was about 30 percent, but doubled when MB-59 was added to the test system (Ziller et al., 2005). Use of MB-59 alone was ineffective in enhancing complement mediated cytotoxicity. Therefore, treatment of Rituximab sensitized the tumor cells while the addition of anti-CD59 antibodies helped to overcome the partial resistance to Rituximab thereby making the tumor more responsive to immunotherapy or other treatments. Like YTH53-1, MB-59, to date, has not been analyzed for efficacy in vivo.

In addition to its role in complement regulation, CD59 has been implicated in angiogenesis as well. In a study by vanBeijnun et al., serial analysis of gene expression-(SAGE) tags were generated from tumor and normal endothelial cells (EC) and compared by suppression subtractive hybridization (SSH). From colon carcinoma tissues, non-malignant angiogenic placental tissues, and nonangiogenic normal tissues, CD59 was identified among four surface-expressing tumor angiogenesis genes (TAGs) to be overexpressed in tumor endothelium compared with angiogenic and nonangiogenic endothelium. Antibodies targeting CD59 inhibited angiogenesis as measured in EC tube formation (in vitro) and in the chick chorioallantoic membrane (CAM) (in vivo) assays (vanBeijnum, Ding et al., 2006). Treatment of cancer with anti-CD59 antibodies may have additional efficacy through the inhibition of angiogenesis in tumors.

In light of the differential expression of CD59 in various cancers, its induction during development of drug resistance and its role in angiogenesis, the abundance of CD59 on normal tissue is considered a barrier to using anti-CD59 antibodies as a targeted therapeutic. Paroxysmal nocturnal hemoglobinuria (PNH) is a rare heritable disorder that affects hematopoietic stem cells, resulting in cells that are abnormally sensitized to complement attack (Davies and Lachmann 1993). The symptoms include chronic hemolysis, anemia and thrombosis (Sugita and Masuho 1995). Cells affected by PNH, including erythrocytes, granulocytes, monocytes, platelets and sometimes lymphocytes, are deficient in GPI-anchored proteins such as acetylcholinesterase, LFA-3, HUPAR and complement regulator proteins CD35, CD46, CD55 and CD59 (Davies and Lachmann 1993). There is a single reported case of an individual that is completely lacking CD59 but none of the other complement regulatory GPI-anchored proteins. This deficiency is associated with PNH-like symptoms such as hemolytic anemia and thrombosis (Davies and Lachmann 1993). Although there are undesirable effects associated with lack of CD59 function, this individual proves that complete loss is non-lethal. Hemolytic side effects are a side effect of decreased CD59 expression and may be limiting in the use of CD59 antibodies clinically.

A mouse model in which one of the CD59 genes has been knocked out has demonstrated that CD59 deficiency is non-lethal in vivo. Mice express two forms of CD59, CD59a and CD59b. CD59a is widely expressed in various mouse tissues including blood cells, whereas CD59b expression has only been identified in the testis. Miwa et al. generated CD59a-deficient mice in order to assess the role of CD59 to protect erythrocytes from spontaneous complement attack in vivo. These knockout mice develop and live normally without any signs of hemolytic anemia and do not have elevated hemoglobin levels. Despite erythrocytes being more sensitive to induced complement attack by injection with cobra venom factor (CVF), erythrocyte elimination from spontaneous complement attack is not significantly elevated as compared to wild type (Miwa, Zhou et al. 2002).

Lastly, a F(ab′)₂ fragment of 6D1, a mouse monoclonal antibody directed against a 21-kDa membrane glycoprotein called rat inhibitory protein (RIP), the rat homologue of human CD59, has been administered to a group of male Wistar rats without significant side effects. In the same study, fragments of 512, an antibody directed against a different rat membrane-associated complement regulatory protein, was also administered. Following injection of 6D1 fragments, binding was detected in lung, heart and liver without any change in heart rate or blood pressure. The only observed effects were a small increase in leukocyte count and decrease in erythrocyte count; there was no change in the number of platelets. In contrast, injection with 512 fragments resulted in a rapid increase in blood pressure, a rapid decrease in leukocytes and platelets, and a continuously increasing erythrocyte count up to 2 hours following injection (Matsuo, Ichida et al. 1994). To date, there are no reports of any full-length, naked anti-CD59 antibodies exhibiting therapeutic efficacy in clinical studies or in preclinical cancer models in vivo.

Monoclonal Antibodies as Cancer Therapy: Each individual who presents with cancer is unique and has a cancer that is as different from other cancers as that person's identity. Despite this, current therapy treats all patients with the same type of cancer, at the same stage, in the same way. At least 30 percent of these patients will fail the first line therapy, thus leading to further rounds of treatment and the increased probability of treatment failure, metastases, and ultimately, death. A superior approach to treatment would be the customization of therapy for the particular individual. The only current therapy which lends itself to customization is surgery. Chemotherapy and radiation treatment cannot be tailored to the patient, and surgery by itself, in most cases is inadequate for producing cures.

With the advent of monoclonal antibodies, the possibility of developing methods for customized therapy became more realistic since each antibody can be directed to a single epitope. Furthermore, it is possible to produce a combination of antibodies that are directed to the constellation of epitopes that uniquely define a particular individual's tumor.

Having recognized that a significant difference between cancerous and normal cells is that cancerous cells contain antigens that are specific to transformed cells, the scientific community has long held that monoclonal antibodies can be designed to specifically target transformed cells by binding specifically to these cancer antigens; thus giving rise to the belief that monoclonal antibodies can serve as “Magic Bullets” to eliminate cancer cells. However, it is now widely recognized that no single monoclonal antibody can serve in all instances of cancer, and that monoclonal antibodies can be deployed, as a class, as targeted cancer treatments. Monoclonal antibodies isolated in accordance with the teachings of the instantly disclosed invention have been shown to modify the cancerous disease process in a manner which is beneficial to the patient, for example by reducing the tumor burden, and will variously be referred to herein as cancerous disease modifying antibodies (CDMAB) or “anti-cancer” antibodies.

At the present time, the cancer patient usually has few options of treatment. The regimented approach to cancer therapy has produced improvements in global survival and morbidity rates. However, to the particular individual, these improved statistics do not necessarily correlate with an improvement in their personal situation.

Thus, if a methodology was put forth which enabled the practitioner to treat each tumor independently of other patients in the same cohort, this would permit the unique approach of tailoring therapy to just that one person. Such a course of therapy would, ideally, increase the rate of cures, and produce better outcomes, thereby satisfying a long-felt need.

Historically, the use of polyclonal antibodies has been used with limited success in the treatment of human cancers. Lymphomas and leukemias have been treated with human plasma, but there were few prolonged remission or responses. Furthermore, there was a lack of reproducibility and there was no additional benefit compared to chemotherapy. Solid tumors such as breast cancers, melanomas and renal cell carcinomas have also been treated with human blood, chimpanzee serum, human plasma and horse serum with correspondingly unpredictable and ineffective results.

There have been many clinical trials of monoclonal antibodies for solid tumors. In the 1980s there were at least four clinical trials for human breast cancer which produced only one responder from at least 47 patients using antibodies against specific antigens or based on tissue selectivity. It was not until 1998 that there was a successful clinical trial using a humanized anti-Her2/neu antibody (Herceptin®) in combination with CISPLATIN. In this trial 37 patients were assessed for responses of which about a quarter had a partial response rate and an additional quarter had minor or stable disease progression. The median time to progression among the responders was 8.4 months with median response duration of 5.3 months.

Herceptin® was approved in 1998 for first line use in combination with Taxol®. Clinical study results showed an increase in the median time to disease progression for those who received antibody therapy plus Taxol® (6.9 months) in comparison to the group that received Taxol® alone (3.0 months). There was also a slight increase in median survival; 22 versus 18 months for the Herceptin® plus Taxol® treatment arm versus the Taxol® treatment alone arm. In addition, there was an increase in the number of both complete (8 versus 2 percent) and partial responders (34 versus 15 percent) in the antibody plus Taxol® combination group in comparison to Taxol® alone. However, treatment with Herceptin® and Taxol® led to a higher incidence of cardiotoxicity in comparison to Taxol® treatment alone (13 versus 1 percent respectively). Also, Herceptin® therapy was only effective for patients who over express (as determined through immunohistochemistry (IHC) analysis) the human epidermal growth factor receptor 2 (Her2/neu), a receptor, which currently has no known function or biologically important ligand; approximately 25 percent of patients who have metastatic breast cancer. Therefore, there is still a large unmet need for patients with breast cancer. Even those who can benefit from Herceptin® treatment would still require chemotherapy and consequently would still have to deal with, at least to some degree, the side effects of this kind of treatment.

The clinical trials investigating colorectal cancer involve antibodies against both glycoprotein and glycolipid targets. Antibodies such as 17-1A, which has some specificity for adenocarcinomas, has undergone Phase 2 clinical trials in over 60 patients with only 1 patient having a partial response. In other trials, use of 17-1A produced only 1 complete response and 2 minor responses among 52 patients in protocols using additional cyclophosphamide. To date, Phase III clinical trials of 17-1A have not demonstrated improved efficacy as adjuvant therapy for stage III colon cancer. The use of a humanized murine monoclonal antibody initially approved for imaging also did not produce tumor regression.

Only recently have there been any positive results from colorectal cancer clinical studies with the use of monoclonal antibodies. In 2004, ERBITUX® was approved for the second line treatment of patients with EGFR-expressing metastatic colorectal cancer who are refractory to irinotecan-based chemotherapy. Results from both a two-arm Phase II clinical study and a single arm study showed that ERBITUX® in combination with irinotecan had a response rate of 23 and 15 percent respectively with a median time to disease progression of 4.1 and 6.5 months respectively. Results from the same two-arm Phase II clinical study and another single arm study showed that treatment with ERBITUX® alone resulted in an 11 and 9 percent response rate respectively with a median time to disease progression of 1.5 and 4.2 months respectively.

Consequently in both Switzerland and the United States, ERBITUX® treatment in combination with irinotecan, and in the United States, ERBITUX® treatment alone, has been approved as a second line treatment of colon cancer patients who have failed first line irinotecan therapy. Therefore, like Herceptin®, treatment in Switzerland is only approved as a combination of monoclonal antibody and chemotherapy. In addition, treatment in both Switzerland and the US is only approved for patients as a second line therapy. Also, in 2004, AVASTIN® was approved for use in combination with intravenous 5-fluorouracil-based chemotherapy as a first line treatment of metastatic colorectal cancer. Phase III clinical study results demonstrated a prolongation in the median survival of patients treated with AVASTIN® plus 5-fluorouracil compared to patients treated with 5-fluourouracil alone (20 months versus 16 months respectively). However, again like Herceptin® and ERBITUX®, treatment is only approved as a combination of monoclonal antibody and chemotherapy.

There also continues to be poor results for lung, brain, ovarian, pancreatic, prostate, and stomach cancer. The most promising recent results for non-small cell lung cancer came from a Phase II clinical trial where treatment involved a monoclonal antibody (SGN-15; dox-BR96, anti-Sialyl-LeX) conjugated to the cell-killing drug doxorubicin in combination with the chemotherapeutic agent TAXOTERE®. TAXOTERE® is the only FDA approved chemotherapy for the second line treatment of lung cancer. Initial data indicate an improved overall survival compared to TAXOTERE® alone. Out of the 62 patients who were recruited for the study, two-thirds received SGN-15 in combination with TAXOTERE® while the remaining one-third received TAXOTERE® alone. For the patients receiving SGN-15 in combination with TAXOTERE®, median overall survival was 7.3 months in comparison to 5.9 months for patients receiving TAXOTERE® alone. Overall survival at 1 year and 18 months was 29 and 18 percent respectively for patients receiving SNG-15 plus TAXOTERE® compared to 24 and 8 percent respectively for patients receiving TAXOTERE® alone. Further clinical trials are planned.

Preclinically, there has been some limited success in the use of monoclonal antibodies for melanoma. Very few of these antibodies have reached clinical trials and to date none have been approved or demonstrated favorable results in Phase III clinical trials.

The discovery of new drugs to treat disease is hindered by the lack of identification of relevant targets among the products of 30,000 known genes that could contribute to disease pathogenesis. In oncology research, potential drug targets are often selected simply due to the fact that they are over-expressed in tumor cells. Targets thus identified are then screened for interaction with a multitude of compounds. In the case of potential antibody therapies, these candidate compounds are usually derived from traditional methods of monoclonal antibody generation according to the fundamental principles laid down by Kohler and Milstein (1975, Nature, 256, 495-497, Kohler and Milstein). Spleen cells are collected from mice immunized with antigen (e.g. whole cells, cell fractions, purified antigen) and fused with immortalized hybridoma partners. The resulting hybridomas are screened and selected for secretion of antibodies which bind most avidly to the target. Many therapeutic and diagnostic antibodies directed against cancer cells, including Herceptin® and RITUXIMAB, have been produced using these methods and selected on the basis of their affinity. The flaws in this strategy are two-fold. Firstly, the choice of appropriate targets for therapeutic or diagnostic antibody binding is limited by the paucity of knowledge surrounding tissue specific carcinogenic processes and the resulting simplistic methods, such as selection by overexpression, by which these targets are identified. Secondly, the assumption that the drug molecule that binds to the receptor with the greatest affinity usually has the highest probability for initiating or inhibiting a signal may not always be the case.

Despite some progress with the treatment of breast and colon cancer, the identification and development of efficacious antibody therapies, either as single agents or co-treatments, have been inadequate for all types of cancer.

Prior Patents:

World application No. PCT/EP2006/009496 discloses the localization of CD59 as determined with a commercial antibody on colorectal carcinoma tissue. The antibody was then tested in an in vitro collagen-gel-based sprout-formation assay where no significant activity was detected. The antibody was then tested experimentally in the developing chorioallentoic membrane (CAM) of a chick embryo where it demonstrated inhibition of angiogenesis by 27 percent.

U.S. Pat. No. 5,750,102 discloses a process wherein cells from a patient's tumor are transfected with MHC genes which may be cloned from cells or tissue from the patient. These transfected cells are then used to vaccinate the patient.

U.S. Pat. No. 4,861,581 discloses a process comprising the steps of obtaining monoclonal antibodies that are specific to an internal cellular component of neoplastic and normal cells of the mammal but not to external components, labeling the monoclonal antibody, contacting the labeled antibody with tissue of a mammal that has received therapy to kill neoplastic cells, and determining the effectiveness of therapy by measuring the binding of the labeled antibody to the internal cellular component of the degenerating neoplastic cells. In preparing antibodies directed to human intracellular antigens, the patentee recognizes that malignant cells represent a convenient source of such antigens.

U.S. Pat. No. 5,171,665 provides a novel antibody and method for its production. Specifically, the patent teaches formation of a monoclonal antibody which has the property of binding strongly to a protein antigen associated with human tumors, e.g. those of the colon and lung, while binding to normal cells to a much lesser degree.

U.S. Pat. No. 5,484,596 provides a method of cancer therapy comprising surgically removing tumor tissue from a human cancer patient, treating the tumor tissue to obtain tumor cells, irradiating the tumor cells to be viable but non-tumorigenic, and using these cells to prepare a vaccine for the patient capable of inhibiting recurrence of the primary tumor while simultaneously inhibiting metastases. The patent teaches the development of monoclonal antibodies which are reactive with surface antigens of tumor cells. As set forth at col. 4, lines 45 et seq., the patentees utilize autochthonous tumor cells in the development of monoclonal antibodies expressing active specific immunotherapy in human neoplasia.

U.S. Pat. No. 5,693,763 teaches a glycoprotein antigen characteristic of human carcinomas and not dependent upon the epithelial tissue of origin.

U.S. Pat. No. 5,783,186 is drawn to Anti-Her2 antibodies which induce apoptosis in Her2 expressing cells, hybridoma cell lines producing the antibodies, methods of treating cancer using the antibodies and pharmaceutical compositions including said antibodies.

U.S. Pat. No. 5,849,876 describes new hybridoma cell lines for the production of monoclonal antibodies to mucin antigens purified from tumor and non-tumor tissue sources.

U.S. Pat. No. 5,869,268 is drawn to a method for generating a human lymphocyte producing an antibody specific to a desired antigen, a method for producing a monoclonal antibody, as well as monoclonal antibodies produced by the method. The patent is particularly drawn to the production of an anti-HD human monoclonal antibody useful for the diagnosis and treatment of cancers.

U.S. Pat. No. 5,869,045 relates to antibodies, antibody fragments, antibody conjugates and single chain immunotoxins reactive with human carcinoma cells. The mechanism by which these antibodies function is two-fold, in that the molecules are reactive with cell membrane antigens present on the surface of human carcinomas, and further in that the antibodies have the ability to internalize within the carcinoma cells, subsequent to binding, making them especially useful for forming antibody-drug and antibody-toxin conjugates. In their unmodified form the antibodies also manifest cytotoxic properties at specific concentrations.

U.S. Pat. No. 5,780,033 discloses the use of autoantibodies for tumor therapy and prophylaxis. However, this antibody is an antinuclear autoantibody from an aged mammal. In this case, the autoantibody is said to be one type of natural antibody found in the immune system. Because the autoantibody comes from “an aged mammal”, there is no requirement that the autoantibody actually comes from the patient being treated. In addition the patent discloses natural and monoclonal antinuclear autoantibody from an aged mammal, and a hybridoma cell line producing a monoclonal antinuclear autoantibody.

U.S. Patent Application 20050032128A1 discloses the use of anti-glycated CD59 antibodies for the treatment of diabetes.

### SUMMARY OF THE INVENTION

This application utilizes methodology for producing anti-cancer antibodies taught in the U.S. Pat. No. 6,180,357 patent for isolating hybridoma cell lines which encode for cancerous disease modifying monoclonal antibodies. These antibodies can be made specifically for one tumor and thus make possible the customization of cancer therapy. Within the context of this application, anti-cancer antibodies having either cell-killing (cytotoxic) or cell-growth inhibiting (cytostatic) properties will hereafter be referred to as cytotoxic. These antibodies can be used in aid of staging and diagnosis of a cancer, and can be used to treat tumor metastases. These antibodies can also be used for the prevention of cancer by way of prophylactic treatment. Unlike antibodies generated according to traditional drug discovery paradigms, antibodies generated in this way may target molecules and pathways not previously shown to be integral to the growth and/or survival of malignant tissue. Furthermore, the binding affinities of these antibodies are suited to requirements for initiation of the cytotoxic events that may not be amenable to stronger affinity interactions. Also, it is within the purview of this invention to conjugate standard chemotherapeutic modalities, e.g. radionuclides, with the CDMAB of the instant invention, thereby focusing the use of said chemotherapeutics. The CDMAB can also be conjugated to toxins, cytotoxic moieties, enzymes e.g. biotin conjugated enzymes, cytokines, interferons, target or reporter moieties or hematogenous cells, thereby forming an antibody conjugate. The CDMAB can be used alone or in combination with one or more CDMAB/chemotherapeutic agents.

The prospect of individualized anti-cancer treatment will bring about a change in the way a patient is managed. A likely clinical scenario is that a tumor sample is obtained at the time of presentation, and banked. From this sample, the tumor can be typed from a panel of pre-existing cancerous disease modifying antibodies. The patient will be conventionally staged but the available antibodies can be of use in further staging the patient. The patient can be treated immediately with the existing antibodies, and a panel of antibodies specific to the tumor can be produced either using the methods outlined herein or through the use of phage display libraries in conjunction with the screening methods herein disclosed. All the antibodies generated will be added to the library of anti-cancer antibodies since there is a possibility that other tumors can bear some of the same epitopes as the one that is being treated. The antibodies produced according to this method may be useful to treat cancerous disease in any number of patients who have cancers that bind to these antibodies.

In addition to anti-cancer antibodies, the patient can elect to receive the currently recommended therapies as part of a multi-modal regimen of treatment. The fact that the antibodies isolated via the present methodology are relatively non-toxic to non-cancerous cells allows for combinations of antibodies at high doses to be used, either alone, or in conjunction with conventional therapy. The high therapeutic index will also permit re-treatment on a short time scale that should decrease the likelihood of emergence of treatment resistant cells.

If the patient is refractory to the initial course of therapy or metastases develop, the process of generating specific antibodies to the tumor can be repeated for re-treatment. Furthermore, the anti-cancer antibodies can be conjugated to red blood cells obtained from that patient and re-infused for treatment of metastases. There have been few effective treatments for metastatic cancer and metastases usually portend a poor outcome resulting in death. However, metastatic cancers are usually well vascularized and the delivery of anti-cancer antibodies by red blood cells can have the effect of concentrating the antibodies at the site of the tumor. Even prior to metastases, most cancer cells are dependent on the host's blood supply for their survival and an anti-cancer antibody conjugated to red blood cells can be effective against in situ tumors as well. Alternatively, the antibodies may be conjugated to other hematogenous cells, e.g. lymphocytes, macrophages, monocytes, natural killer cells, etc.

There are five classes of antibodies and each is associated with a function that is conferred by its heavy chain. It is generally thought that cancer cell killing by naked antibodies are mediated either through antibody dependent cellular cytotoxicity (ADCC) or complement dependent cytotoxicity (CDC). For example murine IgM and IgG2a antibodies can activate human complement by binding the C-1 component of the complement system thereby activating the classical pathway of complement activation which can lead to tumor lysis. For human antibodies the most effective complement activating antibodies are generally IgM and IgG1. Murine antibodies of the IgG2a and IgG3 isotype are effective at recruiting cytotoxic cells that have Fc receptors which will lead to cell killing by monocytes, macrophages, granulocytes and certain lymphocytes. Human antibodies of both the IgG1 and IgG3 isotype mediate ADCC.

The cytotoxicity mediated through the Fc region requires the presence of effector cells, their corresponding receptors, or proteins e.g. NK cells, T-cells and complement. In the absence of these effector mechanisms, the Fc portion of an antibody is inert. The Fc portion of an antibody may confer properties that affect the pharmacokinetics of an antibody in vivo, but in vitro this is not operative.

Another possible mechanism of antibody mediated cancer killing may be through the use of antibodies that function to catalyze the hydrolysis of various chemical bonds in the cell membrane and its associated glycoproteins or glycolipids, so-called catalytic antibodies.

There are three additional mechanisms of antibody-mediated cancer cell killing. The first is the use of antibodies as a vaccine to induce the body to produce an immune response against the putative antigen that resides on the cancer cell. The second is the use of antibodies to target growth receptors and interfere with their function or to down regulate that receptor so that its function is effectively lost. The third is the effect of such antibodies on direct ligation of cell surface moieties that may lead to direct cell death, such as ligation of death receptors such as TRAIL R1 or TRAIL R2, or integrin molecules such as alpha V beta 3 and the like.

The clinical utility of a cancer drug is based on the benefit of the drug under an acceptable risk profile to the patient. In cancer therapy survival has generally been the most sought after benefit, however there are a number of other well-recognized benefits in addition to prolonging life. These other benefits, where treatment does not adversely affect survival, include symptom palliation, protection against adverse events, prolongation in time to recurrence or disease-free survival, and prolongation in time to progression. These criteria are generally accepted and regulatory bodies such as the U.S. Food and Drug Administration (F.D.A.) approve drugs that produce these benefits (Hirschfeld et al. Critical Reviews in Oncology/Hematolgy 42:137-143 2002). In addition to these criteria it is well recognized that there are other endpoints that may presage these types of benefits. In part, the accelerated approval process granted by the U.S. F.D.A. acknowledges that there are surrogates that will likely predict patient benefit. As of year-end 2003, there have been sixteen drugs approved under this process, and of these, four have gone on to full approval, i.e., follow-up studies have demonstrated direct patient benefit as predicted by surrogate endpoints. One important endpoint for determining drug effects in solid tumors is the assessment of tumor burden by measuring response to treatment (Therasse et al. Journal of the National Cancer Institute 92(3):205-216 2000). The clinical criteria (RECIST criteria) for such evaluation have been promulgated by Response Evaluation Criteria in Solid Tumors Working Group, a group of international experts in cancer. Drugs with a demonstrated effect on tumor burden, as shown by objective responses according to RECIST criteria, in comparison to the appropriate control group tend to, ultimately, produce direct patient benefit. In the pre-clinical setting tumor burden is generally more straightforward to assess and document. In that pre-McHale clinical studies can be translated to the clinical setting, drugs that produce prolonged survival in pre-clinical models have the greatest anticipated clinical utility. Analogous to producing positive responses to clinical treatment, drugs that reduce tumor burden in the pre-clinical setting may also have significant direct impact on the disease. Although prolongation of survival is the most sought after clinical outcome from cancer drug treatment, there are other benefits that have clinical utility and it is clear that tumor burden reduction, which may correlate to a delay in disease progression, extended survival or both, can also lead to direct benefits and have clinical impact (Eckhardt et al. Developmental Therapeutics Successes and Failures of Clinical Trial Designs of Targeted Compounds; ASCO Educational Book, 39^{th} Annual Meeting, 2003, pages 209-219). Using substantially the process of U.S. Pat. No. 6,180,357, and as disclosed in U.S. patents Ser. No. 11/361,153 and Ser. No. 11/067,366, the contents of each of which are herein incorporated by reference, the mouse monoclonal antibody, AR36A36.11.1 was obtained following immunization of mice with cells from human prostate tumor tissue. The AR36A36.11.1 antigen was expressed on the cell surface of a wide range of human cell lines from different tissue origins. The prostate cancer cell line LnCap was susceptible to the cytotoxic effects of AR36A36.11.1 in vitro.

The result of AR36A36.11.1 cytotoxicity against prostate cancer cells in vitro was further extended by demonstrating its anti-tumor activity in vivo (as disclosed in Ser. No. 11/067,366). AR36A36.11.1 prevented tumor growth and reduced tumor burden in a preventative in vivo model of human prostate cancer. On day 41 post-implantation, 5 days after the last treatment dose, the mean tumor volume in the AR36A36.11.1 treated group was 14 percent of the tumor volume in the buffer control-treated group (p=0.0009, t-test). In a PC-3 prostate cancer xenograft model, body weight can be used as a surrogate indicator of disease progression (Wang et al. Int J Cancer, 2003). By the end of the study (day 41), control animals exhibited a 27 percent decrease in body weight from the onset of the study. By contrast, the group treated with AR36A36.11.1 had a significantly higher body weight than the control group (p=0.017). Overall, the AR36A36.11.1-treated group lost only 6 percent of its body weight, much less than the 27 percent lost by the buffer control group. Therefore AR36A36.11.1 was well-tolerated and decreased the tumor burden and cachexia in a human prostate cancer xenograft model.

In addition to its anti-prostate cancer effects, AR36A36.11.1 demonstrated anti-tumor activity against SW1116 colon cancer cells in a preventative in vivo tumor model (as disclosed in Ser. No. 11/067,366). On day 55 post-implantation, 5 days after the last treatment dose, the mean tumor volume in the AR36A36.11.1-treated group was 51 percent of the tumor volume in the buffer control-treated group (p=0.0055, t-test). There were no clinical signs of toxicity throughout the study. Body weight measured at weekly intervals was a surrogate for well-being and failure to thrive. There was no significant difference in body weight between the groups at the end of the treatment period (p=0.4409, t-test). Therefore AR36A36.11.1 was well-tolerated and decreased the tumor burden in a human colon cancer xenograft model.

In addition, AR36A36.11.1 demonstrated anti-tumor activity against MDA-MB-231 breast cancer in a preventative in vivo tumor model (as disclosed in Ser. No. 11/067,366). AR36A36.11.1 completely prevented tumor growth and reduced tumor burden. On day 56 post-implantation, 6 days after the last treatment dose, the mean tumor volume in the AR36A36.11.1 treated group was 0 percent of the tumor volume in the isotype control-treated group (p=0.0002, t-test). There were no clinical signs of toxicity throughout the study. Body weight measured at weekly intervals was a surrogate for well-being and failure to thrive. There was no significant difference in body weight between the groups at the end of the treatment period (p=0.0676, t-test). Therefore AR36A36.11.1 was well-tolerated and decreased the tumor burden in a human breast cancer xenograft model.

Also, AR36A36.11.1 demonstrated anti-tumor activity against MDA-MB-231 breast cancer in an established in vivo tumor model (as disclosed in Ser. No. 11/067,366). AR36A36.11.1 prevented tumor growth and reduced tumor burden in this established in vivo model of human breast cancer. On day 83 post-implantation, 2 days after the last treatment dose, the mean tumor volume in the AR36A36.11.1-treated group was 46 percent of the tumor volume in the buffer control-treated group (p=0.0038, t-test). This corresponds to a mean T/C of 32 percent. There were no clinical signs of toxicity throughout the study. Body weight measured at weekly intervals was a surrogate for well-being and failure to thrive. There was no significant difference in body weight between the groups at the end of the treatment period (p=0.6493, t-test).

Treatment benefits were observed in several well-recognized models of human cancer disease suggesting pharmacologic and pharmaceutical benefits of this antibody for therapy in other mammals, including man. In toto, this data demonstrates that the AR36A36.11.1 antigen is a cancer associated antigen and is expressed on human cancer cells, and is a pathologically relevant cancer target.

As disclosed previously (Ser. No. 11/361,153), biochemical data indicated that the antigen recognized by AR36A36.11.1 is CD59. This was supported by studies that showed a monoclonal antibody (clone MEM-43, Serotec, Raleigh, N.C.) reactive against CD59 identifies proteins that were bound to AR36A36.11.1 by immunoprecipitation. The AR36A36.11.1 epitope does not appear to be carbohydrate dependent.

In order to validate the AR36A36.11.1 epitope as a drug target, the expression of AR36A36.11.1 antigen in normal human tissue sections was previously determined (as disclosed in Ser. No. 11/361,153). Binding of antibodies to 59 normal human tissues was performed using a human, normal organ tissue array (Imgenex, San Diego, Calif.). The AR36A36.11.1 antibody bound predominantly to epithelial tissues (endothelium of blood vessels of various organs, squamous epithelium of skin and tonsils, ductular epithelium of breast, nasal mucosal epithelium, acinar and ductal epithelium of salivary glands, bile duct epithelium of liver, acinar epithelium and Islet of Langerhans of pancreas, mucosal epithelium of urinary bladder and glandular epithelium of prostate). The AR36A36.11.1 antibody has demonstrated binding to human tissue that is consistent with that previously reported for anti-CD59 antibodies.

To further extend the potential therapeutic benefit of AR36A36.11.1, the frequency and localization of the antigen within various human cancer tissues was also determined (previously disclosed in Ser. No. 11/361,153). The AR36A36.11.1 antibody bound to 17/54 (32 percent) of tested tumors. The antibody bound strongly to 2/17 tumors, moderately to 2/17, weakly to 4/17 and equivocally to 9/17. The tissue specificity was for tumor cells and stromal blood vessels. Cellular localization was membranous cytoplasmic with diffuse staining pattern. Therefore, it has been demonstrated that the AR36A36.11.1 antigen is located on the membranes of a variety of tumor types. These results indicate that the AR36A36.11.1 antibody has potential as a therapeutic drug in a wide variety of cancers including but not limited to cancers of the skin, liver and pancreas.

The present invention describes the development and use of AR36A36.11.1, chimeric AR36A36.11.1 ((ch)AR36A36.11.1) and humanized variants (hu)AR36A36.11.1. AR36A36.11.1 was identified by its effect in a cytotoxic assay and in non-established and established tumor growth in animal models. This invention represents an advance in the field of cancer treatment in that it describes, for the first time, reagents that bind specifically to an epitope or epitopes present on the target molecule, CD59, and that also have in vitro cytotoxic properties, as a naked antibody, against malignant tumor cells but not normal cells, and which also directly mediate, as a naked antibody, inhibition of tumor growth and extension of survival in in vivo models of human cancer. This is an advance in relation to any other previously described anti-CD59 antibody, since none have been shown to have similar properties. It also provides an advance in the field since it clearly demonstrates, and for the first time, the direct involvement of CD59 in events associated with growth and development of certain types of tumors. It also represents an advance in cancer therapy since it has the potential to display similar anti-cancer properties in human patients. A further advance is that inclusion of these antibodies in a library of anti-cancer antibodies will enhance the possibility of targeting tumors expressing different antigen markers by determination of the appropriate combination of different anti-cancer antibodies, to find the most effective in targeting and inhibiting growth and development of the tumors.

In all, this invention teaches the use of the AR36A36.11.1 antigen as a target for a therapeutic agent, that when administered can reduce the tumor burden of a cancer expressing the antigen in a mammal, and can also lead to a prolonged survival of the treated mammal. This invention also teaches the use of CDMAB (AR36A36.11.1, (ch)AR36A36.11.1 and humanized variants, (hu)AR36A36.11.1), and its derivatives, and antigen binding fragments thereof, and cellular cytotoxicity inducing ligands thereof to target their antigen to reduce the tumor burden of a cancer expressing the antigen in a mammal, and lead to prolonged survival of the treated mammal. Furthermore, this invention also teaches the use of detecting the AR36A36.11.1 antigen in cancerous cells that can be useful for the diagnosis, prediction of therapy, and prognosis of mammals bearing tumors that express this antigen.

Accordingly, it is an objective of the invention to utilize a method for producing cancerous disease modifying antibodies (CDMAB) raised against cancerous cells derived from a particular individual, or one or more particular cancer cell lines, which CDMAB are cytotoxic with respect to cancer cells while simultaneously being relatively non-toxic to non-cancerous cells, in order to isolate hybridoma cell lines and the corresponding isolated monoclonal antibodies and antigen binding fragments thereof for which said hybridoma cell lines are encoded.

It is an additional objective of the invention to teach cancerous disease modifying antibodies, ligands and antigen binding fragments thereof.

It is a further objective of the instant invention to produce cancerous disease modifying antibodies whose cytotoxicity is mediated through antibody dependent cellular toxicity.

It is yet an additional objective of the instant invention to produce cancerous disease modifying antibodies whose cytotoxicity is mediated through complement dependent cellular toxicity.

It is still a further objective of the instant invention to produce cancerous disease modifying antibodies whose cytotoxicity is a function of their ability to catalyze hydrolysis of cellular chemical bonds.

A still further objective of the instant invention is to produce cancerous disease modifying antibodies which are useful for in a binding assay for diagnosis, prognosis, and monitoring of cancer.

Other objects and advantages of this invention will become apparent from the following description wherein are set forth, by way of illustration and example, certain embodiments of this invention.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 demonstrates the effect of AR36A36.11.1 on tumor growth in an established human PC-3 prostate cancer model. The vertical dashed lines indicate the period during which the antibody was intraperitoneally administered. Data points represent the mean+/−SEM.

FIG. 2 demonstrates the effect of AR36A36.11.1 on mouse body weight in an established PC-3 prostate cancer model. Data points represent the mean+/−SEM.

FIG. 3 demonstrates the effect of AR36A36.11.1 on tumor growth in an established human breast MDA-MB-468 cancer model. The vertical dashed lines indicate the period during which the antibody was intraperitoneally administered. Data points represent the mean+/−SEM.

FIG. 4 demonstrates the effect of AR36A36.11.1 on mouse body weight in an established MDA-MB-468 breast cancer model. Data points represent the mean SEM.

FIG. 5 demonstrates the effect of AR36A36.11.1 in a dose-response manner on tumor growth in an established human breast (MDA-MB-231) cancer model. The vertical dashed lines indicate the period during which the antibody was intraperitoneally administered. Data points represent the mean+/−SEM.

FIG. 6 demonstrates the effect of AR36A36.11.1 on mouse body weight in an established MDA-MB-231 breast cancer model. Data points represent the mean+/−SEM.

FIG. 7 demonstrates the effect of AR36A36.11.1 on tumor growth in a prophylactic NCI-H520 human lung squamous cell carcinoma model. The vertical dashed lines indicate the period during which the antibody was intraperitoneally administered. Data points represent the mean+/−SEM.

FIG. 8 demonstrates effect of AR36A36.11.1 on mouse survival in a prophylactic NCI-H520 human lung squamous cell carcinoma model. Data points represent the survival percentage.

FIG. 9 demonstrates the effect of AR36A36.11.1 on mouse body weight in a prophylactic NCI-H520 human lung squamous cell carcinoma model. Data points represent the mean+/−SEM.

FIG. 10. Western blot of a total membrane preparation of MDA-MB-231 breast cancer cells probed with different primary antibody solutions. Lanes 3 to 7 were probed with biotinylated AR36A36.11.1 mixed with 0.5 micrograms/mL, 5 micrograms/mL, 50 micrograms/mL, 500 micrograms/mL and 1000 micrograms/mL of non-biotinylated AR36A36.11.1 respectively. Lanes 9-13 were probed with biotinylated AR36A36.11.1 mixed with 0.5 micrograms/mL, 5 micrograms/mL, 50 micrograms/mL, 500 micrograms/mL and 1000 micrograms/mL of non-biotinylated 10A304.7 respectively. Lanes 15-19 were probed with biotinylated AR36A36.11.1 mixed with 0.5 micrograms/mL, 5 micrograms/mL, 50 micrograms/mL, 500 micrograms/mL and 1000 micrograms/mL of non-biotinylated 8B1B.1 respectively. Lanes 8 and 14 were incubated with negative control solution and lane 8 was not incubated in secondary solution. Lanes 1, 2 and 20 were incubated with TBST only.

FIG. 11. Western blot of a total membrane preparation of MDA-MB-231 breast cancer cells probed with different primary antibody solutions. Lanes 3 to 7 were probed with biotinylated 10A304.7 mixed with 0.5 micrograms/mL, 5 micrograms/mL, 50 micrograms/mL, 500 micrograms/mL and 1000 micrograms/mL of non-biotinylated 10A304.7 respectively. Lanes 9 to 13 were probed with biotinylated 10A304.7 mixed with 0.5 micrograms/mL, 5 micrograms/mL, 50 micrograms/mL, 500 micrograms/mL and 1000 micrograms/mL of non-biotinylated AR36A36.11.1 respectively. Lanes 15 to 19 were probed with biotinylated 10A304.7 mixed with 0.5 micrograms/mL, 5 micrograms/mL, 50 micrograms/mL, 500 micrograms/mL and 1000 micrograms/mL of non-biotinylated 8A3B.6 respectively. Lanes 8 and 14 were incubated with negative control solution and lane 8 was not incubated in secondary solution. Lanes 1, 2 and 20 were incubated with TBST only.

FIG. 12. Binding of 10A304.7 to CLIPS peptides synthesized based on CD59 amino acid sequence.

FIG. 13. Binding of AR36A36.11.1 to CLIPS peptides synthesized based on CD59 amino acid sequence.

FIG. 14. Amino acid sequence of CD59. The discontinuous epitope recognized by both 10A304.7 and AR36A36.11.1 is contained within the underlined sequences.

FIG. 15. Primers used in the PCR amplification of light chain.

FIG. 16. Primers used in the PCR amplification of heavy chain.

FIG. 17. Mouse AR36A36.11.1 VH Sequence. CDRs are underlined.

FIG. 18. Mouse AR36A36.11.1 VL Sequence. CDRs are underlined.

FIG. 19. Oligonucleotides used for the generation of chimeric and variant humanized AR36A36.11.1 VH sequences.

FIG. 20. Oligonucleotides used for the generation of chimeric and variant humanized AR36A36.11.1 VL sequences.

FIG. 21. Light chain and heavy chain expression vectors.

FIG. 22A and FIG. 22B. Humanized AR36A36.11.1 VH variants. CDRs are underlined.

FIG. 23A and FIG. 23B. Humanized AR36A36.11.1 VL variants. CDRs are underlined.

FIG. 24. Activities of humanized AR36A36.11.1 VH and VL variants.

FIG. 25 demonstrates the binding of humanized variants, chimeric and murine AR36A36.11.1 to the human breast cancer cell line MDA-MB-231.

FIG. 26 demonstrates the in vitro CDC activity of murine and humanized variants of AR36A36.11.1 on the human breast cancer cell line MDA-MB-231.

### DETAILED DESCRIPTION OF THE INVENTION

In general, the following words or phrases have the indicated definition when used in the summary, description, examples, and claims.

The term “antibody” is used in the broadest sense and specifically covers, for example, single monoclonal antibodies (including agonist, antagonist, and neutralizing antibodies, de-immunized, murine, chimeric or humanized antibodies), antibody compositions with polyepitopic specificity, single-chain antibodies, diabodies, triabodies, immunoconjugates and antibody fragments (see below).

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma (murine or human) method first described by Kohler et al., Nature, 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example.

“Antibody fragments” comprise a portion of an intact antibody, preferably comprising the antigen-binding or variable region thereof. Examples of antibody fragments include less than full length antibodies, Fab, Fab′, F(ab′)₂, and Fv fragments; diabodies; linear antibodies; single-chain antibody molecules; single-chain antibodies, single domain antibody molecules, fusion proteins, recombinant proteins and multispecific antibodies formed from antibody fragment(s).

An “intact” antibody is one which comprises an antigen-binding variable region as well as a light chain constant domain (C_{L}) and heavy chain constant domains, C_{H}1, C_{H}2 and C_{H}3. The constant domains may be native sequence constant domains (e.g. human native sequence constant domains) or amino acid sequence variant thereof. Preferably, the intact antibody has one or more effector functions.

Depending on the amino acid sequence of the constant domain of their heavy chains, intact antibodies can be assigned to different “classes”. There are five-major classes of intact antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called α, δ, ε, γ, and μ, respectively. The subunit structures and three-dimensional configurations of different classes of immunoglobulins are well known.

Antibody “effector functions” refer to those biological activities attributable to the Fc region (a native sequence Fc region or amino acid sequence variant Fc region) of an antibody. Examples of antibody effector functions include C1q binding; complement dependent cytotoxicity; Fc receptor binding; antibody-dependent cell-mediated cytotoxicity (ADCC); phagocytosis; down regulation of cell surface receptors (e.g. B cell receptor; BCR), etc.

“Antibody-dependent cell-mediated cytotoxicity” and “ADCC” refer to a cell-mediated reaction in which nonspecific cytotoxic cells that express Fc receptors (FcRs) (e.g. Natural Killer (NK) cells, neutrophils, and macrophages) recognize bound antibody on a target cell and subsequently cause lysis of the target cell. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. PNAS (USA) 95:652-656 (1998).

“Effector cells” are leukocytes which express one or more FcRs and perform effector functions. Preferably, the cells express at least FcγRIII and perform ADCC effector function. Examples of human leukocytes which mediate ADCC include peripheral blood mononuclear cells (PBMC), natural killer (NK) cells, monocytes, cytotoxic T cells and neutrophils; with PBMCs and NK cells being preferred. The effector cells may be isolated from a native source thereof, e.g. from blood or PBMCs as described herein.

The terms “Fc receptor” or “FcR” are used to describe a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (see review M. in Daëron, Annu. Rev. Immunol. 15:203-234 (1997)). FcRs are reviewed in Ravetch and Kinet, Annu. Rev. Immunol. 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., Eur. J. Immunol. 24:2429 (1994)).

“Complement dependent cytotoxicity” or “CDC” refers to the ability of a molecule to lyse a target in the presence of complement. The complement activation pathway is initiated by the binding of the first component of the complement system (C1q) to a molecule (e.g. an antibody) complexed with a cognate antigen. To assess complement activation, a CDC assay, e.g. as described in Gazzano-Santoro et al., J. Immunol. Methods 202:163 (1996), may be performed.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody which are responsible for antigen-binding. The hypervariable region generally comprises amino acid residues from a “complementarity determining region” or “CDR” (e.g. residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (e.g. residues 2632 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). “Framework Region” or “FR” residues are those variable domain residues other than the hypervariable region residues as herein defined. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-binding sites and is still capable of cross-linking antigen.

“Fv” is the minimum antibody fragment which contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the V_{H}-V_{L} dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site. The Fab fragment also contains the constant domain of the light chain and the first constant domain (CH I) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear at least one free thiol group. F(ab′)₂ antibody fragments originally were produced as pairs of Fab′ fragments which have hinge cysteines between them. Other chemical couplings of antibody fragments are also known.

The “light chains” of antibodies from any vertebrate species can be assigned to one of two clearly distinct types, called kappa (κ) and lambda (λ), based on the amino acid sequences of their constant domains.

“Single-chain Fv” or “scFv” antibody fragments comprise the V_{H} and V_{L} domains of antibody, wherein these domains are present in a single polypeptide chain. Preferably, the Fv polypeptide further comprises a polypeptide linker between the V_{H} and V_{L} domains which enables the scFv to form the desired structure for antigen binding. For a review of scFv see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a variable heavy domain (V_{H}) connected to a variable light domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

The term “triabodies” or “trivalent trimers” refers to the combination of three single chain antibodies. Triabodies are constructed with the amino acid terminus of a V_{L} or V_{H} domain, i.e., without any linker sequence. A triabody has three Fv heads with the polypeptides arranged in a cyclic, head-to-tail fashion. A possible conformation of the triabody is planar with the three binding sites located in a plane at an angle of 120 degrees from one another. Triabodies can be monospecific, bispecific or trispecific.

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. Isolated antibody includes the antibody in situ within recombinant cells since at least one component of the antibody's natural environment will not be present. Ordinarily, however, isolated antibody will be prepared by at least one purification step.

An antibody “which binds” an antigen of interest, e.g. CD59 antigen, is one capable of binding that antigen with sufficient affinity such that the antibody is useful as a therapeutic or diagnostic agent in targeting a cell expressing the antigen. Where the antibody is one which binds CD59, it will usually preferentially bind CD59 as opposed to other receptors, and does not include incidental binding such as non-specific Fc contact, or binding to post-translational modifications common to other antigens and may be one which does not significantly cross-react with other proteins. Methods, for the detection of an antibody that binds an antigen of interest, are well known in the art and can include but are not limited to assays such as FACS, cell ELISA and Western blot.

As used herein, the expressions “cell”, “cell line”, and “cell culture” are used interchangeably, and all such designations include progeny. It is also understood that all progeny may not be precisely identical in DNA content, due to deliberate or inadvertent mutations. Mutant progeny that have the same function or biological activity as screened for in the originally transformed cell are included. It will be clear from the context where distinct designations are intended.

“Treatment or treating” refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented. Hence, the mammal to be treated herein may have been diagnosed as having the disorder or may be predisposed or susceptible to the disorder.

The terms “cancer” and “cancerous” refer to or describe the physiological condition in mammals that is typically characterized by unregulated cell growth or death. Examples of cancer include, but are not limited to, carcinoma, lymphoma, blastoma, sarcoma, and leukemia or lymphoid malignancies. More particular examples of such cancers include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

A “chemotherapeutic agent” is a chemical compound useful in the treatment of cancer. Examples of chemotherapeutic agents include alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carnomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g. paclitaxel (TAXOL®, Bristol-Myers Squibb Oncology, Princeton, N.J.) and docetaxel (TAXOTERE®, Aventis, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylomithine (DMFO); retinoic acid; esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

“Mammal” for purposes of treatment refers to any animal classified as a mammal, including humans, mice, SCID or nude mice or strains of mice, domestic and farm animals, and zoo, sports, or pet animals, such as sheep, dogs, horses, cats, cows, etc. Preferably, the mammal herein is human.

“Oligonucleotides” are short-length, single- or double-stranded polydeoxynucleotides that are chemically synthesized by known methods (such as phosphotriester, phosphite, or phosphoramidite chemistry, using solid phase techniques such as described in EP 266,032, published 4 May 1988, or via deoxynucleoside H-phosphonate intermediates as described by Froehler et al., Nucl. Acids Res., 14:5399-5407, 1986. They are then purified on polyacrylamide gels.

In accordance with the present invention, “humanized” and/or “chimeric” forms of non-human (e.g. murine) immunoglobulins refer to antibodies which contain specific chimeric immunoglobulins, immunoglobulin chains or fragments thereof (such as Fv, Fab, Fab′, F(ab′)₂ or other antigen-binding subsequences of antibodies) which results in the decrease of a human anti-mouse antibody (HAMA), human anti-chimeric antibody (HACA) or a human anti-human antibody (HAHA) response, compared to the original antibody, and contain the requisite portions (e.g. CDR(s), antigen binding region(s), variable domain(s) and so on) derived from said non-human immunoglobulin, necessary to reproduce the desired effect, while simultaneously retaining binding characteristics which are comparable to said non-human immunoglobulin. For the most part, humanized antibodies are human immunoglobulins (recipient antibody) in which residues from the complementarity determining regions (CDRs) of the recipient antibody are replaced by residues from the CDRs of a non-human species (donor antibody) such as mouse, rat or rabbit having the desired specificity, affinity and capacity. In some instances, Fv framework region (FR) residues of the human immunoglobulin are replaced by corresponding non-human FR residues. Furthermore, the humanized antibody may comprise residues which are found neither in the recipient antibody nor in the imported CDR or FR sequences. These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR residues are those of a human immunoglobulin consensus sequence. The humanized antibody optimally also will comprise at least a portion of an immunoglobulin constant region (Fc), typically that of a human immunoglobulin.

“De-immunized” antibodies are immunoglobulins that are non-immunogenic, or less immunogenic, to a given species. De-immunization can be achieved through structural alterations to the antibody. Any de-immunization technique known to those skilled in the art can be employed. One suitable technique for de-immunizing antibodies is described, for example, in WO 00/34317 published Jun. 15, 2000.

An antibody which induces “apoptosis” is one which induces programmed cell death by any means, illustrated by but not limited to binding of annexin V, caspase activity, fragmentation of DNA, cell shrinkage, dilation of endoplasmic reticulum, cell fragmentation, and/or formation of membrane vesicles (called apoptotic bodies).

As used herein “antibody induced cytotoxicity” is understood to mean the cytotoxic effect derived from the hybridoma supernatant or antibody produced by the hybridoma deposited with the IDAC as accession number 280104-02, a humanized antibody of the isolated monoclonal antibody produced by the hybridoma deposited with the IDAC as accession number 280104-02, a chimeric antibody of the isolated monoclonal antibody produced by the hybridoma deposited with the IDAC as accession number 280104-02, antigen binding fragments, or antibody ligands thereof, which effect is not necessarily related to the degree of binding.

Throughout the instant specification, hybridoma cell lines, as well as the isolated monoclonal antibodies which are produced therefrom, are alternatively referred to by their internal designation, AR36A36.11.1 (murine), (ch)AR36A36.11.1 (chimeric), (hu)AR36A36.11.1 (humanized) or Depository Designation, IDAC 280104-02.

As used herein “antibody-ligand” includes a moiety which exhibits binding specificity for at least one epitope of the target antigen, and which may be an intact antibody molecule, antibody fragments, and any molecule having at least an antigen-binding region or portion thereof (i.e., the variable portion of an antibody molecule), e.g., an Fv molecule, Fab molecule, Fab′ molecule, F(ab′).sub.2 molecule, a bispecific antibody, a fusion protein, or any genetically engineered molecule which specifically recognizes and binds at least one epitope of the antigen bound by the isolated monoclonal antibody produced by the hybridoma cell line designated as IDAC 280104-02 (the IDAC 280104-02 antigen), a humanized antibody of the isolated monoclonal antibody produced by the hybridoma deposited with the IDAC as accession number 280104-02, a chimeric antibody of the isolated monoclonal antibody produced by the hybridoma deposited with the IDAC as accession number 280104-02 and antigen binding fragments.

As used herein “cancerous disease modifying antibodies” (CDMAB) refers to monoclonal antibodies which modify the cancerous disease process in a manner which is beneficial to the patient, for example by reducing tumor burden or prolonging survival of tumor bearing individuals, and antibody-ligands thereof.

A “CDMAB related binding agent”, in its broadest sense, is understood to include, but is not limited to, any form of human or non-human antibodies, antibody fragments, antibody ligands, or the like, which competitively bind to at least one CDMAB target epitope.

A “competitive binder” is understood to include any form of human or non-human antibodies, antibody fragments, antibody ligands, or the like which has binding affinity for at least one CDMAB target epitope.

Tumors to be treated include primary tumors and metastatic tumors, as well as refractory tumors. Refractory tumors include tumors that fail to respond or are resistant to treatment with chemotherapeutic agents alone, antibodies alone, radiation alone or combinations thereof. Refractory tumors also encompass tumors that appear to be inhibited by treatment with such agents but recur up to five years, sometimes up to ten years or longer after treatment is discontinued.

Tumors that can be treated include tumors that are not vascularized, or not yet substantially vascularized, as well as vascularized tumors. Examples of solid tumors, which can be accordingly treated, include breast carcinoma, lung carcinoma, colorectal carcinoma, pancreatic carcinoma, glioma and lymphoma. Some examples of such tumors include epidermoid tumors, squamous tumors, such as head and neck tumors, colorectal tumors, prostate tumors, breast tumors, lung tumors, including small cell and non-small cell lung tumors, pancreatic tumors, thyroid tumors, ovarian tumors, and liver tumors. Other examples include Kaposi's sarcoma, CNS neoplasms, neuroblastomas, capillary hemangioblastomas, meningiomas and cerebral metastases, melanoma, gastrointestinal and renal carcinomas and sarcomas, rhabdomyosarcoma, glioblastoma, preferably glioblastoma multiforme, and leiomyosarcoma.

As used herein “antigen-binding region” means a portion of the molecule which recognizes the target antigen.

As used herein “competitively inhibits” means being able to recognize and bind a determinant site to which the monoclonal antibody produced by the hybridoma cell line designated as IDAC 280104-02, (the IDAC 280104-02 antibody), a humanized antibody of the isolated monoclonal antibody produced by the hybridoma deposited with the IDAC as accession number 280104-02, a chimeric antibody of the isolated monoclonal antibody produced by the hybridoma deposited with the IDAC as accession number 280104-02, antigen binding fragments, or antibody ligands thereof, is directed using conventional reciprocal antibody competition assays. (Belanger L., Sylvestre C. and Dufour D. (1973), Enzyme linked immunoassay for alpha fetoprotein by competitive and sandwich procedures. Clinica Chimica Acta 48, 15).

As used herein “target antigen” is the IDAC 280104-02 antigen or portions thereof.

As used herein, an “immunoconjugate” means any molecule or CDMAB such as an antibody chemically or biologically linked to cytotoxins, radioactive agents, cytokines, interferons, target or reporter moieties, enzymes, toxins, anti-tumor drugs or therapeutic agents. The antibody or CDMAB may be linked to the cytotoxin, radioactive agent, cytokine, interferon, target or reporter moiety, enzyme, toxin, anti-tumor drug or therapeutic agent at any location along the molecule so long as it is able to bind its target. Examples of immunoconjugates include antibody toxin chemical conjugates and antibody-toxin fusion proteins.

Radioactive agents suitable for use as anti-tumor agents are known to those skilled in the art. For example, 131I or 211At is used. These isotopes are attached to the antibody using conventional techniques (e.g. Pedley et al., Br. J. Cancer 68, 69-73 (1993)). Alternatively, the anti-tumor agent which is attached to the antibody is an enzyme which activates a prodrug. A prodrug may be administered which will remain in its inactive form until it reaches the tumor site where it is converted to its cytotoxin form once the antibody complex is administered. In practice, the antibody-enzyme conjugate is administered to the patient and allowed to localize in the region of the tissue to be treated. The prodrug is then administered to the patient so that conversion to the cytotoxic drug occurs in the region of the tissue to be treated. Alternatively, the anti-tumor agent conjugated to the antibody is a cytokine such as interleukin-2 (IL-2), interleukin-4 (IL-4) or tumor necrosis factor alpha (TNF-α). The antibody targets the cytokine to the tumor so that the cytokine mediates damage to or destruction of the tumor without affecting other tissues. The cytokine is fused to the antibody at the DNA level using conventional recombinant DNA techniques. Interferons may also be used.

As used herein, a “fusion protein” means any chimeric protein wherein an antigen binding region is connected to a biologically active molecule, e.g., toxin, enzyme, fluorescent proteins, luminescent marker, polypeptide tag, cytokine, interferon, target or reporter moiety or protein drug.

The invention further contemplates CDMAB of the present invention to which target or reporter moieties are linked. Target moieties are first members of binding pairs. Anti-tumor agents, for example, are conjugated to second members of such pairs and are thereby directed to the site where the antigen-binding protein is bound. A common example of such a binding pair is avidin and biotin. In a preferred embodiment, biotin is conjugated to the target antigen of the CDMAB of the present invention, and thereby provides a target for an anti-tumor agent or other moiety which is conjugated to avidin or streptavidin. Alternatively, biotin or another such moiety is linked to the target antigen of the CDMAB of the present invention and used as a reporter, for example in a diagnostic system where a detectable signal-producing agent is conjugated to avidin or streptavidin.

Detectable signal-producing agents are useful in vivo and in vitro for diagnostic purposes. The signal producing agent produces a measurable signal which is detectable by external means, usually the measurement of electromagnetic radiation. For the most part, the signal producing agent is an enzyme or chromophore, or emits light by fluorescence, phosphorescence or chemiluminescence. Chromophores include dyes which absorb light in the ultraviolet or visible region, and can be substrates or degradation products of enzyme catalyzed reactions.

Moreover, included within the scope of the present invention is use of the present CDMAB in vivo and in vitro for investigative or diagnostic methods, which are well known in the art. In order to carry out the diagnostic methods as contemplated herein, the instant invention may further include kits, which contain CDMAB of the present invention. Such kits will be useful for identification of individuals at risk for certain type of cancers by detecting over-expression of the CDMAB's target antigen on cells of such individuals.

Diagnostic Assay Kits

It is contemplated to utilize the CDMAB of the present invention in the form of a diagnostic assay kit for determining the presence of a tumor. The tumor will generally be detected in a patient based on the presence of one or more tumor-specific antigens, e.g. proteins and/or polynucleotides which encode such proteins in a biological sample, such as blood, sera, urine and/or tumor biopsies, which samples will have been obtained from the patient.

The proteins function as markers which indicate the presence or absence of a particular tumor, for example a colon, breast, lung or prostate tumor. It is further contemplated that the antigen will have utility for the detection of other cancerous tumors. Inclusion in the diagnostic assay kits of binding agents comprised of CDMABs of the present invention, or CDMAB related binding agents, enables detection of the level of antigen that binds to the agent in the biological sample. Polynucleotide primers and probes may be used to detect the level of mRNA encoding a tumor protein, which is also indicative of the presence or absence of a cancer. In order for the binding assay to be diagnostic, data will have been generated which correlates statistically significant levels of antigen, in relation to that present in normal tissue, so as to render the recognition of binding definitively diagnostic for the presence of a cancerous tumor. It is contemplated that a plurality of formats will be useful for the diagnostic assay of the present invention, as are known to those of ordinary skill in the art, for using a binding agent to detect polypeptide markers in a sample. For example, as illustrated in Harlow and Lane, Antibodies: A Laboratory Manual, Cold Spring Harbor Laboratory, 1988. Further contemplated are any and all combinations, permutations or modifications of the afore-described diagnostic assay formats.

The presence or absence of a cancer in a patient will typically be determined by (a) contacting a biological sample obtained from a patient with a binding agent; (b) detecting in the sample a level of polypeptide that binds to the binding agent; and (c) comparing the level of polypeptide with a predetermined cut-off value.

In an illustrative embodiment, it is contemplated that the assay will involve the use of a CDMAB based binding agent immobilized on a solid support to bind to and remove the polypeptide from the remainder of the sample. The bound polypeptide may then be detected using a detection reagent that contains a reporter group and specifically binds to the binding agent/polypeptide complex. Illustrative detection reagents may include a CDMAB based binding agent that specifically binds to the polypeptide or an antibody or other agent that specifically binds to the binding agent, such as an anti-immunoglobulin, protein G, protein A or a lectin. In an alternative embodiment, it is contemplated that a competitive assay may be utilized, in which a polypeptide is labeled with a reporter group and allowed to bind to the immobilized binding agent after incubation of the binding agent with the sample. Indicative of the reactivity of the sample with the immobilized binding agent, is the extent to which components of the sample inhibit the binding of the labeled polypeptide to the binding agent. Suitable polypeptides for use within such assays include full length tumor-specific proteins and/or portions thereof, to which the binding agent has binding affinity.

The diagnostic kit will be provided with a solid support which may be in the form of any material known to those of ordinary skill in the art to which the protein may be attached. Suitable examples may include a test well in a microtiter plate or a nitrocellulose or other suitable membrane. Alternatively, the support may be a bead or disc, such as glass, fiberglass, latex or a plastic material such as polystyrene or polyvinylchloride. The support may also be a magnetic particle or a fiber optic sensor, such as those disclosed, for example, in U.S. Pat. No. 5,359,681.

It is contemplated that the binding agent will be immobilized on the solid support using a variety of techniques known to those of skill in the art, which are amply described in the patent and scientific literature. The term “immobilization” refers to both noncovalent association, such as adsorption, and covalent attachment, which, in the context of the present invention, may be a direct linkage between the agent and functional groups on the support, or may be a linkage by way of a cross-linking agent. In a preferred, albeit non-limiting embodiment, immobilization by adsorption to a well in a microtiter plate or to a membrane is preferable. Adsorption may be achieved by contacting the binding agent, in a suitable buffer, with the solid support for a suitable amount of time. The contact time may vary with temperature, and will generally be within a range of between about 1 hour and about 1 day.

Covalent attachment of binding agent to a solid support would ordinarily be accomplished by first reacting the support with a bifunctional reagent that will react with both the support and a functional group, such as a hydroxyl or amino group, on the binding agent. For example, the binding agent may be covalently attached to supports having an appropriate polymer coating using benzoquinone or by condensation of an aldehyde group on the support with an amine and an active hydrogen on the binding partner (see, e.g., Pierce Immunotechnology Catalog and Handbook, 1991, at A12 A13).

It is further contemplated that the diagnostic assay kit will take the form of a two-antibody sandwich assay. This assay may be performed by first contacting an antibody, e.g. the instantly disclosed CDMAB that has been immobilized on a solid support, commonly the well of a microtiter plate, with the sample, such that polypeptides within the sample are allowed to bind to the immobilized antibody. Unbound sample is then removed from the immobilized polypeptide-antibody complexes and a detection reagent (preferably a second antibody capable of binding to a different site on the polypeptide) containing a reporter group is added. The amount of detection reagent that remains bound to the solid support is then determined using a method appropriate for the specific reporter group.

In a specific embodiment, it is contemplated that once the antibody is immobilized on the support as described above, the remaining protein binding sites on the support will be blocked, via the use of any suitable blocking agent known to those of ordinary skill in the art, such as bovine serum albumin or Tween 20™ (Sigma Chemical Co., St. Louis, Mo.). The immobilized antibody would then be incubated with the sample, and polypeptide would be allowed to bind to the antibody. The sample could be diluted with a suitable diluent, such as phosphate-buffered saline (PBS) prior to incubation. In general, an appropriate contact time (i.e., incubation time) would be selected to correspond to a period of time sufficient to detect the presence of polypeptide within a sample obtained from an individual with the specifically selected tumor. Preferably, the contact time is sufficient to achieve a level of binding that is at least about 95 percent of that achieved at equilibrium between bound and unbound polypeptide. Those of ordinary skill in the art will recognize that the time necessary to achieve equilibrium may be readily determined by assaying the level of binding that occurs over a period of time.

It is further contemplated that unbound sample would then be removed by washing the solid support with an appropriate buffer. The second antibody, which contains a reporter group, would then be added to the solid support. Incubation of the detection reagent with the immobilized antibody-polypeptide complex would then be carried out for an amount of time sufficient to detect the bound polypeptide. Subsequently, unbound detection reagent would then be removed and bound detection reagent would be detected using the reporter group. The method employed for detecting the reporter group is necessarily specific to the type of reporter group selected, for example for radioactive groups, scintillation counting or autoradiographic methods are generally appropriate. Spectroscopic methods may be used to detect dyes, luminescent groups and fluorescent groups. Biotin may be detected using avidin, coupled to a different reporter group (commonly a radioactive or fluorescent group or an enzyme). Enzyme reporter groups may generally be detected by the addition of substrate (generally for a specific period of time), followed by spectroscopic or other analysis of the reaction products.

In order to utilize the diagnostic assay kit of the present invention to determine the presence or absence of a cancer, such as prostate cancer, the signal detected from the reporter group that remains bound to the solid support would generally be compared to a signal that corresponds to a predetermined cut-off value. For example, an illustrative cut-off value for the detection of a cancer may be the average mean signal obtained when the immobilized antibody is incubated with samples from patients without the cancer. In general, a sample generating a signal that is about three standard deviations above the predetermined cut-off value would be considered positive for the cancer. In an alternate embodiment, the cut-off value might be determined by using a Receiver Operator Curve, according to the method of Sackett et al., Clinical Epidemiology. A Basic Science for Clinical Medicine, Little Brown and Co., 1985, p. 106-7. In such an embodiment, the cut-off value could be determined from a plot of pairs of true positive rates (i.e., sensitivity) and false positive rates (100 percent-specificity) that correspond to each possible cut-off value for the diagnostic test result. The cut-off value on the plot that is the closest to the upper left-hand corner (i.e., the value that encloses the largest area) is the most accurate cut-off value, and a sample generating a signal that is higher than the cut-off value determined by this method may be considered positive. Alternatively, the cut-off value may be shifted to the left along the plot, to minimize the false positive rate, or to the right, to minimize the false negative rate. In general, a sample generating a signal that is higher than the cut-off value determined by this method is considered positive for a cancer.

It is contemplated that the diagnostic assay enabled by the kit will be performed in either a flow-through or strip test format, wherein the binding agent is immobilized on a membrane, such as nitrocellulose. In the flow-through test, polypeptides within the sample bind to the immobilized binding agent as the sample passes through the membrane. A second, labeled binding agent then binds to the binding agent-polypeptide complex as a solution containing the second binding agent flows through the membrane. The detection of bound second binding agent may then be performed as described above. In the strip test format, one end of the membrane to which binding agent is bound will be immersed in a solution containing the sample. The sample migrates along the membrane through a region containing second binding agent and to the area of immobilized binding agent. Concentration of the second binding agent at the area of immobilized antibody indicates the presence of a cancer. Generation of a pattern, such as a line, at the binding site, which can be read visually, will be indicative of a positive test. The absence of such a pattern indicates a negative result. In general, the amount of binding agent immobilized on the membrane is selected to generate a visually discernible pattern when the biological sample contains a level of polypeptide that would be sufficient to generate a positive signal in the two-antibody sandwich assay, in the format discussed above. Preferred binding agents for use in the instant diagnostic assay are the instantly disclosed antibodies, antigen-binding fragments thereof, and any CDMAB related binding agents as herein described. The amount of antibody immobilized on the membrane will be any amount effective to produce a diagnostic assay, and may range from about 25 nanograms to about 1 microgram. Typically such tests may be performed with a very small amount of biological sample.

Additionally, the CDMAB of the present invention may be used in the laboratory for research due to its ability to identify its target antigen.

In order that the invention herein described may be more fully understood, the following description is set forth.

The present invention provides CDMAB (i.e., IDAC 280104-02 CDMAB, a humanized antibody of the isolated monoclonal antibody produced by the hybridoma deposited with the IDAC as accession number 280104-02, a chimeric antibody of the isolated monoclonal antibody produced by the hybridoma deposited with the IDAC as accession number 280104-02, antigen binding fragments, or antibody ligands thereof) which specifically recognize and bind the IDAC 280104-02 antigen.

The CDMAB of the isolated monoclonal antibody produced by the hybridoma deposited with the IDAC as accession number 280104-02 may be in any form as long as it has an antigen-binding region which competitively inhibits the immunospecific binding of the isolated monoclonal antibody produced by hybridoma IDAC 280104-02 to its target antigen. Thus, any recombinant proteins (e.g., fusion proteins wherein the antibody is combined with a second protein such as a lymphokine or a tumor inhibitory growth factor) having the same binding specificity as the IDAC 280104-02 antibody fall within the scope of this invention.

In one embodiment of the invention, the CDMAB is the IDAC 280104-02 antibody.

In other embodiments, the CDMAB is an antigen binding fragment which may be a Fv molecule (such as a single-chain Fv molecule), a Fab molecule, a Fab′ molecule, a F(ab′)2 molecule, a fusion protein, a bispecific antibody, a heteroantibody or any recombinant molecule having the antigen-binding region of the IDAC 280104-02 antibody. The CDMAB of the invention is directed to the epitope to which the IDAC 280104-02 monoclonal antibody is directed.

The CDMAB of the invention may be modified, i.e., by amino acid modifications within the molecule, so as to produce derivative molecules. Chemical modification may also be possible. Modification by direct mutation, methods of affinity maturation, phage display or chain shuffling may also be possible.

Affinity and specificity can be modified or improved by mutating CDR and/or phenylalanine tryptophan (FW) residues and screening for antigen binding sites having the desired characteristics (e.g., Yang et al., J. Mol. Biol., (1995) 254: 392-403). One way is to randomize individual residues or combinations of residues so that in a population of otherwise identical antigen binding sites, subsets of from two to twenty amino acids are found at particular positions. Alternatively, mutations can be induced over a range of residues by error prone PCR methods (e.g., Hawkins et al., J. Mol. Biol., (1992) 226: 889-96). In another example, phage display vectors containing heavy and light chain variable region genes can be propagated in mutator strains of E. coli (e.g., Low et al., J. Mol. Biol., (1996) 250: 359-68). These methods of mutagenesis are illustrative of the many methods known to one of skill in the art.

Another manner for increasing affinity of the antibodies of the present invention is to carry out chain shuffling, where the heavy or light chain are randomly paired with other heavy or light chains to prepare an antibody with higher affinity. The various CDRs of the antibodies may also be shuffled with the corresponding CDRs in other antibodies.

Derivative molecules would retain the functional property of the polypeptide, namely, the molecule having such substitutions will still permit the binding of the polypeptide to the IDAC 280104-02 antigen or portions thereof.

These amino acid substitutions include, but are not necessarily limited to, amino acid substitutions known in the art as “conservative”.

For example, it is a well-established principle of protein chemistry that certain amino acid substitutions, entitled “conservative amino acid substitutions,” can frequently be made in a protein without altering either the conformation or the function of the protein.

Such changes include substituting any of isoleucine (I), valine (V), and leucine (L) for any other of these hydrophobic amino acids; aspartic acid (D) for glutamic acid (E) and vice versa; glutamine (Q) for asparagine (N) and vice versa; and serine (S) for threonine (T) and vice versa. Other substitutions can also be considered conservative, depending on the environment of the particular amino acid and its role in the three-dimensional structure of the protein. For example, glycine (G) and alanine (A) can frequently be interchangeable, as can alanine and valine (V). Methionine (M), which is relatively hydrophobic, can frequently be interchanged with leucine and isoleucine, and sometimes with valine. Lysine (K) and arginine (R) are frequently interchangeable in locations in which the significant feature of the amino acid residue is its charge and the differing pK's of these two amino acid residues are not significant. Still other changes can be considered “conservative” in particular environments.

### EXAMPLE 1

### In Vivo Tumor Experiment with Human PC-3 Cancer Cells

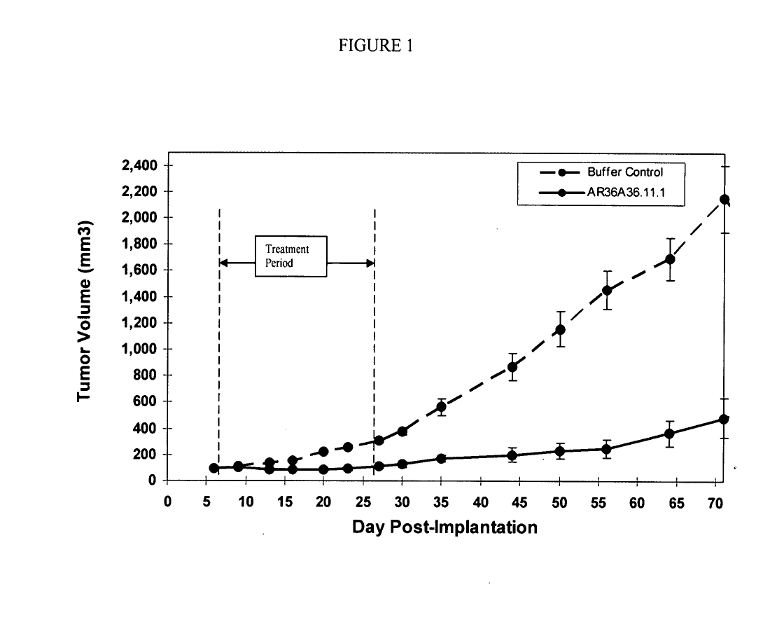

AR36A36.11.1 has previously demonstrated (as disclosed in Ser. No. 11/067,366) efficacy in a preventative in vivo model of prostate cancer. To extend this finding AR36A36.11.1 was tested in an established PC-3 prostate cancer xenograft model. With reference to FIGS. 1 and 2, 8 to 10 week old male athymic nude mice were implanted with 5 million human prostate cancer cells (PC-3) in 100 microliters PBS solution injected subcutaneously in the right flank of each mouse. The mice were randomly divided into 2 treatment groups of 10. On day 6 after implantation, when the average mouse tumor volume reached approximately 95 mm³, 20 mg/kg of AR36A36.11.1 test antibody or buffer control was administered intraperitoneally to each cohort in a volume of 300 microliters after dilution from the stock concentration with a diluent that contained 2.7 mM KCl, 1 mM KH₂PO₄, 137 mM NaCl and 20 mM Na₂HPO₄. The antibody and control samples were then administered three times per week for around 3 weeks. Tumor growth was measured every 4-10 days with calipers. The treatment was completed after 10 doses of antibody. Body weights of the animals were recorded at the same time as tumor measurement. All animals were euthanized according to CCAC guidelines at the end of the study once they had reached endpoint.

AR36A36.11.1 significantly inhibited tumor growth in the PC-3 in vivo established model of human prostate cancer. Treatment with ARIUS antibody AR36A36.11.1 reduced the growth of PC-3 tumors by 81.1 percent (p=0.0004084, t-test), compared to the buffer-treated group, as determined on day 71, 44 days after the last dose of antibody (FIG. 1). Tumor growth inhibition was calculated after subtracting the initial tumor volume for both the control and treatment groups.

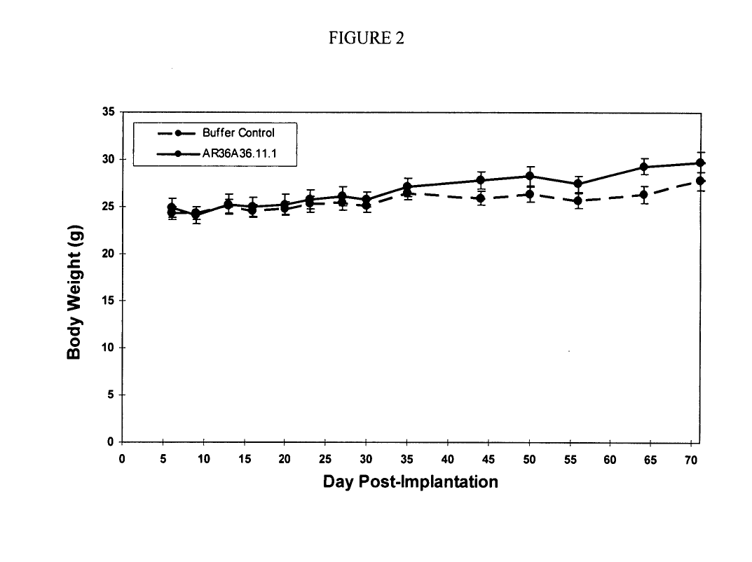

There were no obvious clinical signs of toxicity throughout the study. Body weight measured at weekly intervals was a surrogate for well-being and failure to thrive. The mean body weight increased in all groups over the duration of the study (FIG. 2). The mean weight gain between day 6 and day 71 was 3.47 g (14.3 percent) in the control group and 4.93 g (19.8 percent) in the AR36A36.11.1-treated group. There was no significant difference between the groups during the treatment period.

In summary, AR36A36.11.1 was well-tolerated and significantly inhibited the tumor growth in this established xenograft model of human prostate cancer.

### EXAMPLE 2

### In Vivo Tumor Experiment with Human MDA-MB-468 Breast Cancer Cells

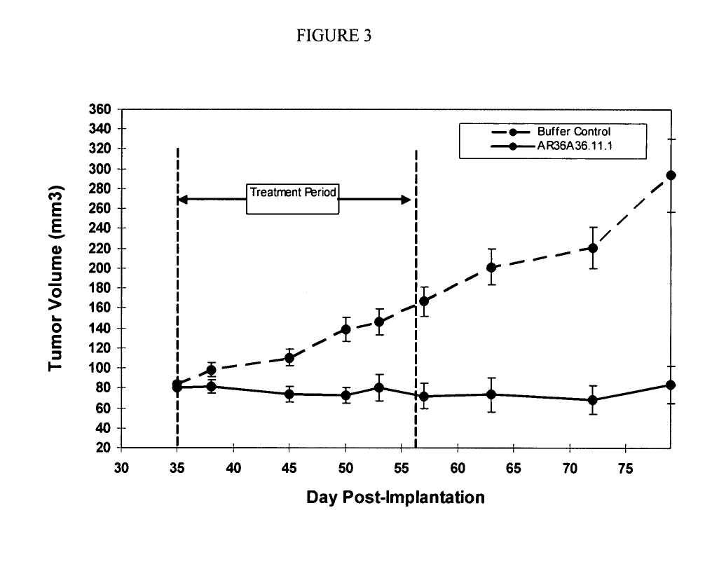

AR36A36.11.1 has previously demonstrated (as disclosed in Ser. No. 11/067,366) efficacy in a MDA-MB-231 human breast cancer xenograft model. To extend this finding to another human breast cancer model, AR36A36.11.1 was tested in an established MDA-MB-468 human breast cancer xenograft model. With reference to FIGS. 3 and 4, 8 to 10 week old female athymic nude mice were implanted with 5 million human breast cancer cells (MDA-MB-468) in 100 microliters PBS solution injected subcutaneously in the right flank of each mouse. The mice were randomly divided into 2 treatment groups of 10. On day 35 after implantation when the average mouse tumor volume reached approximately 83 mm³, 20 mg/kg of AR36A36.11.1 test antibody or buffer control was administered intraperitoneally to each cohort in a volume of 300 microliters after dilution from the stock concentration with a diluent that contained 2.7 mM KCl, 1 mM KH₂PO₄, 137 mM NaCl and 20 mM Na₂HPO₄. The antibody and control samples were then administered three times per week for around 3 weeks. Tumor growth was measured once per week with calipers. The treatment was completed after 10 doses of antibody. Body weights of the animals were recorded at the same time as tumor measurement. All animals were euthanized according to CCAC guidelines at the end of the study once they had reached endpoint.

AR36A36.11.1 significantly inhibited tumor growth in the MDA-MB-468 in vivo established model of human breast cancer. Treatment with ARIUS antibody AR36A36.11.1 reduced the growth of MDA-MB-468 tumors by 98.6 percent (p=0.000147, t-test), compared to the buffer-treated group, as determined on day 79, 26 days after the last dose of antibody (FIG. 3). Tumor growth inhibition was calculated after subtracting the initial tumor volume for both the control and treatment groups.

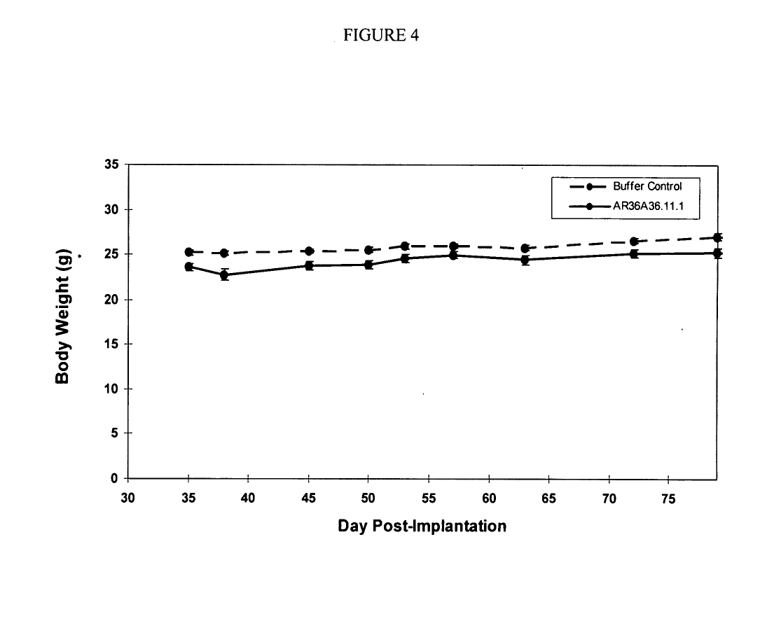

There were no obvious clinical signs of toxicity throughout the study. Body weight measured at weekly intervals was a surrogate for well-being and failure to thrive. The mean body weight increased in all groups over the duration of the study (FIG. 4). The mean weight gain between day 35 and day 79 was 1.82 g (7.2 percent) in the control group and 1.59 g (6.7 percent) in the AR36A36.11.1-treated group. There was no significant difference between the groups during the treatment period.

In summary, AR36A36.11.1 was well-tolerated and significantly inhibited the tumor growth in another human breast cancer xenograft model.

### EXAMPLE 3

### In Vivo Tumor Experiment with Human MDA-MB-231 Breast Cancer Cells

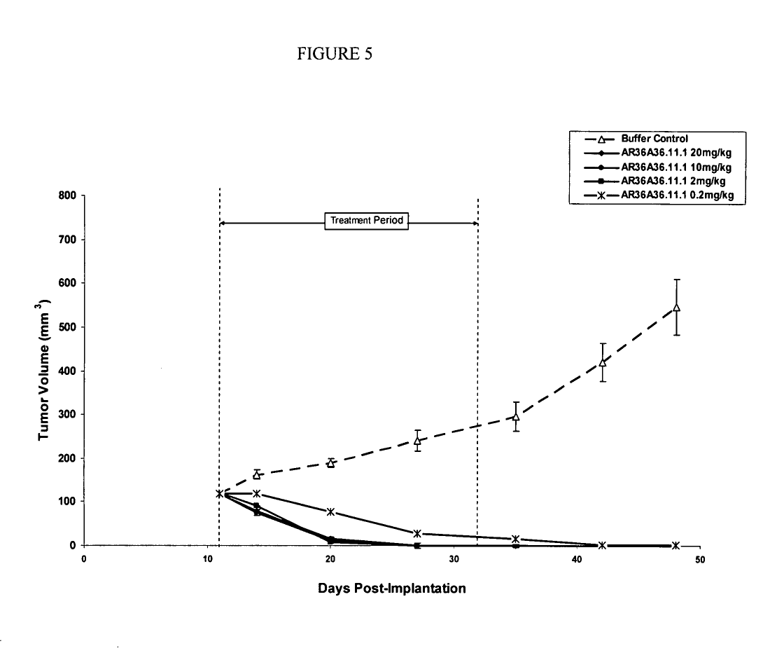

AR36A36.11.1 has previously demonstrated (as disclosed in Ser. No. 11/067,366) efficacy in an established MDA-MB-231 human breast cancer xenograft model. To determine effective dose levels, AR36A36.11.1 was tested at various doses in an established MDA-MB-31 human breast cancer xenograft model. With reference to FIGS. 5 and 6, 8 to 10 week old female SCID mice were implanted with 5 million human breast cancer cells (MDA-MB-231) in 100 microliters PBS solution injected subcutaneously in the right flank of each mouse. The mice were randomly divided into 5 treatment groups of 10 when the average mouse tumor volume reached approximately 100 mm³. On day 11 after implantation, 20, 10, 2 or 0.2 mg/kg of AR36A36.11.1 test antibody or buffer control was administered intraperitoneally to each cohort in a volume of 300 microliters after dilution from the stock concentration with a diluent that contained 2.7 mM KCl, 1 mM KH₂PO₄, 137 mM NaCl and 20 mM Na₂HPO₄. The antibody and control samples were then administered three times per week for around 3 weeks. Tumor growth was measured once every 4-7 day with calipers. The treatment was completed after 10 doses of antibody. Body weights of the animals were recorded at the same time as tumor measurements. All animals were euthanized according to CCAC guidelines at the end of the study once they had reached endpoint.

AR36A36.11.1 demonstrated dose-dependent tumor growth inhibition and regression in the MDA-MB-231 in vivo established model of human breast cancer at the lowest dose of 0.2 mg/kg during the treatment period. Tumor growth regression was also maintained, with the lowest dose, after treatment. Treatment with ARIUS antibody AR36A36.11.1 at doses of 20, 10 and 2 mg/kg completely eradicated the growth of MDA-MB-231 tumors by 100 percent (p<0.00001, t-test), and treatment at dose 0.2 mg/kg by 98 percent (p<0.0001), compared to the buffer-treated group, as determined on day 48, 16 days after last dose of antibody (FIG. 5).

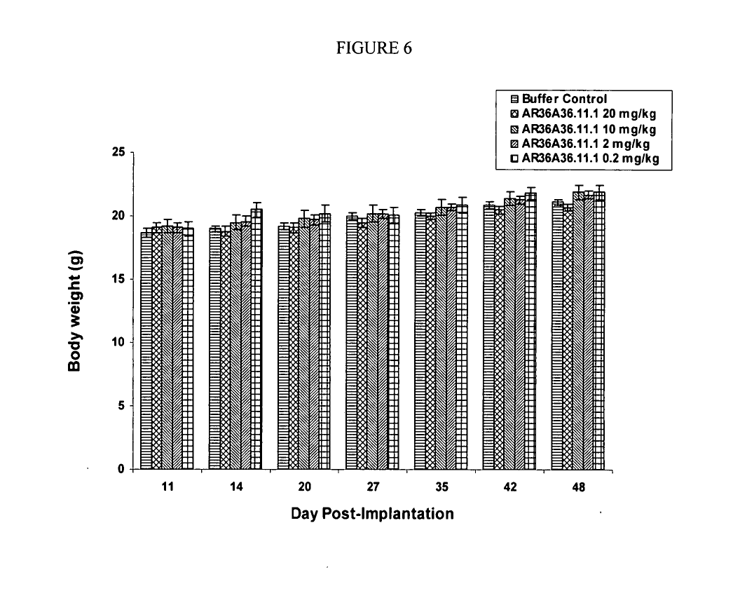

There were no obvious clinical signs of toxicity throughout the study. Body weight measured at 4-7 day intervals was a surrogate for well being and failure to thrive. The mean body weight increased in all groups over the duration of the study (FIG. 6). The mean weight gain between day 11 and day 48 was 2.5 g (13.4 percent) in the control group and 1.6 g (8.4 percent), 2.7 g (14.1 percent), 2.6 g (13.6 percent), and 2.9 g (15.3 percent) in the AR36A36.11.1-treated group at doses of 20, 10, 2 and 0.2 mg/kg, respectively. There was no significant difference between the groups during the treatment period.

In summary, AR36A36.11.1 was well-tolerated and demonstrated dose-dependent significant tumor growth inhibition and regression in this human breast cancer xenograft model with significant efficacy still being demonstrated at the lowest dose of 0.2 mg/kg.

### EXAMPLE 4

### In Vivo Tumor Experiment with Human NCI-H520 Lung Cancer Cells

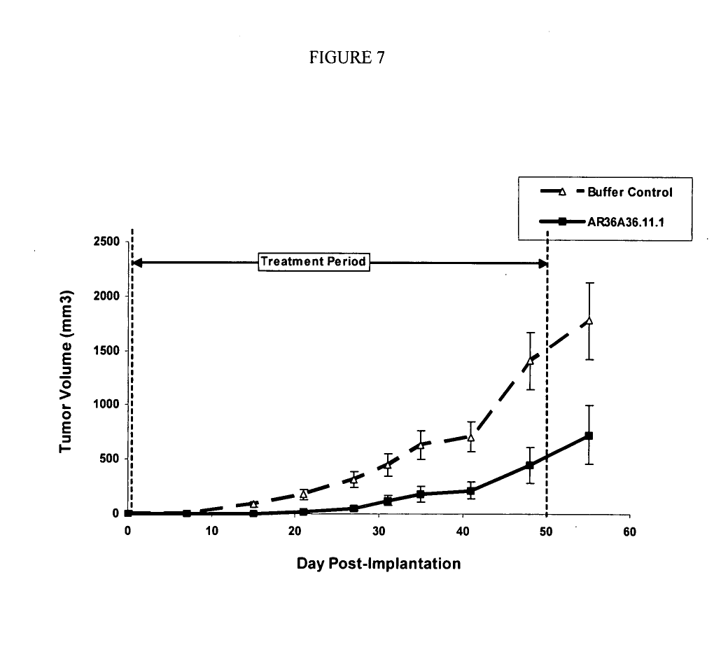

AR36A36.11.1 has previously demonstrated (as disclosed in Ser. No. 11/067,366) efficacy in human breast, prostate and colon cancer xenograft models. To demonstrate efficacy in a lung cancer model, AR36A36.11.1 was tested in a NCI-H520 human lung squamous cell carcinoma xenograft model. With reference to FIGS. 7, 8 and 9, 8 to 10 week old female SCID mice were implanted with 5 million human squamous cell lung carcinoma cells (NCI-H520) in 100 microliters PBS solution injected subcutaneously in the right flank of each mouse. The mice were randomly divided into 2 treatment groups of 10. One day after implantation, 20 mg/kg of AR36A36.11.1 test antibody or buffer control was administered intraperitoneally to each cohort in a volume of 300 microliters after dilution from the stock concentration with a diluent that contained 2.7 mM KCl, 1 mM KH₂PO₄, 137 mM NaCl and 20 mM Na₂HPO₄. The antibody and control samples were then administered once per week for 7 weeks. Tumor growth was measured once per week with calipers. The treatment was completed after 8 doses of antibody. Body weights of the animals were recorded at the same time as tumor measurement. All animals were euthanized according to CCAC guidelines at the end of the study once they had reached endpoint.

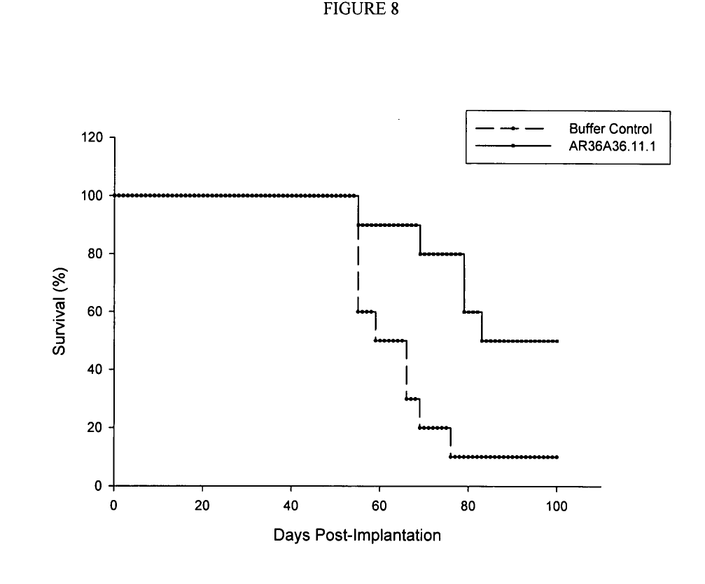

AR36A36.11.1 significantly inhibited tumor growth in the NCI-H520 in vivo prophylactic model of human lung squamous cell carcinoma. Treatment with ARIUS antibody AR36A36.11.1 reduced the growth of NCI-H520 tumors by 58.9 percent (p=0.03113, t-test), compared to the buffer-treated group, as determined on day 55, 5 days after the last dose of antibody (FIG. 7). The study was continued and survival was monitored until day 100, 50 days after the last dose, when 90 percent (9/10) of the mice in the control group had been removed from the study due to reaching endpoint. However, 50 percent (5/10) of the mice in the AR36A36.11.1-treated group were still alive (FIG. 8) at that time point.

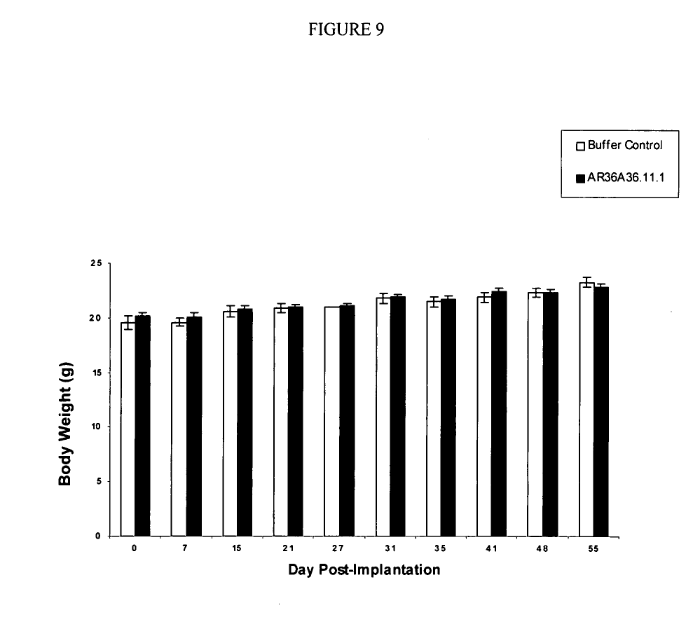

There were no obvious clinical signs of toxicity throughout the study. Body weight measured at weekly intervals was a surrogate for well-being and failure to thrive. The mean body weight increased in all groups over the duration of the study (FIG. 9). The mean weight gain between day 0 and day 55 was 3.7 g (18.9 percent) in the control group and 2.6 g (12.9 percent) in the AR36A36.11.1-treated group. There was no significant difference between the groups during the treatment period.

In summary, AR36A36.11.1 was well-tolerated and significantly inhibited tumor growth and increased survival in this human lung squamous cell carcinoma xenograft model. AR36A36.11.1 has demonstrated efficacy against four different human cancer indications: lung squamous cell, prostate, breast and colon. Treatment benefits were observed in several well-recognized models of human cancer disease suggesting pharmacologic and pharmaceutical benefits of this antibody for therapy in other mammals, including man. In toto, this data demonstrates that the AR36A36.11.1 antigen is a cancer associated antigen and is expressed on human cancer cells, and is a pathologically relevant cancer target.

### EXAMPLE 5

### Cross Competition Experiments

In order to further characterize the binding properties of AR36A36.11.1, antibody competition experiments were carried out with 10A304.7 (another previously disclosed anti-CD59 antibody; Ser. No. 10/413,755 now U.S. Pat. No. 6,794,494, Ser. No. 10/944,664 and Ser. No. 11/361,153). Western blots were done to determine if 10A304.7 and AR36A36.11.1 recognize similar or distinct epitopes of CD59. Five hundred micrograms of an MDA-MB-231 total membrane preparation was subjected to SDS-PAGE under non-reducing conditions using preparative well combs that spanned the entire length of each of two 10 percent polyacrylamide gels. The proteins from the gels were transferred to PVDF membranes at 150V for 2 hours at 4° C. The membranes were blocked with 5 percent skim milk in TBST for approximately 17 hours at 4° C. on a rotating platform. The membranes were washed twice with approximately 20 mL of TBST and were placed in a Western multiscreen apparatus creating twenty separate channels in which different probing solutions were applied. Previously, biotinylated 10A304.7 and AR36A36.11.1 had been prepared using EZ-Link NHS-PEO Solid Phase Biotinylation Kit (Pierce, Rockford, Ill.). Primary antibody solutions were prepared by mixing biotinylated 10A304.7 or biotinylated AR36A36.11.1 with varying concentrations of non-biotinylated antibodies. Specifically, solutions were prepared containing 0.05 micrograms/mL of biotinylated AR36A36.11.1 in 3 percent skim milk in TBST plus 0.5 micrograms/mL, 5 micrograms/mL, 50 micrograms/mL, 500 micrograms/mL or 1000 micrograms/mL of non-biotinylated antibody. The non-biotinylated antibodies that were used were AR36A36.11.1, 10A304.7 and control antibody 8B1B.1 (anti-bluetongue virus; IgG2b, kappa, purified in-house). Solutions containing 0.05 micrograms/mL of biotinylated 10A304.7 were prepared with the same concentrations listed above of the non-biotinylated antibodies 10A304.7, AR36A36.11.1 and control antibody 8A3B.6 (anti-bluetongue virus; IgG2a, kappa, purified in-house). A negative control solution consisting of three percent skim milk in TBST was added to two channels on each membrane.

The primary antibody solutions were incubated in separate channels on the membranes for 2 hours at room temperature on a rocking platform. Each channel was washed 3 times with TBST for 10 minutes on a rocking platform. Secondary solution consisting of 0.01 microgram/mL peroxidase conjugated streptavidin (Jackson Immunoresearch, West Grove, Pa.) in 3 percent skim milk in TBST was applied to each channel on the membrane, except for one channel on each membrane to which 3 percent skim milk in TBST alone was applied as a negative control. The membranes were incubated in secondary solution for 1 hour at room temperature on a rocking platform. Each channel was washed 3 times with TBST for 10 minutes on a rocking platform. The membranes were removed from the multiscreen apparatus and incubated with an enhanced chemiluminescence detection solution (GE Healthcare, Life Sciences formerly Amersham Biosciences, Piscataway, N.J.) according to manufacturer's directions. The membranes were then exposed to film and developed.

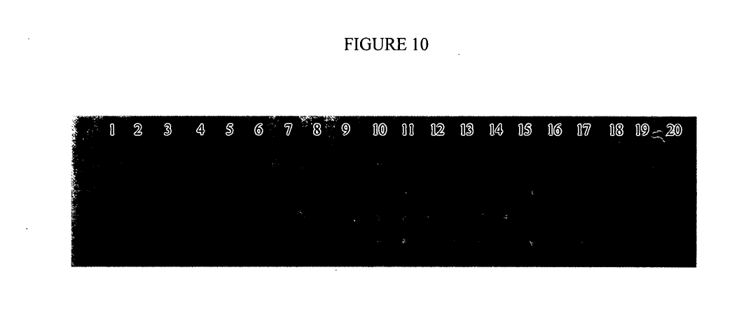

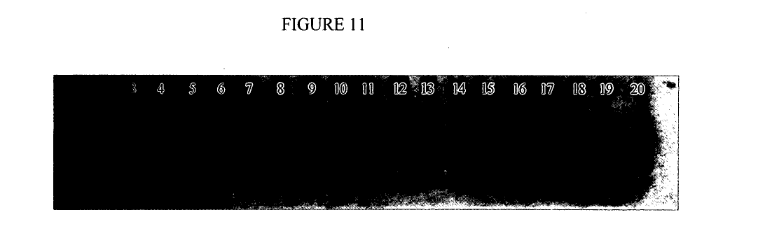

FIGS. 10 and 11 show the results of the antibody competition experiments. Binding of biotinylated AR36A36.11.1 was completely inhibited when mixed with non-biotinylated AR36A36.11.1 at concentrations of 5 micrograms/mL and greater (100× excess; FIG. 10 lanes 3-7) while the binding of biotinylated 10A304.7 was completely inhibited when mixed with non-biotinylated 10A304.7 at concentrations of 50 micrograms/mL and greater (1000× excess; FIG. 11 lanes 3-7). The binding of biotinylated AR36A36.11.1 was not inhibited in any of the samples containing IgG2b isotype control antibody (FIG. 10 lanes 15-19) and the binding of biotinylated 10A304.7 was not inhibited in any of the samples containing IgG2a isotype control antibody (FIG. 11 lanes 15-19). This indicates that the inhibition of binding observed with the biotinylated antibodies mixed with the same non-biotinylated antibody was due to the occupation of antigen binding sites by the non-biotinylated antibody, not by non-specific interactions of excess antibody alone. The binding of biotinylated AR36A36.11.1 was completely inhibited when mixed with non-biotinylated 10A304.7 at concentrations of 500 micrograms/mL and higher (10000× excess; FIG. 10 lanes 9-13), and the binding of biotinylated 10A304.7 was completely inhibited when mixed with non-biotinylated AR36A36.11.1 at all concentrations tested (FIG. 11 lanes 9-13). These results indicate that the binding of AR36A36.11.1 prevents the binding of 10A304.7 and vice versa. Overall, the results of the competition Western blots suggest that the epitopes of the CD59 molecule that are recognized by AR36A36.11.1 and 10A304.7 are similar to each other.

### EXAMPLE 6

### Epitope Mapping

Epitope mapping experiments were carried out in order to determine the region(s) of the CD59 molecule that were recognized by 10A304.7 (another previously disclosed anti-CD59 antibody; Ser. No. 11/361,153) and AR36A36.11.1. Overlapping 15-mer peptides were synthesized based on the amino acid sequence of CD59 using standard Fmoc-chemistry and deprotected using trifluoric acid with scavengers. Additionally, up to 30-mer double-looped, triple-looped and sheet-like peptides were synthesized on chemical scaffolds in order to reconstruct discontinuous epitopes of the CD59 molecule, using Chemically Linked Peptides on Scaffolds (CLIPS) technology. The looped peptides were synthesized containing a dicysteine, which was cyclized by treating with alpha,alpha′-dibromoxylene and the size of the loop was varied by introducing cysteine residues at variable spacing. If other cysteines besides the newly introduced cysteines were present, they were replaced by an alanine. The side-chains of the multiple cysteines in the peptides were coupled to CLIPS templates by reacting onto credit-card format polypropylene PEPSCAN cards (455 peptide formats/card) with a 0.5 mM solution of CLIPS template such as 1,3-bis(bromomethyl)benzene in ammonium bicarbonate (20 mM, pH 7.9)/acetonitrile (1:1(v/v)). The cards were gently shaken in the solution for 30 to 60 minutes while completely covered in solution. Finally, the cards were washed extensively with excess of H₂O and sonicated in disrupt-buffer containing 1 percent SDS/0.1 percent beta-mercaptoethanol in PBS (pH 7.2) at 70° C. for 30 minutes, followed by sonication in H₂O for another 45 minutes. In total, 3811 different peptides were synthesized. The binding of antibody to each peptide was tested in a PEPSCAN-based ELISA. The 455-well credit card format polypropylene cards containing the covalently linked peptides were incubated with primary antibody solution consisting of 10 micrograms/mL of either 10A304.7 or AR36A36.11.1 diluted in blocking solution (5 percent ovalbumin (w/v) in PBS) overnight. After washing, the peptides were incubated with a 1/1000 dilution of rabbit anti-mouse antibody peroxidase for one hour at 25° C. After washing, the peroxidase substrate 2,2′-azino-di-3-ethylbenzthiazoline sulfonate (ABTS) and 2 microlitres of 3 percent H₂O₂ were added. After one hour, the color development was measured. The color development was quantified with a charge coupled device (CCD)-camera and an image processing system.

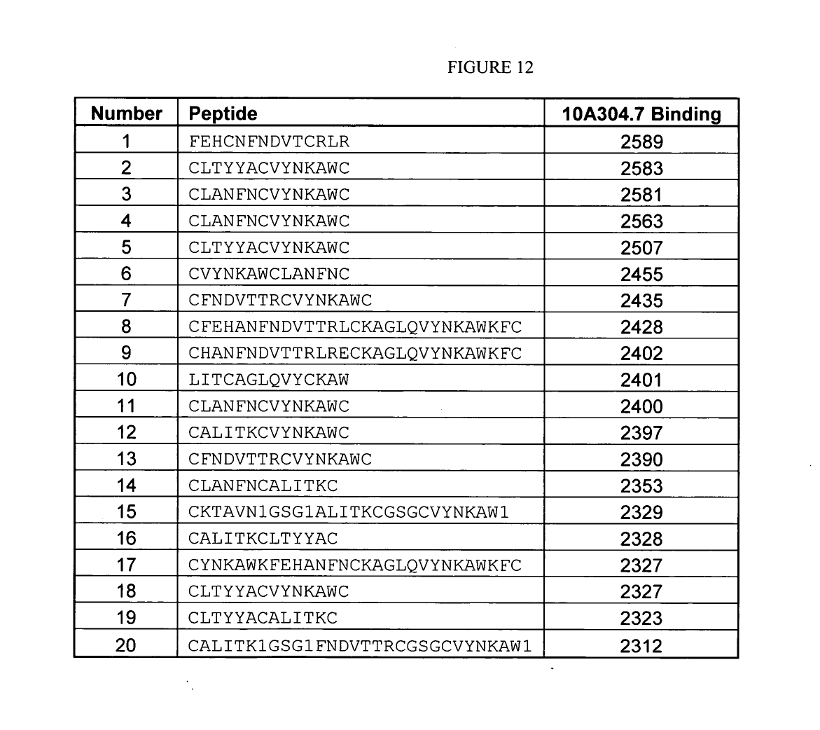

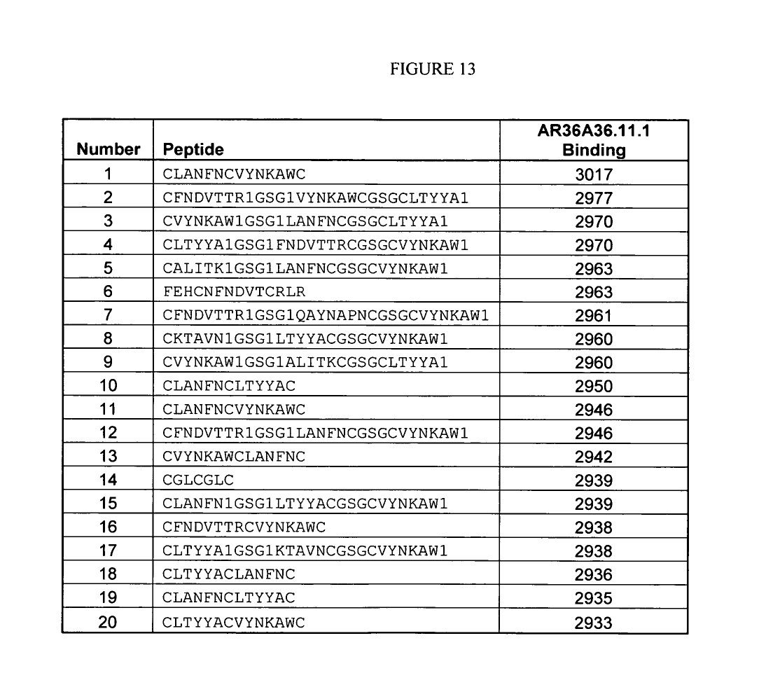

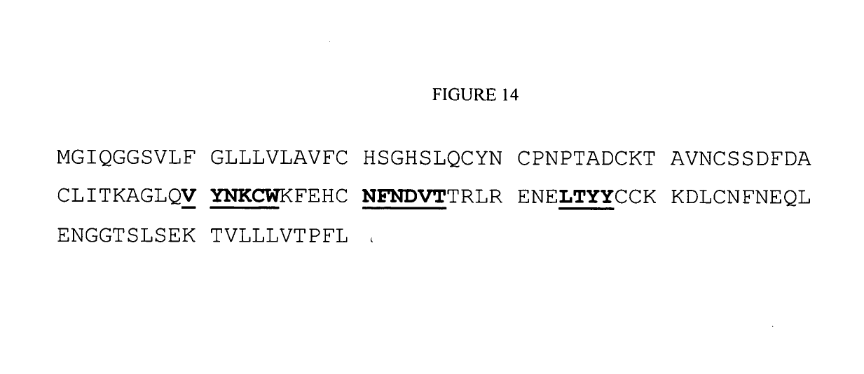

The twenty peptides (out of 3811) to which 10A304.7 and AR36A36.11.1 bound most strongly are listed in FIGS. 12 and 13, respectively. Three amino acid hotspot regions (VYNKCW, NFNDVT and LTYY) were identified for both 10A304.7 and AR36A36.11.1 by analyzing the composition of the peptides to which both antibodies bound. Various combinations of the sequences VYNKCW, NFNDVT and LTYY are present in 17 of the top 20 highest binding peptides for 10A304.7 and 16 of the top 20 highest binding peptides for AR36A36.11.1. The position of these amino acid sequences within the entire CD59 molecule amino acid sequence is presented in FIG. 14. Overall, these results indicate that 10A304.7 and AR36A36.11.1 recognize a similar discontinuous epitope of three parts contained within the sequence VYNKCWKFEHCNFNDVTTRLRENELTYY of CD59.

### EXAMPLE 7

### Humanization of AR36A36.11.1

Recombinant DNA techniques were performed using methods well known in the art and, as appropriate, supplier instructions for use of enzymes used in these methods. Detailed laboratory methods are also described below.

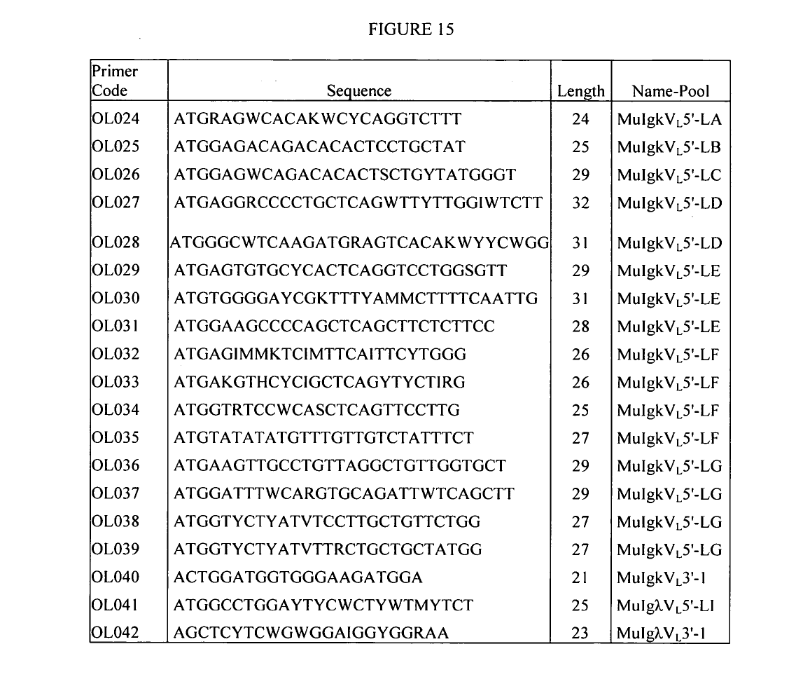

mRNA was extracted from the hybridoma AR36A36.11.1 cells using a Poly A Tract System 1000 mRNA extraction kit: (Promega Corp., Madison, Wis.) according to manufacturer's instructions. mRNA was reverse transcribed as follows: For the kappa light chain, 5.0 microliters of mRNA was mixed with 1.0 microliter of 20 pmol/microliter MuIgGκV_{L}-3′ primer OL040 (FIG. 15) and 5.5 microliters nuclease free water (Promega Corp., Madison, Wis.). For the lambda light chain, 5.0 microliters of mRNA was mixed with 1.0 microliter of 20 pmol/microliter MuIgGλV_{L}-3′ primer OL042 (FIG. 15) and 5.5 microliters nuclease free water (Promega Corp., Madison, Wis.). For the gamma heavy chain, 5 microliters of mRNA was mixed with 1.0 microliter of 20 pmol/microliter MuIgGV_{H}-3′ primer OL023 (Table 1) and 5.5 microliters nuclease free water (Promega Corp., Madison, Wis.). All three reaction mixes were placed in the pre-heated block of the thermal cycler set at 70° C. for 5 minutes. These were chilled on ice for 5 minutes before adding to each 4.0 microliters ImPromII 5× reaction buffer (Promega Corp., Madison, Wis.), 0.5 microliters RNasin ribonuclease inhibitor (Promega Corp., Madison, Wis.), 2.0 microliters 25 mM MgCl₂ (Promega Corp., Madison, Wis.), 1.0 microliter 10 mM dNTP mix (Invitrogen, Paisley, UK) and 1.0 microliter Improm II reverse transcriptase (Promega Corp., Madison, Wis.). The reaction mixes were incubated at room temperature for 5 minutes before being transferred to a pre-heated PCR block set at 42° C. for 1 hour. After this time the reverse transcriptase was heat inactivated by incubating at 70° C. in a PCR block for fifteen minutes.

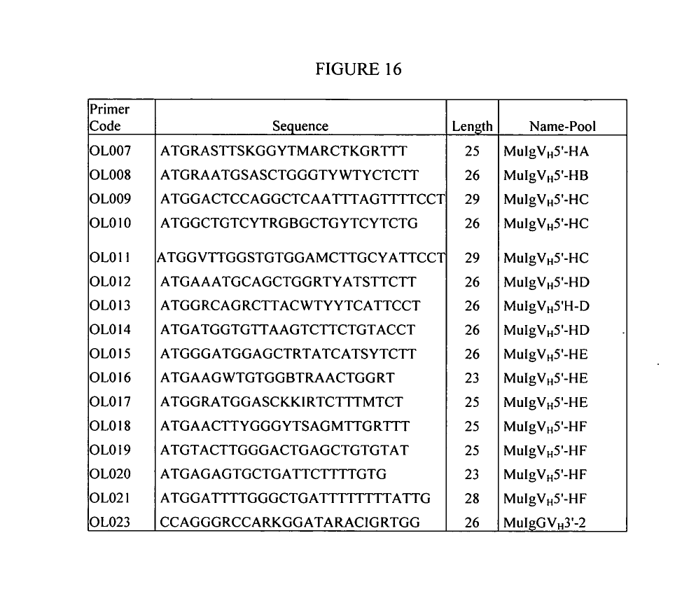

Heavy and light chain sequences were amplified from cDNA as follows: A PCR master mix was prepared by adding 37.5 microliters 10× Hi-Fi Expand PCR buffer: (Roche, Mannheim, Germany), 7.5 microliters 10 mM dNTP mix (Invitrogen, Paisley, UK) and 3.75 microliters Hi-Fi Expand DNA polymerase (Roche, Mannheim, Germany) to 273.75 microliters nuclease free water. This master mix was dispensed in 21.5 microliters aliquots into 15 thin walled PCR reaction tubes on ice. Into six of these tubes was added 2.5 microliters of MuIgVH-3′ reverse transcription reaction mix and 1.0 microliters of heavy chain 5′ primer pools HA to HF (see FIG. 16 for primer sequences and primer pool constituents). To another seven tubes was added 2.5 microliters of MuIgKVL-3′ reverse transcription reaction and 1.0 microliter of light chain 5′ primer pools LA to LG (FIG. 15). Into the final tube was added 2.5 microliters of MuIgKVL-3′ reverse transcription reaction and 1.0 microliter of lambda light chain primer MuIgλVL5′-L1. Reactions were placed in the block of the thermal cycler and heated to 95° C. for 2 minutes. The polymerase chain reaction (PCR) reaction was performed for 40 cycles of 94° C. for 30 seconds, 55° C. for 1 minute and 72° C. for 30 seconds. Finally the PCR products were heated at 72° C. for 5 minutes, and then held at 4° C.

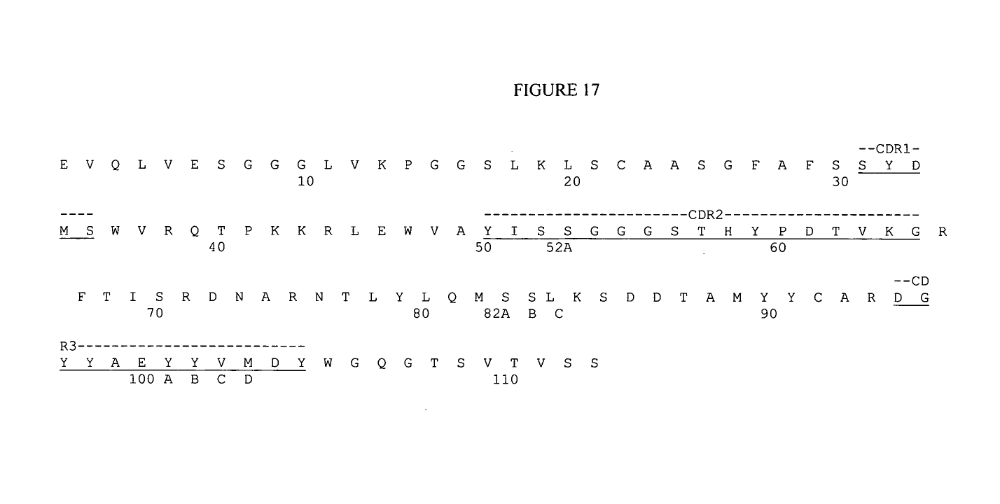

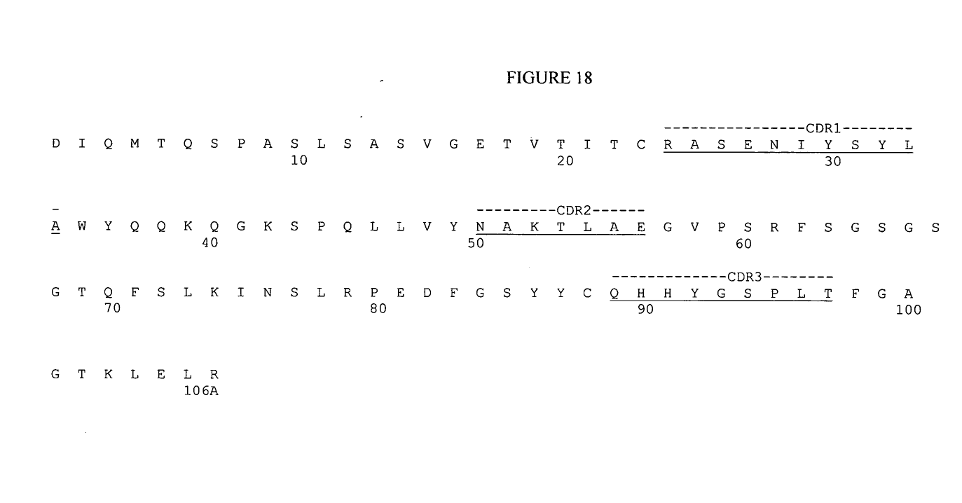

Amplification products were cloned into the pGEM-T easy vector using the pGEM-T easy Vector System I (Promega Corp., Madison, Wis.) kit and sequenced. The resultant VH and VL sequences are shown in FIGS. 17 and 18 respectively.

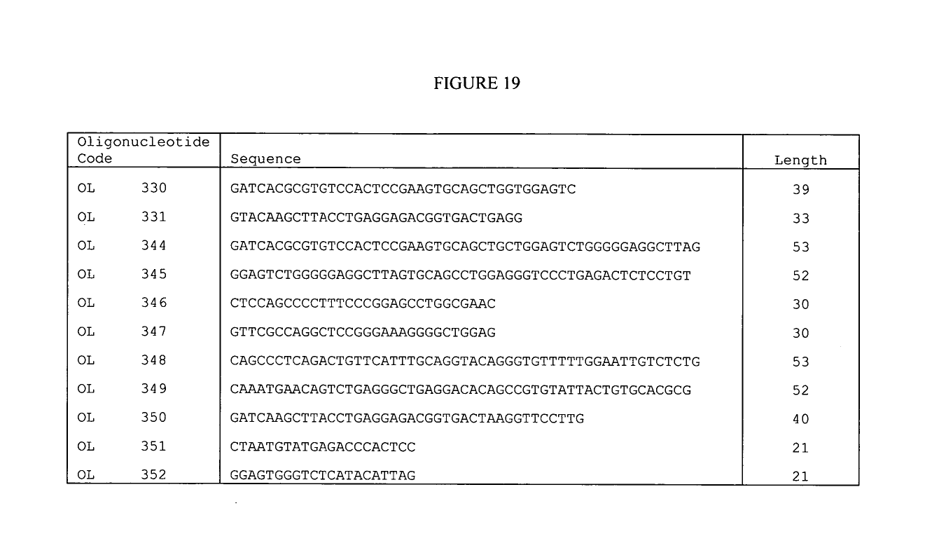

For generation of a chimeric antibody, VH region genes were amplified by PCR using the primers OL330 and OL331 (FIG. 19); these were designed to engineer in a 5′ MluI and a 3′ HindIII restriction enzyme site using plasmid DNA from one of the cDNA clones as template. Into a 0.5 mL PCR tube was added 5 microliters 10× Hi-Fi Expand PCR buffer (Roche, Mannheim, Germany), 1.0 microliter 10 mM dNTP mix (Invitrogen, Paisley, UK), 0.5 microliters of Primer OL330, 0.5 microliters of primer OL331, 1.0 microliter template DNA and 0.5 microliters Hi-Fi Expand DNA polymerase (Roche, Mannheim, Germany) to 41.5 microliters nuclease free water.

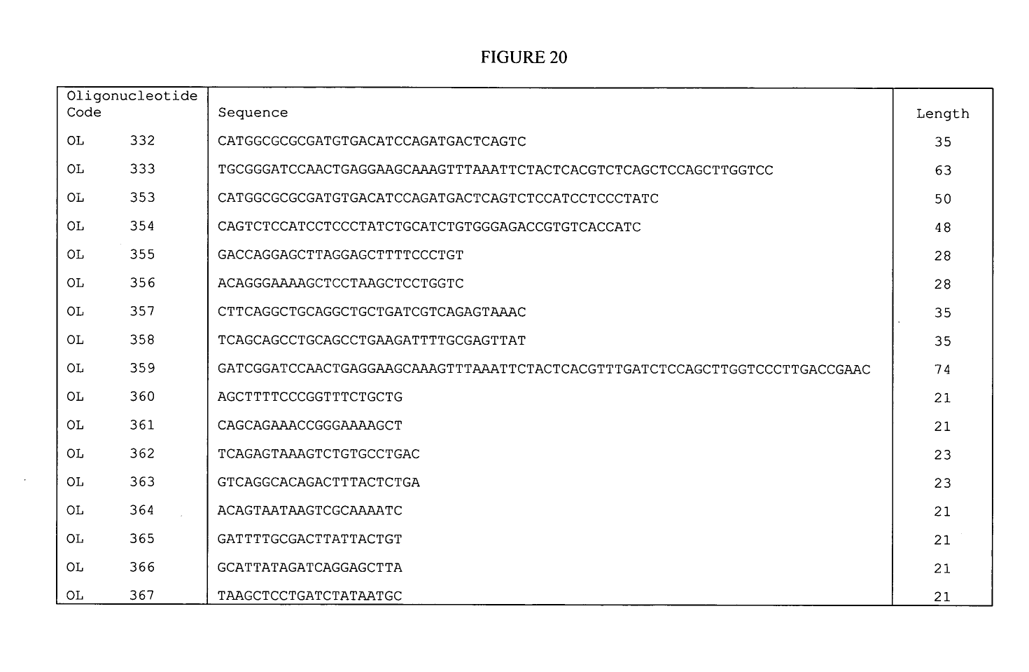

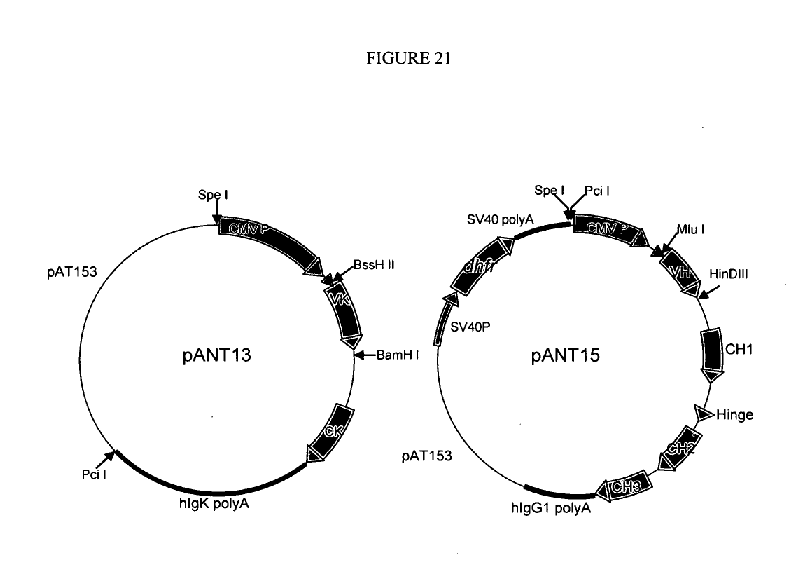

VL regions were amplified in a similar method using the oligonucleotides OL332 and OL333 (FIG. 20) to engineer in BssHII and BamHI restriction enzyme sites. Reactions were placed in the block of the thermal cycler and heated to 95° C. for 2 minutes. The polymerase chain reaction (PCR) reaction was performed for 30 cycles of 94° C. for 30 seconds, 55° C. for 1 minute and 72° C. for 30 seconds. Finally the PCR products were heated at 72° C. for 5 minutes, and then held at 4° C. VH and VL region PCR products were then cloned into the vectors pANT15 and pANT13 respectively (FIG. 21) at the MluI/HindIII and BssHII/BamHI sites respectively. Both pANT15 and pANT13 are pAT153-based plasmids containing a human Ig expression cassette. The heavy chain cassette in pANT15 consists of a human genomic IgG1 constant region gene driven by hCMVie promoter, with a downstream human IgG polyA region. pANT15 also contains a hamster dhfr gene driven by the SV40 promoter with a downstream SV40 polyA region. The light chain cassette of pANT13 is comprised of the genomic human kappa constant region driven by the hCMVie promoter with a downstream light chain polyA region. Cloning sites between a human Ig leader sequence and the constant regions allows for the insertion of the variable region genes.

NS0 cells (ECACC 85110503, Porton, UK) were co-transfected with these two plasmids via electroporation and selected in DMEM (Invitrogen, Paisley, UK) plus 5 percent FBS (Ultra low IgG Cat No. 16250-078 Invitrogen, Paisley, UK) plus Penicillin/Streptomycin (Invitrogen, Paisley, UK) plus 100 nM Methotrexate (Sigma, Poole, UK). Methotrexate resistant colonies were isolated and antibody was purified by Protein A affinity chromatography using a 1 mL HiTrap MabSelect SuRe column (GE Healthcare, Amersham, UK) following the manufacturers recommended conditions.

The chimeric antibody was tested in an ELISA-based competition assay using AR36A36.11.1 mouse antibody that was biotinylated using Biotintag micro biotinylation kit (Sigma, Poole, UK). Biotinylated mouse AR36A36.11.1 was used to bind to MDA-MB-231 cells in the presence of varying concentrations of competing antibody. MDA-MB-231 cells were cultured to near confluence in tissue culture treated, flat bottomed, 96 well plates and then fixed. Biotinylated mouse AR36A36.11.1 antibody was diluted to 1 microgram/mL and mixed with equal volumes of competing antibody at concentrations ranging from 0 to 5 micrograms/mL. 100 microliters of the antibody mixes were transferred into the wells of the MDA-MB-231 coated plate and this was incubated at room temperature for 1 hour. The plate was washed and bound biotinylated mouse AR36A36.11.1 was detected by adding a strepavidin-HRP conjugate (Sigma, Poole, UK) (diluted at 1:500) and OPD substrate (Sigma, Poole, UK). The assay was developed in the dark for 5 minutes before being stopped by the addition of 3 M HCl. The assay plate was then read in a MRX TCII plate reader (Dynex Technologies, Worthing, UK) at absorbance 490 nm. The chimeric antibody ((ch)AR36A36.11.1) was shown to be equivalent to the mouse AR36A36.11.1 antibody in competing with biotinylated AR36A36.11.1 antibody for binding to MDA-MB-231 cells.

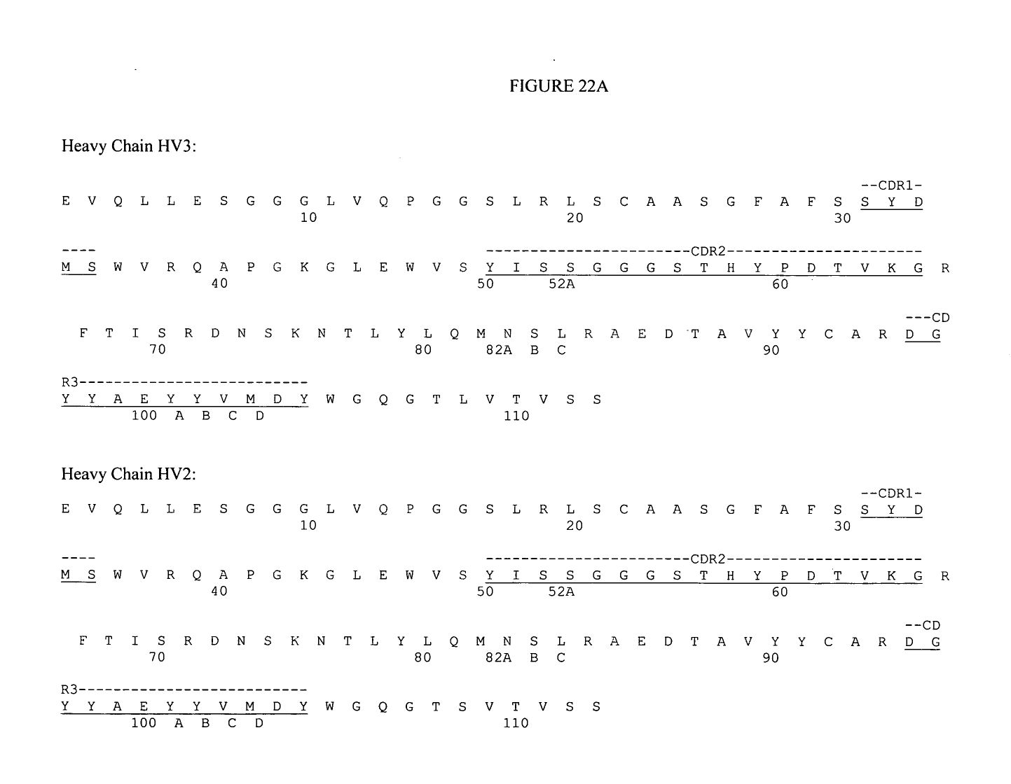

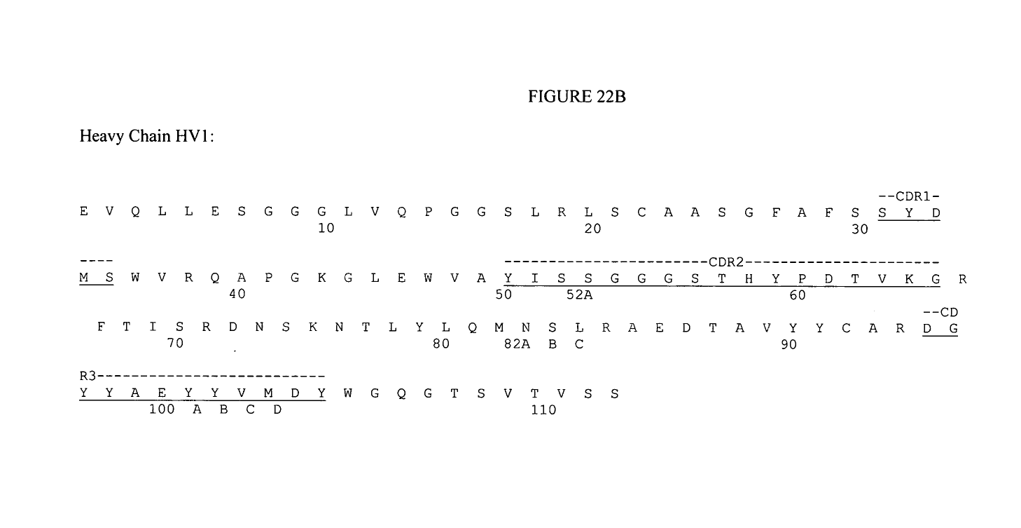

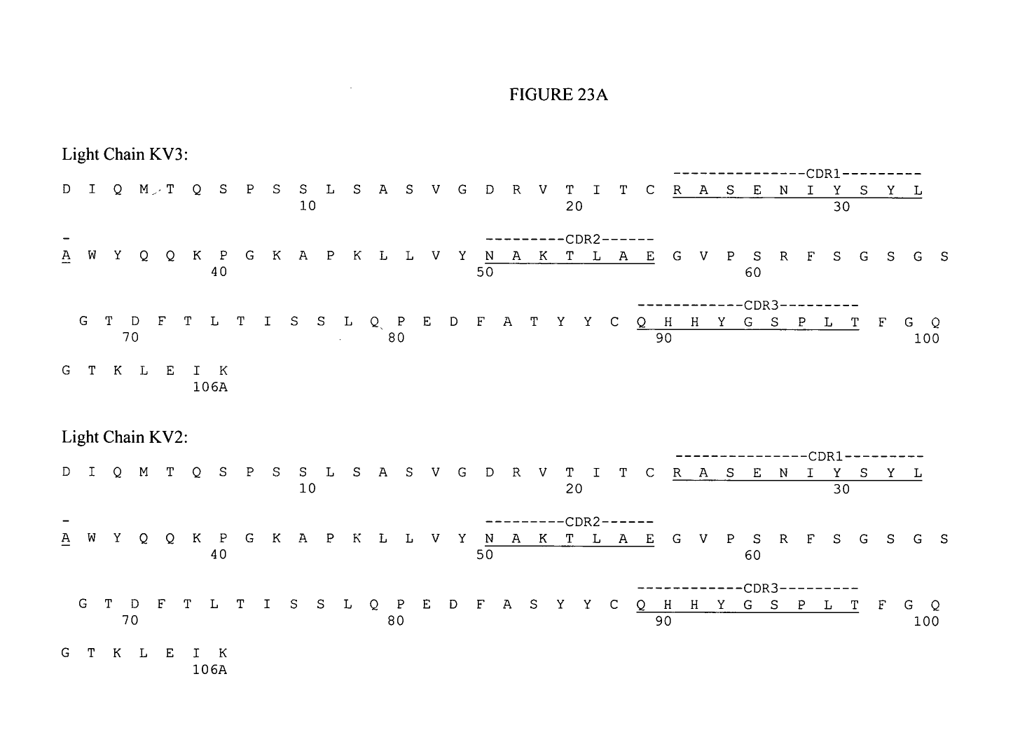

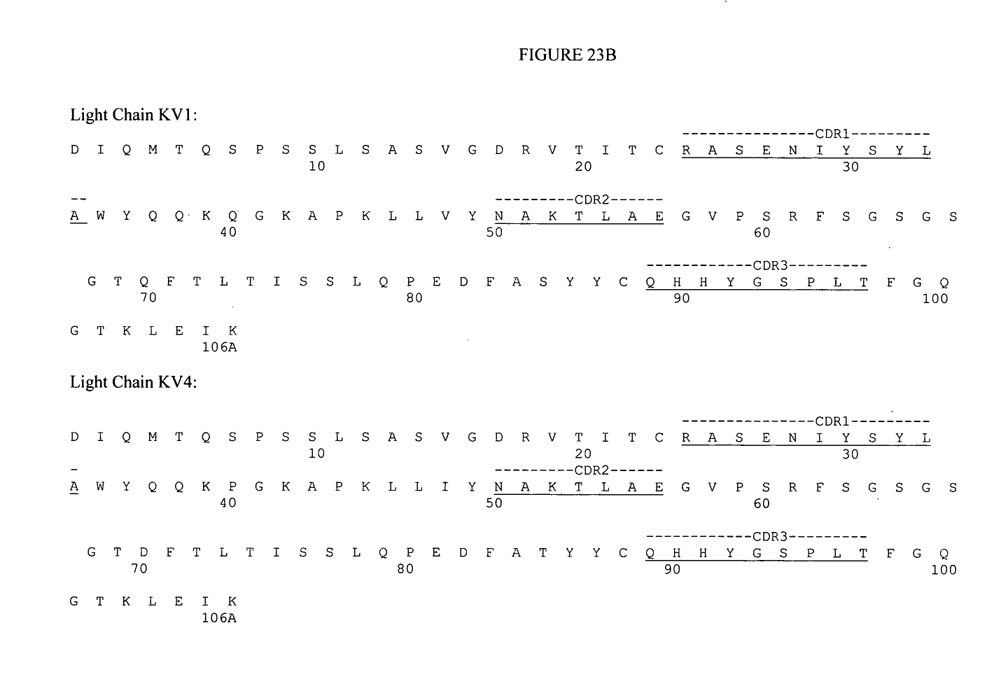

Humanized VH and VL sequences were designed by comparison of mouse AR36A36.11.1 sequences and homologous human VH and VL sequences. Sequences of the VH variants are given in FIGS. 22A and 22B and of the VL variants in FIGS. 23A and 23B. Humanized V region genes were constructed using the mouse AR36A36.11.1 VH and VL templates for PCR using long overlapping oligonucleotides to introduce amino acids from homologous human VH and VL sequences. Oligonucleotides used for generation of variant humanized VH and VL sequences are shown in FIGS. 19 and 20 respectively. Variant genes were cloned directly into the expression vectors pSVgpt and pSVhyg as detailed in US2004260069 (Hellendoom, Carr and Baker).

All combinations of variant humanized heavy and light chains (including the chimeric constructs) were transiently transfected into CHO-K1 cells (ECACC 85051005, Porton, UK) and supernatants harvested after 48 hours. The supernatants were quantified for antibody expression in IgG Fc/Kappa ELISA using purified human IgG1/Kappa (Sigma, Poole, UK) as standards. Immunosorb 96 well plates (Nalge nunc, Hereford, UK) were coated with mouse anti-human IgG Fc-specific antibody (16260 Sigma, Poole, UK) diluted at 1:1500 in 1×PBS (pH 7.4) at 37° C. for 1 hour. Plates were washed three times in PBS+0.05 percent Tween 20 before adding samples and standards, diluted in 2 percent BSA/PBS. Plates were incubated at room temperature for 1 hour before washing three times in PBS/Tween and adding 100 microliters/well of detecting antibody goat anti-human kappa light chain peroxidase conjugate (A7164 Sigma, Poole, UK) diluted 1:1000 in 2 percent BSA/PBS. Plates were incubated at room temperature for 1 hour before washing five times with PBS/Tween and bound antibody detected using OPD substrate (Sigma, Poole, UK). The assay was developed in the dark for 5 minutes before being stopped by the addition of 3 M HCl. The assay plate was then read in a MRX TCII plate reader (Dynex Technologies, Worthing, UK) at 490 nm.

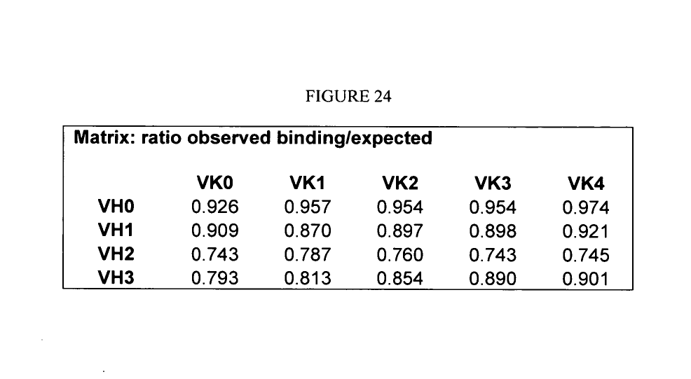

Binding of humanized variants was assayed in the competition binding ELISA described above. A standard curve was generated with varying concentrations (156.25 ng/mL to 5 micrograms/mL) of purified chimeric antibody ((ch)AR36A36.11.1) competing for binding with mouse AR36A36.11.1 to fixed MDA-MB-231 cells on a 96-well microtitre plate. Binding of mouse AR36A36.11.1 to MDA-MB-231 cells was detected with goat anti-mouse IgG:HRP conjugate (A2179 Sigma, Poole, UK) and developed using TMB substrate (Sigma, Poole, UK). Using the chimeric standard curve, the percentage inhibition expected at the concentrations tested was calculated for each variant and compared to that actually observed. The results were then normalized by dividing the observed inhibition of the test sample by the expected inhibition for each of the various heavy/light chain combinations. Thus a sample with an observed/expected ratio=1.0 has the same binding affinity as the chimeric antibody whereas a value >1.0 has reduced binding to CD59 and a sample with a ratio <1.0 has improved binding to CD59. The results are shown in FIG. 24.

Combinations of VH and VL genes were cloned into the dual vector pANT18 (pANT 18 vector is based on the plasmid pANT15 described previously, with the light chain cassette from pANT13 cloned into the SpeI/PciI restriction enzyme sites) and transfected into CHO/dhfr− cells (ECACC, 94060607) by electroporation and selected in media (high glucose DMEM with L-glutamine and Na pyruvate (Invitrogen, Paisley, UK) plus 5 percent dialysed FBS (Cat No. 26400-044 Invitrogen, Paisley, UK), Proline (Sigma, Poole, UK) and Penicllin/Streptomycin (Invitrogen, Paisley, UK)) depleted of Hypoxanthine and Thymidine. Antibodies were purified by Protein A affinity chromatography as above. The purified antibodies were filter sterilized before storing (in PBS pH 7.4) at +4° C. The concentrations of the antibodies were calculated by a human IgG1/kappa capture ELISA as above.

Three of the purified antibody samples were tested for binding to MDA-MB-231 cells expressing human CD59 via the competition ELISA as above. Varying concentrations of each antibody (156 ng/mL to 5 micrograms/mL) were mixed with purified mouse AR36A36.11.1 and added to microtiter plates coated with fixed MDA-MB-231 cells. Binding of mouse AR36A36.11.1 was detected with goat anti-mouse IgG (Fc):HRP conjugate as above. Absorbance 450 nm was measured on a plate reader and this was plotted against the test antibody concentration. The concentration of selected variants required to inhibit mouse AR36.A36.11.1 binding to MDA-MB-231 cells by 50 percent (IC₅₀) was calculated and compared to the chimeric antibody. The IC₅₀ for lead variant humanized antibodies and the chimeric were as follows;

(ch)AR36A36.11.1=26.27 micrograms/mL

(hu)AR36A36.11.1 variant HV3/KV3=11.71 micrograms/mL

(hu)AR36A36.11.1 variant HV2/KV3=11.68 micrograms/mL

(hu)AR36A36.11.1 variant HV2/KV4=13.30 micrograms/mL

### EXAMPLE 8

### Cell ELISA of Murine AR36A36.11.1, (ch)AR36A36.11.1 and Humanized Variants, (hu)AR36A36.11.1

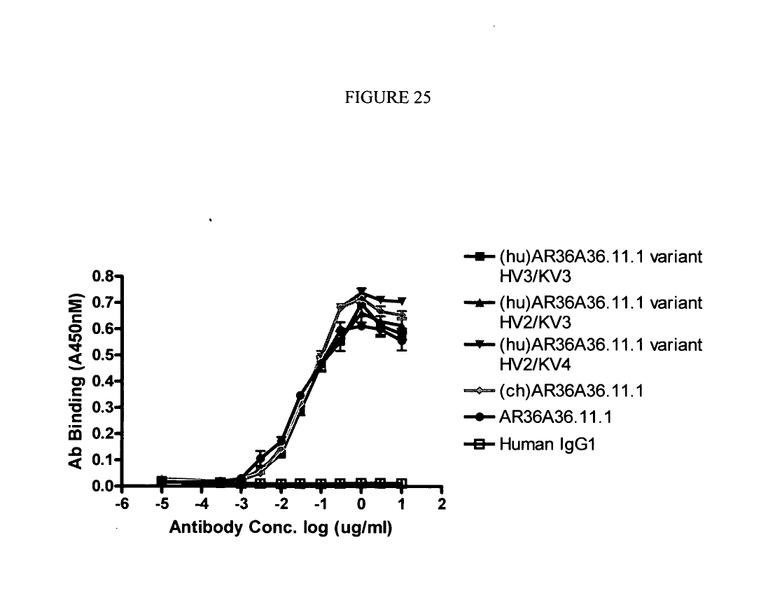

The three lead humanized variants, chimeric and murine AR36A36.11.1 along with isotype control were tested for binding to MDA-MB-231 cells expressing human CD59 via cell ELISA. The MDA-MB-231 cells were plated and fixed prior to use. The plates were washed thrice with PBS containing MgCl₂ and CaCl₂ at room temperature. 100 microliters of 2 percent paraformaldehyde diluted in PBS was added to each well for 10 minutes at room temperature and then discarded. The plates were again washed with PBS containing MgCl₂ and CaCl₂ three times at room temperature. Blocking was done with 100 microliters/well of 5 percent milk in wash buffer (PBS plus 0.05 percent Tween) for 1 hour at room temperature. The plates were washed thrice with wash buffer and varying concentrations of each antibody (0.3 ng/mL to 10 micrograms/mL) were added in 100 microliters/well of 1 percent milk in wash buffer (PBS plus 0.05 percent Tween) for 1 hour at room temperature. The plates were washed 3 times with wash buffer and 100 microliters/well of 1/10,000 dilution of goat anti-mouse IgG or goat anti-human IgG antibody conjugated to horseradish peroxidase (diluted in PBS containing 5 percent milk) was added. After 1 hour incubation at room temperature the plates were washed 3 times with wash buffer and 100 microliters/well of TMB substrate was incubated for 1-3 minutes at room temperature. The reaction was terminated with 100 microliters/well 2 M H₂S0₄ and the plate was read with Spectramax M5 (Molecular Devices) using Softmax Pro software at 450 nm with subtraction of absorbance at 595 nm. The antibody binding to MDA-MB-231 cells by 50 percent (EC₅₀) was calculated (FIG. 25). The EC₅₀ for the three variant humanized antibodies, chimeric and murine AR36A36.11.1 is as follows:

Murine AR36A36.11.1=0.091 micrograms/mL

(ch)AR36A36.11.1=0.561 micrograms/mL

(hu)AR36A36.11.1 variant HV3/KV3=0.096 micrograms/mL

(hu)AR36A36.11.1 variant HV2/KV3=0.092 micrograms/mL

(hu)AR36A36.11.1 variant HV2/KV4=0.055 micrograms/mL

### EXAMPLE 9

### Demonstration of In Vitro Complement-Dependent Cytotoxicity (CDC) Activity of the Murine and Humanized Variants of Antibody AR36A36.11.1

Therapeutic efficacy of murine AR36A36.11.1 has previously been demonstrated in xenograft tumor models of human breast cancer (as disclosed in Ser. No. 11/067,366 and in Examples 2 and 3 above). In order to elucidate possible mechanisms of action and to demonstrate in vitro efficacy of humanized clones of AR36A36.11.1, CDC activity was evaluated on the human breast cancer cell line MDA-MB-231. Established monolayers of MDA-MB-231 cells; two days post plating; were treated with both murine (20 micrograms/mL) and humanized (2, 0.2 and 0.02 micrograms/mL) antibody and allowed to bind for one hour (37° C.; 5 percent CO₂). Rabbit complement was added to yield a final concentration of 10 percent (v/v) and was allowed to incubate for an additional 3 hours at 37° C., 5 percent CO₂. CDC activity was evaluated by measuring the residual lactate dehydrogenase present in uncompromised cells using the Cytotox 96™ kit (Promega Corporation, Madison, Wis., USA). Each test antibody was evaluated in triplicate and the results were expressed as percent cytotoxicity, as compared to rabbit complement only treated wells, using the following equation: percent Cytotoxicity=100−[Test Antibody₍₄₉₂ₙₘ₎−Background₍₄₉₂ₙₘ₎]/Complement Only₍₄₉₂ₙₘ₎−Background₍₄₉₂ₙₘ₎]*100.

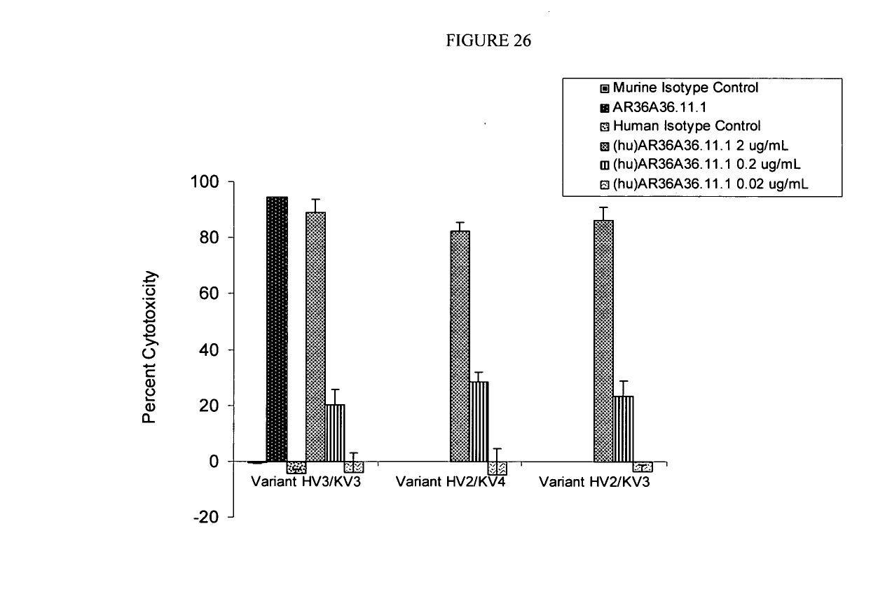

The results from this experiment (FIG. 26) demonstrate that the humanized variant clones of AR36A36.11.1 are capable of recruiting rabbit complement in a dose-dependent manner in MDA-MB-231 target cells. CDC activity was not observed in the breast cancer cells with isotype-matched control at the highest concentration (20 micrograms/mL). This data demonstrates that the complement dependent activity of murine AR36A36.11.1 is conserved during the humanization process.

### EXAMPLE 10

### Isolation of Competitive Binders

Given an antibody, an individual ordinarily skilled in the art can generate a competitively inhibiting CDMAB, for example a competing antibody, which is one that recognizes the same epitope (Belanger L et al. Clinica Chimica Acta 48:15-18 (1973)). One method entails immunizing with an immunogen that expresses the antigen recognized by the antibody. The sample may include but is not limited to tissues, isolated protein(s) or cell line(s). Resulting hybridomas could be screened using a competition assay, which is one that identifies antibodies that inhibit the binding of the test antibody, such as ELISA, FACS or Western blotting. Another method could make use of phage display antibody libraries and panning for antibodies that recognize at least one epitope of said antigen (Rubinstein J L et al. Anal Biochem 314:294-300 (2003)). In either case, antibodies are selected based on their ability to displace the binding of the original labeled antibody to at least one epitope of its target antigen. Such antibodies would therefore possess the characteristic of recognizing at least one epitope of the antigen as the original antibody.

### EXAMPLE 111

### Cloning of the Variable Regions of the AR36A36.11.1 Monoclonal Antibody

The sequences of the variable regions from the heavy (V_{H}) and light (V_{L}) chains of monoclonal antibody produced by the AR36A36.11.1 hybridoma cell line were determined (as disclosed in Example 7 above). To generate chimeric and humanized IgG, the variable light and variable heavy domains can be subcloned into an appropriate vector for expression (as disclosed in Example 7 above).

In another embodiment, AR36A36.11.1 or its de-immunized, chimeric or humanized version is produced by expressing a nucleic acid encoding the antibody in a transgenic animal, such that the antibody is expressed and can be recovered. For example, the antibody can be expressed in a tissue specific manner that facilitates recovery and purification. In one such embodiment, an antibody of the invention is expressed in the mammary gland for secretion during lactation. Transgenic animals include but are not limited to mice, goat and rabbit.

(i) Monoclonal Antibody

DNA encoding the monoclonal antibody (as disclosed Example 7 above) is readily isolated and sequenced using conventional procedures (e.g., by using oligonucleotide probes that are capable of binding specifically to genes encoding the heavy and light chains of the monoclonal antibodies). The hybridoma cell serves as a preferred source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences. Chimeric or hybrid antibodies also may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate.

(ii) Humanized Antibody

A humanized antibody has one or more amino acid residues introduced into it from a non-human source. These non-human amino acid residues are often referred to as “import” residues, which are typically taken from an “import” variable domain. Humanization can be performed using the method of Winter and co-workers by substituting rodent CDRs or CDR sequences for the corresponding sequences of a human antibody (Jones et al., Nature 321:522-525 (1986); Riechmann et al., Nature 332:323-327 (1988); Verhoeyen et al., Science 239:1534-1536 (1988); reviewed in Clark, Immunol. Today 21:397-402 (2000)).

A humanized antibody can be prepared by a process of analysis of the parental sequences and various conceptual humanized products using three-dimensional models of the parental and humanized sequences. Three dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e. the analysis of residues that influence the ability of the candidate immunoglobulin to bind its antigen. In this way, FR residues can be selected and combined from the consensus and import sequence so that the desired antibody characteristic, such as increased affinity for the target antigen(s), is achieved. In general, the CDR residues are directly and most substantially involved in influencing antigen binding.

(iii) Antibody Fragments

Various techniques have been developed for the production of antibody fragments. These fragments can be produced by recombinant host cells (reviewed in Hudson, Curr. Opin. Immunol. 11:548-557 (1999); Little et al., Immunol. Today 21:364-370 (2000)). For example, Fab′-SH fragments can be directly recovered from E. coli and chemically coupled to form F(ab′)₂ fragments (Carter et al., Biotechnology 10:163-167 (1992)). In another embodiment, the F(ab′)₂ is formed using the leucine zipper GCN4 to promote assembly of the F(ab′)₂ molecule. According to another approach, Fv, Fab or F(ab′)₂ fragments can be isolated directly from recombinant host cell culture.

### EXAMPLE 12

### A Composition Comprising the Antibody of the Present Invention

The antibody of the present invention can be used as a composition for preventing/treating cancer. The composition for preventing/treating cancer, which comprises the antibody of the present invention, can be administered as they are in the form of liquid preparations, or as pharmaceutical compositions of suitable preparations to human or mammals (e.g., rats, rabbits, sheep, swine, bovine, feline, canine, simian, etc.) orally or parenterally (e.g., intravascularly, intraperitoneally, subcutaneously, etc.). The antibody of the present invention may be administered in itself, or may be administered as an appropriate composition. The composition used for the administration may contain a pharmacologically acceptable carrier with the antibody of the present invention or its salt, a diluent or excipient. Such a composition is provided in the form of pharmaceutical preparations suitable for oral or parenteral administration.

Examples of the composition for parenteral administration are injectable preparations, suppositories, etc. The injectable preparations may include dosage forms such as intravenous, subcutaneous, intracutaneous and intramuscular injections, drip infusions, intraarticular injections, etc. These injectable preparations may be prepared by methods publicly known. For example, the injectable preparations may be prepared by dissolving, suspending or emulsifying the antibody of the present invention or its salt in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant (e.g., polysorbate 80, HCO-50 (polyoxyethylene (50 mols) adduct of hydrogenated castor oil)), etc. As the oily medium, there are employed, e.g., sesame oil, soybean oil, etc., which may be used in combination with a solubilizing agent such as benzyl benzoate, benzyl alcohol, etc. The injection thus prepared is usually filled in an appropriate ampoule. The suppository used for rectal administration may be prepared by blending the antibody of the present invention or its salt with conventional bases for suppositories. The composition for oral administration includes solid or liquid preparations, specifically, tablets (including dragees and film-coated tablets), pills, granules, powdery preparations, capsules (including soft capsules), syrup, emulsions, suspensions, etc. Such a composition is manufactured by publicly known methods and may contain a vehicle, a diluent or excipient conventionally used in the field of pharmaceutical preparations. Examples of the vehicle or excipient for tablets are lactose, starch, sucrose, magnesium stearate, etc.

Advantageously, the compositions for oral or parenteral use described above are prepared into pharmaceutical preparations with a unit dose suited to fit a dose of the active ingredients. Such unit dose preparations include, for example, tablets, pills, capsules, injections (ampoules), suppositories, etc. The amount of the aforesaid compound contained is generally 5 to 500 mg per dosage unit form; it is preferred that the antibody described above is contained in about 5 to about 100 mg especially in the form of injection, and in 10 to 250 mg for the other forms.

The dose of the aforesaid prophylactic/therapeutic agent or regulator comprising the antibody of the present invention may vary depending upon subject to be administered, target disease, conditions, route of administration, etc. For example, when used for the purpose of treating/preventing, e.g., breast cancer in an adult, it is advantageous to administer the antibody of the present invention intravenously in a dose of about 0.01 to about 20 mg/kg body weight, preferably about 0.1 to about 10 mg/kg body weight and more preferably about 0.1 to about 5 mg/kg body weight, about 1 to 5 times/day, preferably about 1 to 3 times/day. In other parenteral and oral administration, the agent can be administered in a dose corresponding to the dose given above. When the condition is especially severe, the dose may be increased according to the condition.

The antibody of the present invention may be administered as it stands or in the form of an appropriate composition. The composition used for the administration may contain a pharmacologically acceptable carrier with the aforesaid antibody or its salts, a diluent or excipient. Such a composition is provided in the form of pharmaceutical preparations suitable for oral or parenteral administration (e.g., intravascular injection, subcutaneous injection, etc.). Each composition described above may further contain other active ingredients. Furthermore, the antibody of the present invention may be used in combination with other drugs, for example, alkylating agents (e.g., cyclophosphamide, ifosfamide, etc.), metabolic antagonists (e.g., methotrexate, 5-fluorouracil, etc.), anti-tumor antibiotics (e.g., mitomycin, adriamycin, etc.), plant-derived anti-tumor agents (e.g., vincristine, vindesine, Taxol, etc.), cisplatin, carboplatin, etoposide, irinotecan, etc. The antibody of the present invention and the drugs described above may be administered simultaneously or at staggered times to the patient.

The method of treatment described herein, particularly for cancers, may also be carried out with administration of other antibodies or chemotherapeutic agents. For example, an antibody against EGFR, such as ERBITUX® (cetuximab), may also be administered, particularly when treating colon cancer. ERBITUX® has also been shown to be effective for treatment of psoriasis. Other antibodies for combination use include Herceptin® (trastuzumab) particularly when treating breast cancer, AVASTIN® particularly when treating colon cancer and SGN-15 particularly when treating non-small cell lung cancer. The administration of the antibody of the present invention with other antibodies/chemotherapeutic agents may occur simultaneously, or separately, via the same or different route.

The chemotherapeutic agent/other antibody regimens utilized include any regimen believed to be optimally suitable for the treatment of the patient's condition. Different malignancies can require use of specific anti-tumor antibodies and specific chemotherapeutic agents, which will be determined on a patient to patient basis. In a preferred embodiment of the invention, chemotherapy is administered concurrently with or, more preferably, subsequent to antibody therapy. It should be emphasized, however, that the present invention is not limited to any particular method or route of administration.

The preponderance of evidence shows that AR36A36.11.1 mediates anti-cancer effects and prolongs survival through ligation of epitopes present on CD59. It has previously been shown, as disclosed in Ser. No. 11/361,153, that the AR36A36.11.1 antibody can be used to immunoprecipitate the cognate antigen from expressing cells such as MDA-MB-231 cells. Further it could be shown that AR36A36.11.1, chimeric AR36A36.11.1 or humanized variants, (hu)AR36A36.11.1 can be used in the detection of cells and/or tissues which express a CD59 antigenic moiety which specifically binds thereto, utilizing techniques illustrated by, but not limited to FACS, cell ELISA or IHC. As with the AR36A36.11.1 antibody, other anti-CD59 antibodies could be used to immunoprecipitate and isolate other forms of the CD59 antigen, and the antigen can also be used to inhibit the binding of those antibodies to the cells or tissues that express the antigen using the same types of assays.

SEQ IDs

| | |
|---|---|
| SEQ | |
| ID | Sequence |

| | | |
|---|---|---|
| Heavy CDR1 | 1 | SYDMS |
| Heavy CDR2 | 2 | YISSGGGSTHYPDTVKG |
| Heavy CDR3 | 3 | DGYYAEYYVMDY |
| Light CDR1 | 4 | RASENIYSYLA |
| Light CDR2 | 5 | NAKTLAE |
| Light CDR3 | 6 | QHHYGSPLT |
| HV3 | 7 | EVQLLESGGGLVQPGGSLRLSCAASGFAFSSYDMSW |
| | | VRQAPGKGLEWVSYISSGGGSTHYPDTVKGRFTISR |
| | | DNSKNTLYLQMNSLRAEDTAVYYCARDGYYAEYYVM |
| | | DYWGQGTLVTVSS |
| KV3 | 8 | DIQMTQSPSSLSASVGDRVTITCRASENIYSYLAWY |
| | | QQKPGKAPKLLVYNAKTLAEGVPSRFSGSGSGTDFT |
| | | LTISSLQPEDFATYYCQHHYGSPLTFGQGTKLEIK |
| HV2 | 9 | E V Q L L E S G G G L V Q P G G S L |
| | | R L S C A A S G F A F S S Y D M S W |
| | | V R Q A P G K G L E W V S Y I S S G |
| | | G G S T H Y P D T V K G R F T I S R |
| | | D N S K N T L Y L Q M N S L R A E D |
| | | T A V Y Y C A R D G Y Y A E Y Y V M |
| | | D Y W G Q G T S V T V S S |
| KV4 | 10 | D I Q M T Q S P S S L S A S V G D R |
| | | V T I T C R A S E N I Y S Y L A W Y |
| | | Q Q K P G K A P K L L I Y N A K T L |
| | | A E G V P S R F S G S G S G T D F T |
| | | L T I S S L Q P E D F A T Y Y C Q H |
| | | H Y G S P L T F G Q G T K L E I K |

All patents and publications mentioned in this specification are indicative of the levels of those skilled in the art to which the invention pertains. All patents and publications are herein incorporated by reference to the same extent as if each individual publication was specifically and individually-indicated to be incorporated by reference.

It is to be understood that while a certain form of the invention is illustrated, it is not to be limited to the specific form or arrangement of parts herein described and shown. It will be apparent to those skilled in the art that various changes may be made without departing from the scope of the invention and the invention is not to be considered limited to what is shown and described in the specification. One skilled in the art will readily appreciate that the present invention is well adapted to carry out the objects and obtain the ends and advantages mentioned, as well as those inherent therein. Any oligonucleotides, peptides, polypeptides, biologically related compounds, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

1. A method of reduction of a human breast, prostate, lung or colon tumor in a mammal, wherein said human breast, prostate, lung or colon tumor expresses at least one epitope of an antigen which specifically binds to the isolated monoclonal antibody produced by the hybridoma cell line deposited with the IDAC as accession number 280104-02 or a CDMAB thereof, which CDMAB is characterized by an ability to competitively inhibit binding of said isolated monoclonal antibody to its target antigen, comprising administering to said mammal said isolated monoclonal antibody or CDMAB thereof in an amount effective to result in a reduction of said mammal's breast, prostate, lung or colon tumor burden.

2. The method of claim 1 wherein said isolated monoclonal antibody is conjugated to a cytotoxic moiety.

3. The method of claim 2 wherein said cytotoxic moiety is a radioactive isotope.

4. The method of claim 1 wherein said isolated monoclonal antibody or CDMAB thereof activates complement.

5. The method of claim 1 wherein said isolated monoclonal antibody or CDMAB thereof mediates antibody dependent cellular cytotoxicity.

6. The method of claim 1 wherein said isolated monoclonal antibody is a humanized antibody of the isolated monoclonal antibody produced by the hybridoma deposited with the IDAC as accession number 280104-02 or an antigen binding fragment produced from said humanized antibody.

7. The method of claim 1 wherein said isolated monoclonal antibody is a chimeric antibody of the isolated monoclonal antibody produced by the hybridoma deposited with the IDAC as accession number 280104-02 or an antigen binding fragment produced from said chimeric antibody.

8. A method of reduction of a human breast, prostate, lung or colon tumor susceptible to antibody induced cellular cytotoxicity in a mammal, wherein said human breast, prostate, lung or colon tumor expresses at least one epitope of an antigen which specifically binds to the isolated monoclonal antibody produced by the hybridoma cell line deposited with the IDAC as accession number 280104-02 or a CDMAB thereof, which CDMAB is characterized by an ability to competitively inhibit binding of said isolated monoclonal antibody to its target antigen, comprising administering to said mammal said isolated monoclonal antibody or said CDMAB thereof in an amount effective to result in a reduction of said mammal's breast, prostate, lung or colon tumor burden.

9. The method of claim 8 wherein said isolated monoclonal antibody is conjugated to a cytotoxic moiety.

10. The method of claim 9 wherein said cytotoxic moiety is a radioactive isotope.

11. The method of claim 8 wherein said isolated monoclonal antibody or CDMAB thereof activates complement.

12. The method of claim 8 wherein said isolated monoclonal antibody or CDMAB thereof mediates antibody dependent cellular cytotoxicity.

13. The method of claim 8 wherein said isolated monoclonal antibody is a humanized antibody of the isolated monoclonal antibody produced by the hybridoma deposited with the IDAC as accession number 280104-02 or an antigen binding fragment produced from said humanized antibody.

14. The method of claim 8 wherein said isolated monoclonal antibody is a chimeric antibody of the isolated monoclonal antibody produced by the hybridoma deposited with the IDAC as accession number 280104-02 or an antigen binding fragment produced from said chimeric antibody.

15. A method of reduction of a human breast, prostate, lung or colon tumor in a mammal, wherein said human breast, prostate, lung or colon tumor expresses at least one epitope of an antigen which specifically binds to the isolated monoclonal antibody produced by the hybridoma deposited with the IDAC as accession number 280104-02 or a CDMAB thereof, which CDMAB is characterized by an ability to competitively inhibit binding of said isolated monoclonal antibody to its target antigen, comprising administering to said mammal said monoclonal antibody or CDMAB thereof in conjunction with at least one chemotherapuetic agent in an amount effective to result in a reduction of said mammal's breast, prostate, lung or colon tumor burden.

16. The method of claim 15 wherein said isolated monoclonal antibody is conjugated to a cytotoxic moiety.

17. The method of claim 16 wherein said cytotoxic moiety is a radioactive isotope.

18. The method of claim 15 wherein said isolated monoclonal antibody or CDMAB thereof activates complement.

19. The method of claim 15 wherein said isolated monoclonal antibody or CDMAB thereof mediates antibody dependent cellular cytotoxicity.

20. The method of claim 15 wherein said isolated monoclonal antibody is a humanized antibody of the isolated monoclonal antibody produced by the hybridoma deposited with the IDAC as accession number 280104-02 or an antigen binding fragment produced from said humanized antibody.

21. The method of claim 15 wherein said isolated monoclonal antibody is a chimeric antibody of the isolated monoclonal antibody produced by the hybridoma deposited with the IDAC as accession number 280104-02 or an antigen binding fragment produced from said chimeric antibody.

22. Use of monoclonal antibodies for reduction of human breast, pancreatic, ovarian, prostate or colon tumor burden, wherein said human breast, pancreatic, ovarian, prostate or colon tumor expresses at least one epitope of an antigen which specifically binds to the isolated monoclonal antibody produced by the hybridoma deposited with the IDAC as accession number 280104-02 or a CDMAB thereof, which CDMAB is characterized by an ability to competitively inhibit binding of said isolated monoclonal antibody to its target antigen, comprising administering to said mammal said monoclonal antibody or CDMAB thereof in an amount effective to result in a reduction of said mammal's human breast, pancreatic, ovarian, prostate or colon tumor burden.

23. The method of claim 22 wherein said isolated monoclonal antibody is conjugated to a cytotoxic moiety.

24. The method of claim 23 wherein said cytotoxic moiety is a radioactive isotope.

25. The method of claim 22 wherein said isolated monoclonal antibody or CDMAB thereof activates complement.

26. The method of claim 22 wherein said isolated monoclonal antibody or CDMAB thereof mediates antibody dependent cellular cytotoxicity.

27. The method of claim 22 wherein said isolated monoclonal antibody is a humanized antibody of the isolated monoclonal antibody produced by the hybridoma deposited with the IDAC as accession number 280104-02.

28. The method of claim 22 wherein said isolated monoclonal antibody is a chimeric antibody of the isolated monoclonal antibody produced by the hybridoma deposited with the IDAC as accession number 280104-02.

29. Use of monoclonal antibodies for reduction of human breast, pancreatic, ovarian, prostate or colon tumor burden, wherein said human breast, pancreatic, ovarian, prostate or colon tumor expresses at least one epitope of an antigen which specifically binds to the isolated monoclonal antibody produced by the hybridoma deposited with the IDAC as accession number 280104-02 or a CDMAB thereof, which CDMAB is characterized by an ability to competitively inhibit binding of said isolated monoclonal antibody to its target antigen, comprising administering to said mammal said monoclonal antibody or CDMAB thereof, in conjunction with at least one chemotherapeutic agent in an amount effective to result in a reduction of said mammal's human breast, pancreatic, ovarian, prostate or colon tumor burden.

30. The method of claim 29 wherein said isolated monoclonal antibody is conjugated to a cytotoxic moiety.

31. The method of claim 30 wherein said cytotoxic moiety is a radioactive isotope.

32. The method of claim 29 wherein said isolated monoclonal antibody or CDMAB thereof activates complement.

33. The method of claim 29 wherein said isolated monoclonal antibody or CDMAB thereof mediates antibody dependent cellular cytotoxicity.

34. The method of claim 29 wherein said isolated monoclonal antibody is a humanized antibody of the isolated monoclonal antibody produced by the hybridoma deposited with the IDAC as accession number 280104-02.

35. The method of claim 29 wherein said isolated monoclonal antibody is a chimeric antibody of the isolated monoclonal antibody produced by the hybridoma deposited with the IDAC as accession number 280104-02.

36. A process for reduction of a human breast, pancreatic, ovarian, prostate or colon tumor which expresses at least one epitope of human CD59 antigen which is specifically bound by the isolated monoclonal antibody produced by hybridoma cell line AR36A36.11.1 having IDAC Accession No. 280104-02, comprising:
administering to an individual suffering from said human tumor, at least one isolated monoclonal antibody or CDMAB thereof that binds the same epitope or epitopes as those bound by the isolated monoclonal antibody produced by the hybridoma cell line AR36A36.11.1 having IDAC Accession No. 280104-02;
wherein binding of said epitope or epitopes results in a reduction of breast, pancreatic, ovarian, prostate or colon tumor burden.

37. A process for reduction of a human breast, pancreatic, ovarian, prostate or colon tumor which expresses at least one epitope of human CD59 antigen which is specifically bound by the isolated monoclonal antibody produced by hybridoma cell line AR36A36.11.1 having IDAC Accession No. 280104-02, comprising:
administering to an individual suffering from said human tumor, at least one isolated monoclonal antibody or CDMAB thereof, that binds the same epitope or epitopes as those bound by the isolated monoclonal antibody produced by the hybridoma cell line AR36A36.11.1 having IDAC Accession No. 280104-02; in conjunction with at least one chemotherapeutic agent;
wherein said administration results in a reduction of tumor burden.

38. A method of extending survival and delaying disease progression by treating a human breast, pancreatic, ovarian, prostate or colon tumor in a mammal, wherein said tumor expresses an antigen which specifically binds to the isolated monoclonal antibody produced by the hybridoma cell line AR36A36.11.1 having IDAC Accession No. 280104-02, or an antigen binding fragment produced from said isolated monoclonal antibody comprising administering to said mammal said monoclonal antibody in an amount effective to reduce said mammal's tumor burden, whereby disease progression is delayed and survival is extended.

39. A method of extending survival and delaying disease progression by treating a human breast, pancreatic, ovarian, prostate or colon tumor in a mammal, wherein said tumor expresses CD59 which specifically binds to the isolated monoclonal antibody produced by the hybridoma cell line AR36A36.11.1 having IDAC Accession No. 280104-02, or a CD59 binding fragment produced from said isolated monoclonal antibody comprising administering to said mammal said monoclonal antibody in an amount effective to reduce said mammal's tumor burden, whereby disease progression is delayed and survival is extended.

40. A method for inducing complement dependent cytotoxicity of cancerous cells, which express at least one epitope of CD59 on the cell's surface, which at least one epitope, when bound by the isolated monoclonal antibody produced by the hybridoma deposited with the IDAC as 280104-02 or an antigen binding fragment produced from said isolated monoclonal antibody results in cell cytotoxicity, comprising:
providing the isolated monoclonal antibody produced by the hybridoma deposited with the IDAC as 280104-02 or an antigen binding fragment produced from said isolated monoclonal antibody, and
contacting said cancerous cells with said isolated monoclonal antibody or said antigen binding fragment;
whereby cytotoxicity occurs as a result of binding of said isolated monoclonal antibody or said antigen binding fragment with said at least one epitope of TROP-2.

41. The method of claim 40 wherein said isolated monoclonal antibody is conjugated to a cytotoxic moiety.

42. The method of claim 41 wherein said cytotoxic moiety is a radioactive isotope.

43. The method of claim 40 wherein said isolated monoclonal antibody activates complement.

44. The method of claim 40 wherein said isolated monoclonal antibody mediates cellular cytotoxicity.

45. The method of claim 40 wherein said monoclonal antibody is a humanized antibody of the isolated monoclonal antibody produced by the hybridoma deposited with the IDAC as 280104-02 or an antigen binding fragment produced from said humanized antibody.

46. The method of claim 40 wherein said monoclonal antibody is a chimeric antibody of the isolated monoclonal antibody produced by the hybridoma deposited with the IDAC as 280104-02 or an antigen binding fragment produced from said chimeric antibody.

47. A method for inducing complement dependent cytotoxicity of cancerous cells, which express at least one epitope of CD59 on the cell's surface, which at least one epitope, when bound by the isolated monoclonal antibody produced by the hybridoma deposited with the IDAC as 280104-02 or an antigen binding fragment produced from said isolated monoclonal antibody results in cell cytotoxicity, comprising:
providing an isolated monoclonal antibody which competitively inhibits binding of the isolated monoclonal antibody produced by the hybridoma deposited with the IDAC as 280104-02 or of an antigen binding fragment produced from said isolated monoclonal antibody, and which when bound by said at least one epitope of CD59, results in cell cytotoxicity; and
contacting said cancerous cells with said isolated monoclonal antibody or said antigen binding fragment;
whereby cytotoxicity occurs as a result of binding of said isolated monoclonal antibody or said antigen binding fragment with said at least one epitope of CD59.

48. A monoclonal antibody which specifically binds to the same epitope or epitopes as the isolated monoclonal antibody produced by the hybridoma deposited with the IDAC as accession number 280104-02.

49. An isolated monoclonal antibody or CDMAB thereof, which specifically binds to human CD59, in which the isolated monoclonal antibody or CDMAB thereof reacts with the same epitope or epitopes of human CD59 as the isolated monoclonal antibody produced by a hybridoma cell line AR36A36.11.1 having IDAC Accession No. 280104-02; said isolated monoclonal antibody or CDMAB thereof being characterized by an ability to competitively inhibit binding of said isolated monoclonal antibody to its target human CD59 antigen.

50. An isolated monoclonal antibody or CDMAB thereof that recognizes the same epitope or epitopes as those recognized by the isolated monoclonal antibody produced by the hybridoma cell line AR36A36.11.1 having IDAC Accession No. 280104-02; said monoclonal antibody or CDMAB thereof being characterized by an ability to competitively inhibit binding of said isolated monoclonal antibody to its target epitope or epitopes.

51. A monoclonal antibody that specifically binds the same epitope or epitopes of human CD59 as the isolated monoclonal antibody produced by the hybridoma cell line AR36A36.11.1 having IDAC Accession No. 280104-02, comprising:
a heavy chain variable region comprising the complementarity determining region amino acid sequences of SEQ ID NO:1, SEQ ID NO:2, and SEQ ID NO:3; and a light chain variable region comprising the complementarity determining region amino acid sequences of SEQ ID NO:4, SEQ ID NO:5, or SEQ ID NO:6;
or a human CD59 binding fragment thereof.

52. A monoclonal antibody that specifically binds the same epitope or epitopes of human CD59 as the isolated monoclonal antibody produced by the hybridoma cell line AR36A36.11.1 having IDAC Accession No. 280104-02, comprising:
a heavy chain variable region comprising the complementarity determining region amino acid sequences of SEQ ID NO:1, SEQ ID NO:2, and SEQ ID NO:3; and a light chain variable region comprising the complementarity determining region amino acid sequences of SEQ ID NO:4, SEQ ID NO:5, or SEQ ID NO:6; and variable domain framework regions from the heavy and light chains of a human antibody or human antibody consensus framework;
or a human CD59 binding fragment thereof.

53. A monoclonal antibody that specifically binds human CD59, wherein said monoclonal antibody comprises a heavy chain variable region amino acid sequence of SEQ ID NO:7; and a light chain variable region amino acid sequence selected of SEQ ID NO:8;
or a human CD59 binding fragment thereof.

54. A humanized antibody that specifically binds the same epitope or epitopes of human CD59 as the isolated monoclonal antibody produced by the hybridoma cell line AR36A36.11.1 having IDAC Accession No. 280104-02, comprising:
a heavy chain variable region comprising the complementarity determining region amino acid sequences of SEQ ID NO:1, SEQ ID NO:2, and SEQ ID NO:3; and a light chain variable region comprising the complementarity determining region amino acid sequences of SEQ ID NO:4, SEQ ID NO:5, or SEQ ID NO:6;
or a human CD59 binding fragment thereof.

55. A humanized antibody that specifically binds the same epitope or epitopes of human CD59 as the isolated monoclonal antibody produced by the hybridoma cell line AR36A36.11.1 having IDAC Accession No. 280104-02, comprising:
a heavy chain variable region comprising the complementarity determining region amino acid sequences of SEQ ID NO:1, SEQ ID NO:2, and SEQ ID NO:3; and a light chain variable region comprising the complementarity determining region amino acid sequences of SEQ ID NO:4, SEQ ID NO:5, or SEQ ID NO:6; and variable domain framework regions from the heavy and light chains of a human antibody or human antibody consensus framework;
or a human CD59 binding fragment thereof.

56. A humanized antibody that specifically binds human CD59, wherein said monoclonal antibody comprises a heavy chain variable region amino acid sequence of SEQ ID NO:7; and a light chain variable region amino acid sequence selected of SEQ ID NO:8;
or a human CD59 binding fragment thereof.

57. A humanized antibody that specifically binds human CD59, wherein said monoclonal antibody comprises a heavy chain variable region amino acid sequence of SEQ ID NO:9; and a light chain variable region amino acid sequence selected of SEQ ID NO:8;
or a human CD59 binding fragment thereof.

58. A humanized antibody that specifically binds human CD59, wherein said monoclonal antibody comprises a heavy chain variable region amino acid sequence of SEQ ID NO:9; and a light chain variable region amino acid sequence selected of SEQ ID NO:10;
or a human CD59 binding fragment thereof.

59. A composition effective for treating a human pancreatic, prostate, ovarian, breast or colon tumor comprising in combination:
an antibody or CDMAB of any one of claims 1, 2, 3, 6, 7, 8, 17, 49, 50, 54, 55, 56, 57 or 58;
a conjugate of said antibody or an antigen binding fragment thereof with a member selected from the group consisting of cytotoxic moieties, enzymes, radioactive compounds, cytokines, interferons, target or reporter moieties and hematogenous cells; and
a requisite amount of a pharmacologically acceptable carrier;
wherein said composition is effective for treating said human breast, prostate, lung or colon tumor.

60. A composition effective for treating a human breast, prostate, lung or colon tumor comprising in combination:
an antibody or CDMAB of any one of claims 1, 2, 3, 6, 7, 8, 17, 49, 50, 54, 55, 56, 57 or 58; and a requisite amount of a pharmacologically acceptable carrier;
wherein said composition is effective for treating said human breast, prostate, lung or colon tumor.

61. A composition effective for treating a human breast, prostate, lung or colon tumor comprising in combination:
a conjugate of an antibody, antigen binding fragment, or CDMAB of any one of claims 1, 2, 3, 6, 7, 8, 17, 49, 50, 54, 55, 56, 57 or 58; with a member selected from the group consisting of cytotoxic moieties, enzymes, radioactive compounds, cytokines, interferons, target or reporter moieties and hematogenous cells; and
a requisite amount of a pharmacologically acceptable carrier;
wherein said composition is effective for treating said human breast, prostate, lung or colon tumor.

62. An assay kit for detecting the presence of a human cancerous tumor, wherein said human cancerous tumor expresses at least one epitope of an antigen which specifically binds to the isolated monoclonal antibody produced by the hybridoma deposited with the IDAC as accession number 280104-02 or a CDMAB thereof, which CDMAB is characterized by an ability to competitively inhibit binding of said isolated monoclonal antibody to its target antigen, the kit comprising the isolated monoclonal antibody produced by the hybridoma deposited with the IDAC as accession number 280104-02 or a CDMAB thereof, and means for detecting whether the monoclonal antibody, or a CDMAB thereof, is bound to a polypeptide whose presence, at a particular cut-off level, is diagnostic of said presence of said human cancerous tumor.


SEQUENCE LISTING (111 sequences)

SEQ ID NO: 1 - protein, 5 aa, Artificial Sequence
  Description of Artificial Sequence  Synthetic peptide
SYDMS

SEQ ID NO: 2 - protein, 17 aa, Artificial Sequence
  Description of Artificial Sequence  Synthetic peptide
YISSGGGSTHYPDTVKG

SEQ ID NO: 3 - protein, 12 aa, Artificial Sequence
  Description of Artificial Sequence  Synthetic peptide
DGYYAEYYVMDY

SEQ ID NO: 4 - protein, 11 aa, Artificial Sequence
  Description of Artificial Sequence  Synthetic peptide
RASENIYSYLA

SEQ ID NO: 5 - protein, 7 aa, Artificial Sequence
  Description of Artificial Sequence  Synthetic peptide
NAKTLAE

SEQ ID NO: 6 - protein, 9 aa, Artificial Sequence
  Description of Artificial Sequence  Synthetic peptide
QHHYGSPLT

SEQ ID NO: 7 - protein, 121 aa, Artificial Sequence
  Description of Artificial Sequence  Synthetic polypeptide
EVQLLESGGGLVQPGGSLRLSCAASGFAFSSYDMSWVRQAPGKGLEWVSYISSGGGSTHYPDTVKGRFTISRDNSKNTLYLQMNSLRAEDTAVYYCARDGYYAEYYVMDYWGQGTLVTVSS

SEQ ID NO: 8 - protein, 107 aa, Artificial Sequence
  Description of Artificial Sequence  Synthetic polypeptide
DIQMTQSPSSLSASVGDRVTITCRASENIYSYLAWYQQKPGKAPKLLVYNAKTLAEGVPSRFSGSGSGTDFTLTISSLQPEDFATYYCQHHYGSPLTFGQGTKLEIK

SEQ ID NO: 9 - protein, 121 aa, Artificial Sequence
  Description of Artificial Sequence  Synthetic polypeptide
EVQLLESGGGLVQPGGSLRLSCAASGFAFSSYDMSWVRQAPGKGLEWVSYISSGGGSTHYPDTVKGRFTISRDNSKNTLYLQMNSLRAEDTAVYYCARDGYYAEYYVMDYWGQGTSVTVSS

SEQ ID NO: 10 - protein, 107 aa, Artificial Sequence
  Description of Artificial Sequence  Synthetic polypeptide
DIQMTQSPSSLSASVGDRVTITCRASENIYSYLAWYQQKPGKAPKLLIYNAKTLAEGVPSRFSGSGSGTDFTLTISSLQPEDFATYYCQHHYGSPLTFGQGTKLEIK

SEQ ID NO: 11 - protein, 6 aa, Artificial Sequence
  Description of Artificial Sequence  Synthetic peptide
VYNKCW

SEQ ID NO: 12 - protein, 6 aa, Artificial Sequence
  Description of Artificial Sequence  Synthetic peptide
NFNDVT

SEQ ID NO: 13 - protein, 4 aa, Artificial Sequence
  Description of Artificial Sequence  Synthetic peptide
LTYY

SEQ ID NO: 14 - protein, 28 aa, Artificial Sequence
  Description of Artificial Sequence  Synthetic peptide
VYNKCWKFEHCNFNDVTTRLRENELTYY

SEQ ID NO: 15 - protein, 14 aa, Artificial Sequence [sequence omitted]

SEQ ID NO: 16 - protein, 14 aa, Artificial Sequence
  Description of Artificial Sequence  Synthetic peptide
CLTYYACVYNKAWC

SEQ ID NO: 17 - protein, 14 aa, Artificial Sequence
  Description of Artificial Sequence  Synthetic peptide
CLANFNCVYNKAWC

SEQ ID NO: 18 - protein, 14 aa, Artificial Sequence
  Description of Artificial Sequence  Synthetic peptide
CVYNKAWCLANFNC

SEQ ID NO: 19 - protein, 16 aa, Artificial Sequence
  Description of Artificial Sequence  Synthetic peptide
CFNDVTTRCVYNKAWC

SEQ ID NO: 20 - protein, 29 aa, Artificial Sequence
  Description of Artificial Sequence  Synthetic peptide
CFEHANFNDVTTRLCKAGLQVYNKAWKFC

SEQ ID NO: 21 - protein, 29 aa, Artificial Sequence
  Description of Artificial Sequence  Synthetic peptide
CHANFNDVTTRLRECKAGLQVYNKAWKFC

SEQ ID NO: 22 - protein, 14 aa, Artificial Sequence
  Description of Artificial Sequence  Synthetic peptide
LITCAGLQVYCKAW

SEQ ID NO: 23 - protein, 14 aa, Artificial Sequence
  Description of Artificial Sequence  Synthetic peptide
CALITKCVYNKAWC

SEQ ID NO: 24 - protein, 13 aa, Artificial Sequence
  Description of Artificial Sequence  Synthetic peptide
CLANFNCALITKC

SEQ ID NO: 25 - protein, 28 aa, Artificial Sequence
  Description of Artificial Sequence  Synthetic peptide
CKTAVNCGSGCALITKCGSGCVYNKAWC

SEQ ID NO: 26 - protein, 13 aa, Artificial Sequence
  Description of Artificial Sequence  Synthetic peptide
CALITKCLTYYAC

SEQ ID NO: 27 - protein, 29 aa, Artificial Sequence
  Description of Artificial Sequence  Synthetic peptide
CYNKAWKFEHANFNCKAGLQVYNKAWKFC

SEQ ID NO: 28 - protein, 13 aa, Artificial Sequence
  Description of Artificial Sequence  Synthetic peptide
CLTYYACALITKC

SEQ ID NO: 29 - protein, 30 aa, Artificial Sequence
  Description of Artificial Sequence  Synthetic peptide
CALITKCGSGCFNDVTTRCGSGCVYNKAWC

SEQ ID NO: 30 - protein, 30 aa, Artificial Sequence
  Description of Artificial Sequence  Synthetic peptide
CFNDVTTRCGSGCVYNKAWCGSGCLTYYAC

SEQ ID NO: 31 - protein, 28 aa, Artificial Sequence
  Description of Artificial Sequence  Synthetic peptide
CVYNKAWCGSGCLANFNCGSGCLTYYAC

SEQ ID NO: 32 - protein, 30 aa, Artificial Sequence
  Description of Artificial Sequence  Synthetic peptide
CLTYYACGSGCFNDVTTRCGSGCVYNKAWC

SEQ ID NO: 33 - protein, 28 aa, Artificial Sequence
  Description of Artificial Sequence  Synthetic peptide
CALITKCGSGCLANFNCGSGCVYNKAWC

SEQ ID NO: 34 - protein, 32 aa, Artificial Sequence
  Description of Artificial Sequence  Synthetic peptide
CFNDVTTRCGSGCQAYNAPNCGSGCVYNKAWC

SEQ ID NO: 35 - protein, 28 aa, Artificial Sequence
  Description of Artificial Sequence  Synthetic peptide
CKTAVNCGSGCLTYYACGSGCVYNKAWC

SEQ ID NO: 36 - protein, 28 aa, Artificial Sequence
  Description of Artificial Sequence  Synthetic peptide
CVYNKAWCGSGCALITKCGSGCLTYYAC

SEQ ID NO: 37 - protein, 13 aa, Artificial Sequence
  Description of Artificial Sequence  Synthetic peptide
CLANFNCLTYYAC

SEQ ID NO: 38 - protein, 30 aa, Artificial Sequence
  Description of Artificial Sequence  Synthetic peptide
CFNDVTTRCGSGCLANFNCGSGCVYNKAWC

SEQ ID NO: 39 - protein, 7 aa, Artificial Sequence
  Description of Artificial Sequence  Synthetic peptide
CGLCGLC

SEQ ID NO: 40 - protein, 28 aa, Artificial Sequence
  Description of Artificial Sequence  Synthetic peptide
CLANFNCGSGCLTYYACGSGCVYNKAWC

SEQ ID NO: 41 - protein, 28 aa, Artificial Sequence
  Description of Artificial Sequence  Synthetic peptide
CLTYYACGSGCKTAVNCGSGCVYNKAWC

SEQ ID NO: 42 - protein, 13 aa, Artificial Sequence
  Description of Artificial Sequence  Synthetic peptide
CLTYYACLANFNC

SEQ ID NO: 43 - protein, 120 aa, Homo sapiens
MGIQGGSVLFGLLLVLAVFCHSGHSLQCYNCPNPTADCKTAVNCSSDFDACLITKAGLQVYNKCWKFEHCNFNDVTTRLRENELTYYCCKKDLCNFNEQLENGGTSLSEKTVLLLVTPFL

SEQ ID NO: 44 - DNA, 24 bp, Artificial Sequence
  Description of Artificial Sequence  Synthetic primer
atgragwcacakwcycaggtcttt

SEQ ID NO: 45 - DNA, 25 bp, Artificial Sequence
  Description of Artificial Sequence  Synthetic primer
atggagacagacacactcctgctat

SEQ ID NO: 46 - DNA, 29 bp, Artificial Sequence
  Description of Artificial Sequence  Synthetic primer
atggagwcagacacactsctgytatgggt

SEQ ID NO: 47 - DNA, 32 bp, Artificial Sequence
  Description of Artificial Sequence  Synthetic primer
  modified_base (27)..(27): Inosine
atgaggrcccctgctcagwttyttggnwtctt

SEQ ID NO: 48 - DNA, 31 bp, Artificial Sequence
  Description of Artificial Sequence  Synthetic primer
atgggcwtcaagatgragtcacakwyycwgg

SEQ ID NO: 49 - DNA, 29 bp, Artificial Sequence
  Description of Artificial Sequence  Synthetic primer
atgagtgtgcycactcaggtcctggsgtt

SEQ ID NO: 50 - DNA, 31 bp, Artificial Sequence
  Description of Artificial Sequence  Synthetic primer
atgtggggaycgktttyammcttttcaattg

SEQ ID NO: 51 - DNA, 28 bp, Artificial Sequence
  Description of Artificial Sequence  Synthetic primer
atggaagccccagctcagcttctcttcc

SEQ ID NO: 52 - DNA, 26 bp, Artificial Sequence
  Description of Artificial Sequence  Synthetic primer
  modified_base (6)..(6): Inosine
  modified_base (12)..(12): Inosine
  modified_base (18)..(18): Inosine
atgagnmmktcnmttcanttcytggg

SEQ ID NO: 53 - DNA, 26 bp, Artificial Sequence
  Description of Artificial Sequence  Synthetic primer
  modified_base (12)..(12): Inosine
  modified_base (24)..(24): Inosine
atgakgthcycngctcagytyctnrg

SEQ ID NO: 54 - DNA, 25 bp, Artificial Sequence
  Description of Artificial Sequence  Synthetic primer
atggtrtccwcasctcagttccttg

SEQ ID NO: 55 - DNA, 27 bp, Artificial Sequence
  Description of Artificial Sequence  Synthetic primer
atgtatatatgtttgttgtctatttct

SEQ ID NO: 56 - DNA, 29 bp, Artificial Sequence
  Description of Artificial Sequence  Synthetic primer
atgaagttgcctgttaggctgttggtgct

SEQ ID NO: 57 - DNA, 29 bp, Artificial Sequence
  Description of Artificial Sequence  Synthetic primer
atggatttwcargtgcagattwtcagctt

SEQ ID NO: 58 - DNA, 27 bp, Artificial Sequence
  Description of Artificial Sequence  Synthetic primer
atggtyctyatvtccttgctgttctgg

SEQ ID NO: 59 - DNA, 27 bp, Artificial Sequence
  Description of Artificial Sequence  Synthetic primer
atggtyctyatvttrctgctgctatgg

SEQ ID NO: 60 - DNA, 21 bp, Artificial Sequence
  Description of Artificial Sequence  Synthetic primer
actggatggtgggaagatgga

SEQ ID NO: 61 - DNA, 25 bp, Artificial Sequence
  Description of Artificial Sequence  Synthetic primer
atggcctggaytycwctywtmytct

SEQ ID NO: 62 - DNA, 23 bp, Artificial Sequence
  Description of Artificial Sequence  Synthetic primer
  modified_base (15)..(15): Inosine
agctcytcwgwgganggyggraa

SEQ ID NO: 63 - DNA, 25 bp, Artificial Sequence
  Description of Artificial Sequence  Synthetic primer
atgrasttskggytmarctkgrttt

SEQ ID NO: 64 - DNA, 26 bp, Artificial Sequence
  Description of Artificial Sequence  Synthetic primer
atgraatgsasctgggtywtyctctt

SEQ ID NO: 65 - DNA, 29 bp, Artificial Sequence
  Description of Artificial Sequence  Synthetic primer
atggactccaggctcaatttagttttcct

SEQ ID NO: 66 - DNA, 26 bp, Artificial Sequence
  Description of Artificial Sequence  Synthetic primer
atggctgtcytrgbgctgytcytctg

SEQ ID NO: 67 - DNA, 29 bp, Artificial Sequence
  Description of Artificial Sequence  Synthetic primer
atggvttggstgtggamcttgcyattcct

SEQ ID NO: 68 - DNA, 26 bp, Artificial Sequence
  Description of Artificial Sequence  Synthetic primer
atgaaatgcagctggrtyatsttctt

SEQ ID NO: 69 - DNA, 26 bp, Artificial Sequence
  Description of Artificial Sequence  Synthetic primer
atggrcagrcttacwtyytcattcct

SEQ ID NO: 70 - DNA, 26 bp, Artificial Sequence
  Description of Artificial Sequence  Synthetic primer
atgatggtgttaagtcttctgtacct

SEQ ID NO: 71 - DNA, 26 bp, Artificial Sequence
  Description of Artificial Sequence  Synthetic primer
atgggatggagctrtatcatsytctt

SEQ ID NO: 72 - DNA, 23 bp, Artificial Sequence
  Description of Artificial Sequence  Synthetic primer
atgaagwtgtggbtraactggrt

SEQ ID NO: 73 - DNA, 25 bp, Artificial Sequence
  Description of Artificial Sequence  Synthetic primer
  modified_base (15)..(15): Inosine
atggratggasckknrtctttmtct

SEQ ID NO: 74 - DNA, 25 bp, Artificial Sequence
  Description of Artificial Sequence  Synthetic primer
atgaacttygggytsagmttgrttt

SEQ ID NO: 75 - DNA, 25 bp, Artificial Sequence
  Description of Artificial Sequence  Synthetic primer
atgtacttgggactgagctgtgtat

SEQ ID NO: 76 - DNA, 23 bp, Artificial Sequence
  Description of Artificial Sequence  Synthetic primer
atgagagtgctgattcttttgtg

SEQ ID NO: 77 - DNA, 28 bp, Artificial Sequence
  Description of Artificial Sequence  Synthetic primer
atggattttgggctgattttttttattg

SEQ ID NO: 78 - DNA, 26 bp, Artificial Sequence
  Description of Artificial Sequence  Synthetic primer
  modified_base (21)..(21): Inosine
ccagggrccarkggataracngrtgg

SEQ ID NO: 79 - protein, 121 aa, Mus sp.
EVQLVESGGGLVKPGGSLKLSCAASGFAFSSYDMSWVRQTPKKRLEWVAYISSGGGSTHYPDTVKGRFTISRDNARNTLYLQMSSLKSDDTAMYYCARDGYYAEYYVMDYWGQGTSVTVSS

SEQ ID NO: 80 - protein, 107 aa, Mus sp.
DIQMTQSPASLSASVGETVTITCRASENIYSYLAWYQQKQGKSPQLLVYNAKTLAEGVPSRFSGSGSGTQFSLKINSLRPEDFGSYYCQHHYGSPLTFGAGTKLELR

SEQ ID NO: 81 - DNA, 39 bp, Artificial Sequence
  Description of Artificial Sequence  Synthetic oligonucleotide
gatcacgcgtgtccactccgaagtgcagctggtggagtc

SEQ ID NO: 82 - DNA, 33 bp, Artificial Sequence
  Description of Artificial Sequence  Synthetic oligonucleotide
gtacaagcttacctgaggagacggtgactgagg

SEQ ID NO: 83 - DNA, 53 bp, Artificial Sequence
  Description of Artificial Sequence  Synthetic oligonucleotide
gatcacgcgtgtccactccgaagtgcagctgctggagtctgggggaggcttag

SEQ ID NO: 84 - DNA, 52 bp, Artificial Sequence
  Description of Artificial Sequence  Synthetic oligonucleotide
ggagtctgggggaggcttagtgcagcctggagggtccctgagactctcctgt

SEQ ID NO: 85 - DNA, 30 bp, Artificial Sequence
  Description of Artificial Sequence  Synthetic oligonucleotide
ctccagcccctttcccggagcctggcgaac

SEQ ID NO: 86 - DNA, 30 bp, Artificial Sequence
  Description of Artificial Sequence  Synthetic oligonucleotide
gttcgccaggctccgggaaaggggctggag

SEQ ID NO: 87 - DNA, 53 bp, Artificial Sequence
  Description of Artificial Sequence  Synthetic oligonucleotide
cagccctcagactgttcatttgcaggtacagggtgtttttggaattgtctctg

SEQ ID NO: 88 - DNA, 52 bp, Artificial Sequence
  Description of Artificial Sequence  Synthetic oligonucleotide
caaatgaacagtctgagggctgaggacacagccgtgtattactgtgcacgcg

SEQ ID NO: 89 - DNA, 40 bp, Artificial Sequence
  Description of Artificial Sequence  Synthetic oligonucleotide
gatcaagcttacctgaggagacggtgactaaggttccttg

SEQ ID NO: 90 - DNA, 21 bp, Artificial Sequence
  Description of Artificial Sequence  Synthetic oligonucleotide
ctaatgtatgagacccactcc

SEQ ID NO: 91 - DNA, 21 bp, Artificial Sequence
  Description of Artificial Sequence  Synthetic oligonucleotide
ggagtgggtctcatacattag

SEQ ID NO: 92 - DNA, 35 bp, Artificial Sequence
  Description of Artificial Sequence  Synthetic oligonucleotide
catggcgcgcgatgtgacatccagatgactcagtc

SEQ ID NO: 93 - DNA, 63 bp, Artificial Sequence
  Description of Artificial Sequence  Synthetic oligonucleotide
tgcgggatccaactgaggaagcaaagtttaaattctactcacgtctcagctccagcttggtcc

SEQ ID NO: 94 - DNA, 50 bp, Artificial Sequence
  Description of Artificial Sequence  Synthetic oligonucleotide
catggcgcgcgatgtgacatccagatgactcagtctccatcctccctatc

SEQ ID NO: 95 - DNA, 48 bp, Artificial Sequence
  Description of Artificial Sequence  Synthetic oligonucleotide
cagtctccatcctccctatctgcatctgtgggagaccgtgtcaccatc

SEQ ID NO: 96 - DNA, 28 bp, Artificial Sequence
  Description of Artificial Sequence  Synthetic oligonucleotide
gaccaggagcttaggagcttttccctgt

SEQ ID NO: 97 - DNA, 28 bp, Artificial Sequence
  Description of Artificial Sequence  Synthetic oligonucleotide
acagggaaaagctcctaagctcctggtc

SEQ ID NO: 98 - DNA, 35 bp, Artificial Sequence
  Description of Artificial Sequence  Synthetic oligonucleotide
cttcaggctgcaggctgctgatcgtcagagtaaac

SEQ ID NO: 99 - DNA, 35 bp, Artificial Sequence
  Description of Artificial Sequence  Synthetic oligonucleotide
tcagcagcctgcagcctgaagattttgcgagttat

SEQ ID NO: 100 - DNA, 74 bp, Artificial Sequence
  Description of Artificial Sequence  Synthetic oligonucleotide
gatcggatccaactgaggaagcaaagtttaaattctactcacgtttgatctccagcttggtcccttgaccgaac

SEQ ID NO: 101 - DNA, 21 bp, Artificial Sequence
  Description of Artificial Sequence  Synthetic oligonucleotide
agcttttcccggtttctgctg

SEQ ID NO: 102 - DNA, 21 bp, Artificial Sequence
  Description of Artificial Sequence  Synthetic oligonucleotide
cagcagaaaccgggaaaagct

SEQ ID NO: 103 - DNA, 23 bp, Artificial Sequence
  Description of Artificial Sequence  Synthetic oligonucleotide
tcagagtaaagtctgtgcctgac

SEQ ID NO: 104 - DNA, 23 bp, Artificial Sequence
  Description of Artificial Sequence  Synthetic oligonucleotide
gtcaggcacagactttactctga

SEQ ID NO: 105 - DNA, 21 bp, Artificial Sequence
  Description of Artificial Sequence  Synthetic oligonucleotide
acagtaataagtcgcaaaatc

SEQ ID NO: 106 - DNA, 21 bp, Artificial Sequence
  Description of Artificial Sequence  Synthetic oligonucleotide
gattttgcgacttattactgt

SEQ ID NO: 107 - DNA, 21 bp, Artificial Sequence
  Description of Artificial Sequence  Synthetic oligonucleotide
gcattatagatcaggagctta

SEQ ID NO: 108 - DNA, 21 bp, Artificial Sequence
  Description of Artificial Sequence  Synthetic oligonucleotide
taagctcctgatctataatgc

SEQ ID NO: 109 - protein, 121 aa, Artificial Sequence
  Description of Artificial Sequence  Synthetic polypeptide
EVQLLESGGGLVQPGGSLRLSCAASGFAFSSYDMSWVRQAPGKGLEWVAYISSGGGSTHYPDTVKGRFTISRDNSKNTLYLQMNSLRAEDTAVYYCARDGYYAEYYVMDYWGQGTSVTVSS

SEQ ID NO: 110 - protein, 107 aa, Artificial Sequence
  Description of Artificial Sequence  Synthetic polypeptide
DIQMTQSPSSLSASVGDRVTITCRASENIYSYLAWYQQKPGKAPKLLVYNAKTLAEGVPSRFSGSGSGTDFTLTISSLQPEDFASYYCQHHYGSPLTFGQGTKLEIK

SEQ ID NO: 111 - protein, 107 aa, Artificial Sequence
  Description of Artificial Sequence  Synthetic polypeptide
DIQMTQSPSSLSASVGDRVTITCRASENIYSYLAWYQQKQGKAPKLLVYNAKTLAEGVPSRFSGSGSGTQFTLTISSLQPEDFASYYCQHHYGSPLTFGQGTKLEIK